(19) United States
(12) Patent Application Publication
(10) Pub. No.: US 2024/0376146 A1
(43) Pub. Date: Nov. 14, 2024

(54) PURIFICATION OF ANTIBODIES BY MIXED MODE CHROMATOGRAPHY

(71) Applicant: Hoffmann-La Roche Inc., Little Falls, NJ (US)

(72) Inventors: Roberto FALKENSTEIN, Muenchen (DE); Susanne KONRAD, Penzberg (DE)

(73) Assignee: Hoffmann-La Roche Inc., Little Falls, NJ (US)

(21) Appl. No.: 18/477,954

(22) Filed: Sep. 29, 2023

Related U.S. Application Data
(63) Continuation of application No. PCT/EP2022/058223, filed on Mar. 29, 2022.

(30) Foreign Application Priority Data
Mar. 31, 2021 (EP) 21166167.3

(51) Int. Cl. C07K 1/16

(52) U.S. Cl. CPC C07K 1/165

(57) ABSTRACT

Herein is reported a method for producing or purifying an antibody using a mixed mode chromatography material that comprises ion exchange functional groups and hydrophobic interaction functional groups (MM HIC/TEX) operated in flowthrough mode, wherein the antibody is a hydrophilic antibody, and the antibody is applied in a solution comprising the antibody and an antichaotropic salt to the MM HIC/IEX chromatography material.

### CROSS-REFERENCE TO RELATED APPLICATION

This application is a continuation of International Patent Application No. PCT/EP2022/058223, filed Mar. 29, 2022, which claims the benefit of EP patent application Ser. No. 21/166,167.3, filed Mar. 31, 2021, the contents of which are incorporated by reference herein in their entireties.

### FIELD OF THE INVENTION

The current invention is in the field of antibody purification. Especially, the current invention relates to methods for the production or purification of hydrophilic antibodies wherein the antibodies are processed in flow-through mode using a mixed mode (i.e. multimodal) chromatography material with ion exchange and hydrophobic interaction functionality. In particular, the methods involve the use of antichaotropic salts in the solution that is applied to the mixed mode chromatography material.

### BACKGROUND OF THE INVENTION

Monoclonal antibodies have proved to be a highly successful class of therapeutic products. For these recombinant biopharmaceutical proteins to be acceptable for administration to human patients, it is important that impurities resulting from the manufacture and purification process as well as impurities related to the product are removed from the final biological product. Process components include culture medium proteins, immunoglobulin affinity ligands, viruses, endotoxin, DNA, and host cell proteins (HCPs). Further impurities that are product related include low molecular weight (LMW) impurities like incompletely assembled antibodies or fragments. In addition also high molecular weight (HMW) impurities like dimers, trimers, multimers or in general aggregates can occur in production of pharmaceutical antibodies.

The phenomenon of protein aggregation is a common issue that compromises the quality, safety, and efficacy of antibodies and can happen at different steps of the manufacturing process. Aggregate levels in drug substance and final drug product are a key factor when assessing quality attributes of the molecule, since aggregation might impact biological activity of the biopharmaceutical. Differences in biological activity of the aggregates compared to the activity of the monomeric protein can significantly impair the potency of a protein-based drug.

During purification, chromatography is typically the step that mostly contributes to aggregate or HMW removals. The choice of a particular chromatography material and mode of operation should be guided by fit and compatibility with the overall process purification train as well as an appropriate balance of productivity, yield, and product quality. Protein A affinity chromatography is often used as the first purification step in manufacturing of therapeutic antibodies. This purification step is usually not or only scarcely capable of removing aggregates because product aggregates might bind to the chromatography ligand as well as monomer forms of the product. The use of ion (anion- and cation)-exchange chromatography has been demonstrated to be useful at production scale to separate antibody monomers from dimers and LMW species. WO 99/62936 reports the separation of monomers from aggregates by use of ion-exchange chromatography. It is possible to separate antibody monomers from aggregates based on differences in hydrophobicity by hydrophobic interaction chromatography (HIC), which has been mainly used for the removal of both aggregates and impurities such as HCP (Lu, Y. et al., 2009, Curr Pharm Biotechnol 10 (4): 427-433). Hydrophobicity of antibodies increases with aggregation, a fact that has significant theoretical as well as practical significance (Suda, E. J. et al., 2009, J Chromatogr A 1216 (27): 5256-5264). In addition, mixed mode or multimodal chromatography has been widely used for antibody purification and aggregate removal. For example Gagnon et al. (2009, Curr Pharm Biotechnol 10 (4): 434-439) report aggregate removal by charged-hydrophobic mixed mode chromatography. Also Gao et al. (2013, Journal of Chromatography A, 1294 70-75) describe antibody monomer separation from associated aggregates using mixed-mode chromatography.

In addition to the reduction of product-related impurities like HMWs or aggregates, also process related impurities like HCPs or virus particles need to be removed during purification. Viral contamination is a potential risk of using biotechnology products derived from mammalian cell lines. Therefore, to provide assurance of the safety of these products regarding potential viral contamination, regulatory authorities require viral clearance studies assessing the ability of the purification process to clear endogenous and exogenous viruses. For the removal of viral contamination often virus filters and low pH inactivation are used but also chromatography processes like anion exchange chromatography may be useful (Ajayi et al., 2022, Current Research in Biotechnology 4:190-202).

Despite these advances in purification in general and in particular of antibody monomers from HMW impurities as well as removal of viruses by means of different chromatography materials there is still the need and room for improving the purification settings to achieve even higher purity and quality of antibodies.

### SUMMARY OF THE INVENTION

Herein is reported a method for the purification or production of a hydrophilic antibody with a mixed mode/multimodal chromatography material that comprises ion exchange functional groups and hydrophobic interaction functional groups in the presence of an antichaotropic salt.

The present invention is based, at least in part, on the unexpected finding that antibody related high molecular weight impurities (HMWs) can be successfully reduced when the load solution comprising a hydrophilic antibody and HMWs is purified by a mixed mode chromatography material that comprises ion exchange functional groups and hydrophobic interaction functional groups (MM HIC/IEX) in flowthrough (FT) mode in the presence of at least one antichaotropic salt. In one preferred embodiment of the invention, the antichaotropic salt is present in the equilibration (buffer) used to equilibrate the chromatography material, the load solution and optional washing/rising solutions. It has been found that the production and/or purification, i.e. the reduction of HMWs, can be performed for hydrophilic antibodies in the presence of an antichaotropic salt but that the effect cannot be achieved for hydrophobic antibodies or in the presence of a chaotropic salt.

Further, the present invention is based, at least in part, on the unexpected finding that also contaminations with viruses or virus-like particles (e.g. RVLPs) i.e. the viral impurity content can be successfully reduced when the load solution comprising a hydrophilic antibody and a viral impurity is purified by a mixed mode chromatography material that comprises ion exchange functional groups and hydrophobic interaction functional groups (MM HIC/IEX) in flowthrough (FT) mode in the presence of at least one antichaotropic salt. In one preferred embodiment of the invention, the antichaotropic salt is present in the equilibration (buffer) used to equilibrate the chromatography material, the load solution and optional washing/rising solutions. It has been found that the production and/or purification, i.e. the reduction of viral impurity content, can be performed for hydrophilic antibodies in the presence of an antichaotropic salt but that the effect is not present for hydrophobic antibodies.

Thus, one aspect of the invention is a method for producing an antibody using (/with) a mixed mode/multimodal chromatography material that comprises ion exchange functional groups and hydrophobic interaction functional groups (MM HIC/IEX) operated in flowthrough mode, wherein
a) the antibody is a hydrophilic antibody, and
b) the antibody is applied in a solution comprising the antibody and an antichaotropic salt to the MM HIC/IEX.

In certain embodiments of the above aspect and the other embodiments the method further comprises the following steps:
c) optionally a rinsing solution is applied,
d) the antibody is recovered in the flowthrough of b) or optionally in the flowthrough of b) and c),
   and thereby producing the antibody using a MM HIC/IEX operated in flowthrough mode.

Another aspect according to the invention is a method for purifying an antibody using (with) a mixed mode/multimodal chromatography material that comprises ion exchange functional groups and hydrophobic interaction functional groups (MM HIC/IEX) operated in flowthrough mode, wherein
a) the antibody is a hydrophilic antibody, and
b) the antibody is applied in a solution comprising the antibody and an antichaotropic salt to the MM HIC/IEX,
   and thereby purifying the antibody.

In certain embodiments of the above aspects and the other embodiments the MM HIC/IEX has been conditioned/equilibrated with a buffer comprising the (same) antichaotropic salt. In one preferred embodiment, the buffer used for conditioning/equilibrating the MM HIC/IEX is also the buffer of the solution of step b).

In certain embodiments of the above aspects and the other embodiments
the method is for producing an antibody composition with reduced antibody-related high molecular weight (HMW) impurity content and/or with reduced viral impurity content,
the antibody is applied to the MM HIC/IEX in a solution comprising the antibody, at least one antibody-related HMW impurity and/or at least one viral impurity and an antichaotropic salt,
the antibody composition with reduced antibody-related HMW impurity content and/or with reduced viral impurity content is recovered from the flowthrough, and
thereby an antibody composition with reduced antibody-related HMW impurity content and/or with reduced viral impurity content is produced.

In certain embodiments of the above aspects and the other embodiments
the method is for producing an antibody composition with reduced antibody-related high molecular weight (HMW) impurity content and with reduced viral impurity content,
the antibody is applied to the MM HIC/IEX in a solution comprising the antibody, at least one antibody-related HMW impurity and at least one viral impurity and an antichaotropic salt,
the antibody composition with reduced antibody-related HMW impurity content and with reduced viral impurity content is recovered from the flowthrough, and
thereby an antibody composition with reduced antibody-related HMW impurity content and with reduced viral impurity content is produced.

In certain embodiments of the above aspects and the other embodiments
the method is for producing an antibody composition with reduced antibody-related high molecular weight (HMW) impurity content,
the antibody is applied to the MM HIC/IEX in a solution comprising the antibody, at least one antibody-related HMW impurity and an antichaotropic salt,
the antibody composition with reduced antibody-related HMW impurity content is recovered from the flowthrough, and
thereby an antibody composition with reduced antibody-related HMW impurity content is produced.

In certain embodiments of the above aspects and the other embodiments the antibody-related (HMW) impurity content is reduced compared to the solution applied to the MM HIC/IEX in step b).

In certain embodiments of the above aspects and the other embodiments the antibody-related (HMW) impurity content is reduced compared to a solution essentially without an antichaotropic salt; and/or compared to a solution comprising a hydrophobic antibody.

In certain embodiments of the above aspects and the other embodiments
the method is for producing an antibody composition with reduced viral impurity content,
the antibody is applied to the MM HIC/IEX in a solution comprising the antibody, at least one viral impurity and an antichaotropic salt,
the antibody composition with reduced viral impurity content is recovered from the flowthrough, and
thereby an antibody composition with reduced viral impurity content is produced.

In certain embodiments of the above aspects and the other embodiments the viral impurity content is reduced compared to the solution applied to the MM HIC/IEX in step b).

In certain embodiments of the above aspects and the other embodiments the viral impurity content is reduced compared to a solution essentially without an antichaotropic salt; and/or compared to a solution comprising a hydrophobic antibody.

In certain embodiments of the above aspects and the other embodiments the hydrophilic antibody is an antibody that has a retention time on a hydrophobic interaction chromatography (HIC) material that is equal or less than that of rituximab (on the same HIC material and under the same operating conditions).

In certain embodiments of the above aspects and the other embodiments the hydrophobic interaction chromatography (HIC) material contains polyether groups (ethyl ether groups) as ligand.

In certain embodiments of the above aspects and the other embodiments the hydrophobic interaction chromatography (HIC) material contains polyether groups with the following structure (—(OCH₂CH₂)ₙOH) as ligand.

In certain embodiments of the above aspects and the other embodiments the hydrophobic interaction chromatography (HIC) material contains a polymethacrylate base material/matrix.

In certain embodiments of the above aspects and the other embodiments the hydrophobic interaction chromatography (HIC) material contains polyether groups (ethyl ether groups) as ligand, has a mean pore size of 100 nm and a particle size of 10 μm.

In certain embodiments of the above aspects and the other embodiments the hydrophobic interaction chromatography (HIC) material is a TSKgel® Ether-5 PW chromatography material.

In certain embodiments of the above aspects and the other embodiments the retention time on the HIC chromatography material is determined using the HIC chromatography material (of any one of the other embodiments), and with a column length of 75 mm, and with an inner diameter of 7.5 mm, and with an elution buffer gradient at a flow rate of 8.8 ml/min, and wherein the antibody is applied to the chromatography material at a concentration of 1 mg/ml.

In certain embodiments of the above aspects and the other embodiments the antichaotropic salt has a molar surface tension increment in the range of and including 1.285 to 4.183×10E3 dyn*g*cm⁻¹*mol⁻¹

In certain embodiments of the above aspects and the other embodiments the antichaotropic salt is selected from the group consisting of chlorides, sulfates, citrates, carbonates, phosphates, acetates or fluorides.

In certain embodiments of the above aspects and the other embodiments the antichaotropic salt is a calcium-, sodium-, ammonium- or potassium-salt.

In certain embodiments of the above aspects and the other embodiments the antichaotropic salt is a sodium-, ammonium- or potassium-salt.

In certain embodiments of the above aspects and the other embodiments the antichaotropic salt is selected from the group consisting of (NH₄)₂SO₄, Na₂SO₄, K₂SO₄, NaCl and KCl.

In certain embodiments of the above aspects and the other embodiments the solution comprising the antibody and an antichaotropic salt (of step b) has a conductivity of from (and including) 0.5 to 120 mS/cm.

In certain embodiments of the above aspects and the other embodiments in the solution comprising the antibody and an antichaotropic salt, the antichaotropic salt has a concentration of from (and including) 10 mM to 900 mM.

In certain embodiments of the above aspects and the other embodiments the loaded amount to the MM HIC/IEX is 10 g of protein per Liter of chromatography material (10 g/L) or higher.

In certain embodiments of the above aspects and the other embodiments the loaded amount to the MM HIC/IEX is from (and including) 10 g of protein per Liter of chromatography material (10 g/L) to 650 g of protein per Liter of chromatography material (650 g/L).

In certain embodiments of the above aspects and the other embodiments the loaded amount to the MM HIC/IEX is from (and including) 15 g of protein per Liter of chromatography material (15 g/L) to 350 g of protein per Liter of chromatography material (350 g/L).

In certain embodiments of the above aspects and the other embodiments the solution comprising the antibody and an antichaotropic salt has a pH value of from (and including) 4.0 to 9.0.

In certain embodiments of the above aspects and the other embodiments the HMW impurity is an impurity which has a molecular weight of 285 kDa or more.

In certain embodiments of the above aspects and the other embodiments the HMW impurity is an impurity which is at least a dimer, or a trimer, or any multimer of the antibody.

In certain embodiments of the above aspects and the other embodiments the MM HIC/IEX comprises anion exchange functional groups or cation exchange functional groups.

In certain embodiments of the above aspects and the other embodiments the MM HIC/IEX comprises strong anion exchange functional groups.

In certain embodiments of the above aspects and the other embodiments the MM HIC/IEX is Capto™ adhere ImpRes, Capto™ Adhere or Nuvia aPrime4A.

In certain embodiments of the above aspects and the other embodiments the MM HIC/IEX comprises weak cation exchange functional groups.

In certain embodiments of the above aspects and the other embodiments the MM HIC/IEX is Capto™ MMC or Capto™ MMC ImpRes.

### DETAILED DESCRIPTION OF THE INVENTION

Herein is reported a method for the purification or production of a hydrophilic antibody using a mixed mode/multimodal chromatography material that comprises ion exchange functional groups and hydrophobic interaction functional groups and with the use of an antichaotropic salt in the load solution (denoted also as “load” herein).

The present invention is based, at least in part, on the unexpected finding that antibody related high molecular weight impurities (HMWs) can be successfully reduced when the load comprising a hydrophilic antibody and HMWs is purified by a mixed mode chromatography material that comprises ion exchange functional groups and hydrophobic interaction functional groups (MM HIC/IEX) in flowthrough (FT) mode and there is at least one antichaotropic salt comprised in the load solution prior to the start of the chromatography.

In more detail and surprisingly, it has been found that the content of HMWs can be reduced significantly more, when the to-be-purified antibody is a hydrophilic antibody and an antichaotropic salt is present. In contrast, when hydrophobic antibodies are being purified, no significant effect of the addition of an antichaotropic salt regarding HMW reduction can be observed.

The invention is further based, at least in part, on the finding that the effect is not attributable to an increase in salt molarity per se. Again, a positive effect for hydrophilic antibodies could be observed with increasing salt molarities of antichaotropic salts, while this effect could not be observed for hydrophobic antibodies.

The invention is further based, at least in part, on the finding that an improved HMW reduction could not be shown for chaotropic salts.

In the first set of experiments (part I) the impact of antibody hydrophobicity on HMW impurity reduction at constant conductivity was shown.

Flowthrough (FT) runs were performed with Capto™ adhere ImpRes Robocolumns™ (RCs) on a robotic system. Five antichaotropic (ac) salts were used: Na₂SO₄, NaCl, (NH₄)₂SO₄, KCl, and K₂SO₄. These were used in combination with seven monoclonal antibodies (mabs) of different format and specificity. The FT was collected and purity was analyzed by SE-HPLC. The HMW reduction achieved by adding an antichaotropic salt to the load were compared with the HMW reduction achieved with the same buffer, i.e. at same conductivity, but without containing an antichaotropic salt. It has been found that only for hydrophilic mabs, i.e. mabs with a retention time determined in a HIC chromatography according to Material and Methods item 10 (MM-10) of less than that of Rituximab (retention time_{mab}≤retention time_{rituximab}), an improved HMW removal was achieved by addition of an antichaotropic salt to the load compared to a load having the same conductivity in the absence of an antichaotropic salt. In contrast to that, it has been found that for hydrophobic mabs (retention time_{mab}>retention time_{rituximab}) no advantageous effect was observed with the addition of an antichaotropic salt.

For multiple hydrophilic antibodies it has been found that HMW reduction was improved when an antichaotropic salt was added to the load solution compared to a load solution in Tris/Acetate buffer without an antichaotropic salt at same conductivity (see FIGS. 1 to 4; black filled circles). It has been found that the presence of an antichaotropic salt in the load resulted in an improved HMW reduction for hydrophilic mabs on a MM HIC/IEX.

In contrast to that, for hydrophobic antibodies HMW reduction was not improved for loads containing an antichaotropic salt and for loads without an antichaotropic salt (see FIGS. 5 to 7).

To calculate the HMW removal for pools, trend lines were introduced. The HMW removal for the FT pools is shown for an exemplary hydrophilic mab (FIG. 8A; mab2) and for an exemplary hydrophobic mab (FIG. 8B; mab7). For mab2 the pool HMW removal value at a total load of 150 g/L increased from 35% to 89% when ammonium sulfate was added to the load (see FIG. 9A). For a total load of 550 g/L the pool HMW removal value increased from 17% to 47% with the addition of (NH₄)₂SO₄ to the load. In contrast to that, the pool HMW values for mab7 with and without an antichaotropic salt were similar. For this hydrophobic mab the pool HMW removal value was not significantly improved by addition of an antichaotropic salt (see FIG. 9B).

The same effect can be seen at different pH values (Example 1: pH 8; Example 2: pH 6). It has been found that for hydrophilic mabs HMW reduction was significantly improved when an antichaotropic salt was added to the load solution (see FIGS. 10 to 13). In contrast to that, for hydrophobic mabs HMW reduction for loads containing an antichaotropic salt and for loads without an antichaotropic salt was comparable (see FIGS. 14 to 16).

The same effect can be seen at different conductivities (Example 1/2: 20 mS/cm); Example 3:10 mS/cm) (see FIGS. 17(A and B) and 18(A and B). It has been found that the presence of an antichaotropic salt for a hydrophilic mab improved the HMW reduction in the FT fractions. For a hydrophobic mab, no improvement in HMW reduction was observed when an antichaotropic salt was added to the load. It has been found that the effect of an improved HMW reduction for hydrophilic mabs by adding an antichaotropic salt to the load was more pronounced with increasing pH.

In the second set of experiments the impact of antichaotropic salt molarity on HMW removal has been shown.

RC experiments and Kp (partition coefficient) screens were performed to show the effect of different molarities of antichaotropic salts on HMW reduction. A molarity of up to 500 mM of salt has been used. Different salts, Na₂SO₄, NaCl, (NH₄)₂SO₄, KCl and K₂SO₄ were tested at pH 8.0. It has been found that an increase in salt molarity could improve removal of HMWs in the FT fractions of hydrophilic antibody preparations (see FIGS. 19 to 23).

Further, Kp screens showed the effect of salt molarity on HMW reduction for a broad range of pH values and salt molarities (see Examples 5 and 6 using Capto™ adhere ImpRes resin/chromatography material). Three mabs were used, two hydrophilic mabs and one hydrophobic mab. The investigated pH range was pH 5.5-8.0 and the molarity range was 10-800 mM. The Kp screens confirmed the RC data with respect to HMW reduction. It has been found that for hydrophilic mabs an increasing salt molarity resulted in an improved HMW reduction whereas HMW reduction for the hydrophobic mab was not improved by increasing the antichaotropic salt molarity (see FIGS. 24A to 25C). As expected, using a chaotropic salts did not show an improved HMW reduction with increasing salt molarity (see FIGS. 26A to 27C).

Further, Kp screens were performed with a pH range of pH 4 to 9 and salt molarities up to ˜900 mM (see Example 6). Within these Kp screens two sets of buffers were compared: one buffer containing the antichaotropic salt Na₂SO₄ and one buffer without an antichaotropic salt (see FIGS. 28A and 28B).

In more detail, the effect of increasing salt molarity (and conductivity) was tested for five antichaotropic salts at pH 8 with a hydrophilic mab (Example 4; mab2). The antichaotropic salts were sodium sulfate (see FIG. 19 for results), sodium chloride (see FIG. 20 for results), ammonium sulfate (see FIG. 21 for results), potassium chloride (see FIG. 22 for results) and potassium sulfate (see FIG. 23 for results). The achieved HMW removal values for each FT fraction were plotted against the increasing total loaded amount. In general, an increase in HMW removal values with increasing total loaded amount was observed. It has been found that by adding an antichaotropic salt to a hydrophilic mab a decrease in HMW level of the FT fractions could be achieved. An improved HMW reduction was found for all tested antichaotropic salts.

Moreover, the effect of increasing salt molarity was verified for three mabs (hydrophilic and hydrophobic) and four salts, (NH₄)₂SO₄, KCl, Gua/HCl and Urea, using Kp screens in the pH range of 5.5 to 8.0 and a salt molarity up to 800 mM (see Example 5). The chaotropic salts (Gua/HCl and urea) were chosen to show the HMW reduction when hydrophobic interactions are weakened.

Depending on the hydrophobicity of the mabs, differences in the HMW removals were seen. It has been found that for the hydrophilic mabs (mab2; A and mab4; B) the addition of an antichaotropic salt (ammonium sulfate, FIGS. 24A and 24B, and KCl, FIGS. 25A and 25B) HMW reduction of up to 70 to 80% was achieved. For the hydrophobic mab (mab6; C) HMW reduction in the presence of ammonium sulfate was nearly unaffected by molarity. With KCl, the HMW reduction for hydrophobic mab6 even decreased with increasing KCl molarity. FIGS. 24C and 25C show that for hydrophobic mab6 HMW removal was not improved by increasing molarity of an antichaotropic salt.

Gua/HCl (see FIGS. 26A, 26B and 26C) and urea (see FIGS. 27A, 27B and 27C) were used to determine the effect of a chaotropic salt on HMW reduction. No improvement of HMW reduction was observed with increasing salt molarity for hydrophilic as well as hydrophobic mabs.

In summary it has been found that HMW reduction was improved for hydrophilic mabs when an antichaotropic salt was added to the load solution. For hydrophobic mabs, no improved HMW reduction could be observed by addition of an antichaotropic salt. Furthermore, an improved HMW reduction could not be obtained with addition of chaotropic salts.

The results of Example 6 show that the addition of an antichaotropic salt improved the HMW removal in FT fractions for a hydrophilic mab (mab2). An increasing Na₂SO₄ molarity (see FIG. 28A) showed an improved HMW reduction up to 80%. The contour plot of mab2 with Na₂SO₄ was similar to that with ammonium sulfate (see FIG. 24A). In contrast to that an increase in Tris/Acetate molarity (see FIG. 28B) had no significant impact on HMW reduction. Without addition of an antichaotropic salt, no improved HMW reduction was observed with increasing molarity.

In the third set of experiments (part III) different chromatography resins were used.

In example 7 HMW reduction in loads comprising a hydrophilic mab (mab2) in a pH range of 5.5-8.0 and salt molarities of 10-800 mM was investigated. Three different mixed mode anion exchange (MMAEX) resins were used: Capto™ adhere ImpRes, Capto™ adhere and Nuvia aPrime. It has been found that all three chromatography materials showed an improved HMW reduction when an antichaotropic salt was added to the load solution comprising a hydrophilic mab.

Example 8 summarizes Kp screens done for a MMAEX resin, an anion exchange (AEX) resin, a HIC resin and a mixed mode cation exchange (MMCEX) resin with a hydrophilic and a hydrophobic mab. The used pH range was pH 4.0-9.0 and the used salt concentration was 5-850 mM. It has been found that the flowthrough samples of the hydrophilic mab show improved HMW reduction with increasing salt molarity for both ionic mixed mode resins (MMAEX and MMCEX). In contrast to that for the hydrophobic mab HMW reduction on the MMAEX resin was independent of salt molarity. For the MMCEX resin HMW reduction for the hydrophobic mab was not improved by increasing salt molarity below 500 mM Na₂SO₄. With the single mode resins Q Sepharose FF (AEX) no advantageous effect on HMW reduction was observed neither for the hydrophilic nor for the hydrophobic mab. For the Phenyl Sepharose 6 FF (high sub) (HIC) a positive effect of increasing salt molarity on HMW reduction has been found for the hydrophilic mab but (in contrast to the mixed mode resins) also for the hydrophobic mab.

In more detail, mab2 was used in Example 7 with different mixed mode resins. Three mixed mode resins with anion exchange and hydrophobic interaction were used. Capto™ adhere ImpRes flowthrough contour plots are shown in FIGS. 29A, 30A, 31A and 32A, the contour plots of Capto™ adhere are shown in FIGS. 29B, 30B, 31B and 32B and those of Nuvia aPrime are shown in FIGS. 29C, 30C, 31C and 32C. Two antichaotropic salts, (NH₄)₂SO₄ (see FIGS. 29A, 29B and 29C) and KCl (see FIGS. 30A, 30B and 30C), and two chaotropic salts, Gua/HCl (see FIGS. 31A, 31B and 31C) and Urea (see FIGS. 32A, 32B and 32C), were tested.

In general, it has been found that for all salts the contour plots of Capto™ adhere, Nuvia aPrime as well as Capto™ adhere ImpRes showed comparable results. With increasing (NH₄)₂SO₄ and KCl molarity, all three mixed mode resins showed an improved HMW reduction. All contour plots showed a good comparability. For the chaotropic salts no improved HMW reduction was observed with addition of the respective salt.

In summary, the improved HMW reduction can be achieved with different MMAEX resins.

In Example 8, one hydrophilic mab (mab2) and one hydrophobic mab (mab6) were used in combination with different resin types: a mixed mode anion exchange resin (Capto™ adhere ImpRes), an anion exchange resin (Q Sepharose FF), a hydrophobic resin (Phenyl Sepharose 6 FF) and a mixed mode cation exchange resin (Capto™ MMC ImpRes).

For the AEX resin Q Sepharose FF no effect of an antichaotropic salt on HMW reduction was observed (see FIGS. 34A and 34B). For the resin Phenyl Sepharose 6FF (high sub) both the hydrophilic but also the hydrophobic mab showed an improved HMW reduction with increasing Na₂SO₄ molarity (see FIGS. 35A and 35B). Thus, for the single mode resins Q Sepharose FF and Phenyl Sepharose 6FF (high sub) the HMW reduction achievable for the hydrophilic and hydrophobic mabs were comparable.

In contrast to that, for the mixed mode resins HMW reduction for the hydrophilic and hydrophobic mab were different. The MMAEX resin showed an improved HMW removal for the hydrophilic mab2 (see FIG. 33A) when an antichaotropic salt was added to the load. For the hydrophobic mab6 the HMW removal was almost constant over the tested pH and molarity range (see FIG. 33B). Thus, it has been found that only for the hydrophilic mab HMW reduction has been enhanced by increasing salt molarity on a mixed mode anion exchange resin. FIGS. 36A and 36B illustrates the HMW removal values for the Capto™ MMC ImpRes resin. For the hydrophilic mab HMW reduction was enhanced with increasing Na₂SO₄ molarity in the range of 0 to 800 mM from 20% up to 80%. In contrast to that, HMW reduction for the hydrophobic mab was unaffected by increasing salt molarity up to 500 mM. For mab6 an improved HMW reduction in the FT samples was observed only for molarities higher than 500 mM. Below 500 mM HMW reduction was poor (<10%) and independent of salt molarity.

Without being bound by this theory, Example 8 shows that an improved HMW reduction for a hydrophilic mab by addition of an antichaotropic salt could be attributed to the combination of ionic and hydrophobic interactions (but not for hydrophobic interactions alone). For both ionic mixed mode resins, the Capto™ adhere ImpRes (with anionic and hydrophobic moieties) and the Capto™ MMC ImpRes (with cationic and hydrophobic moieties), an improved HMW reduction has been achieved with increasing salt molarity but only for hydrophilic mabs.

The method is also freely scalable (part IV; see Examples 9 and 10).

The scale-up results confirmed the results achieved with the smaller volume systems in parts II and III.

In more detail, in Example 9 two loads of mab2 were prepared with the same conductivity of 9 mS/cm, but different molarities of Na₂SO₄ (40 mM and 20 mM). The FT fractions of the load with the higher Na₂SO₄ molarity resulted in higher mainpeak values compared to the load with lower Na₂SO₄ molarity (see FIG. 37). This shows that the higher Na₂SO₄ molarity (and not the conductivity) enhanced HMW removal as the load conductivity was the same.

Example 10 showed the effect of Na₂SO₄ molarity on HMW reduction for pH 7 and pH 8. The mainpeak values of the FT fractions increased with increasing Na₂SO₄ molarity at pH 7 (see FIG. 39) as well as at pH 8 (see FIG. 38). FT pools were calculated using the average mainpeak value of the fractions at pH 8 (see FIG. 40). For pH 8 the mainpeak value increased from 96.96% (without Na₂SO₄ conductivity of 5 mS/cm) up to 99.09% with a load containing 60 mM Na₂SO₄ (conductivity of 12 mS/cm).

In the fifth set of experiments the impact of antichaotropic salt molarity on virus removal/RVLP removal has been shown.

Kp (partition coefficient) screens were performed to show the effect of different molarities of antichaotropic salts on viral contaminant reduction. The effect of mab hydrophobicity and the presence of an antichaotropic salt was shown with two hydrophilic and two hydrophobic mabs in the pH range of pH 5.0-8.0. In Example 11 the antichaotropic salt sodium sulfate with a salt molarity up to 400 mM was investigated. In Example 12 a Tris/Acetate buffer with increasing Tris molarity and increasing conductivity, but lacking an antichaotropic salt, was used.

Depending on the hydrophobicity of the mab and the presence of an antichaotropic salt, different RNA reduction values (representive of RVLP reduction which in turn is a surrogate measurement for viral contaminant reduction) were measured.

In summary it has been found that viral contaminant reduction was improved for hydrophilic mabs when an antichaotropic salt was added to the load solution. For hydrophobic mabs, no improved viral contaminant reduction could be observed by addition of an antichaotropic salt.

Thus, the present invention is based, at least in part, on the unexpected finding that antibody related high molecular weight impurities (HMWs) can be successfully reduced when the load solution comprising a hydrophilic antibody and HMWs is purified by a mixed mode chromatography material that comprises ion exchange functional groups and hydrophobic interaction functional groups (MM HIC/IEX) in flowthrough (FT) mode and there is at least one antichaotropic salt comprised in the load solution prior to the start of the chromatography.

Furthermore, the present invention is based, at least in part, on the unexpected finding that viral contaminants can be successfully reduced when the load comprising a hydrophilic antibody and viral contaminants is purified by a mixed mode chromatography material that comprises ion exchange functional groups and hydrophobic interaction functional groups (MM HIC/IEX) in flowthrough (FT) mode and there is at least one antichaotropic salt comprised in the load solution prior to the start of the chromatography.

In more detail and surprisingly, it has been found that the content of viral contaminants can be reduced significantly more, when the to-be-purified antibody is a hydrophilic antibody and an antichaotropic salt is present. In contrast, when hydrophobic antibodies are being purified, no significant effect of the addition of an antichaotropic salt regarding viral contaminant reduction can be observed.

The invention is further based, at least in part, on the finding that the effect is not attributable to an increase in salt molarity per se. Again, a positive effect for hydrophilic antibodies could be observed with certain salt molarities of antichaotropic salts, while this effect could not be observed for hydrophobic antibodies.

Therefore, one aspect according to the invention is a method for producing an antibody using (/with) a mixed mode/multimodal chromatography material that comprises ion exchange functional groups and hydrophobic interaction functional groups (MM HIC/IEX) operated in flowthrough mode, wherein
a) the antibody is a hydrophilic antibody, and
b) the antibody is applied in a solution comprising the antibody and an antichaotropic salt to the MM HIC/IEX chromatography material.

In certain embodiments of the above aspect and the other embodiments the method further comprises the following steps:
c) optionally a rinsing solution is applied,
d) the antibody is recovered in the flowthrough of b) or optionally in the flowthrough of b) and c), and thereby producing the antibody using a MM HIC/IEX operated in flowthrough mode.

Another aspect according to the invention is a method for purifying an antibody using (with) a mixed mode/multimodal chromatography material that comprises ion exchange functional groups and hydrophobic interaction functional groups (MM HIC/IEX) operated in flowthrough mode, wherein
a) the antibody is a hydrophilic antibody, and
b) the antibody is applied in a solution comprising the antibody and an antichaotropic salt to the MM HIC/IEX chromatography material, and thereby purifying the antibody.

### Definitions and Embodiments

The term “antibody” herein is used in the broadest sense and specifically covers monoclonal antibodies, polyclonal antibodies, multispecific antibodies (e.g. bispecific antibodies) formed from at least two intact antibodies. Antibody fragments as well as fusion polypeptides, as long as they do possess an Fc-region are encompassed by this definition. The term “immunoglobulin” (Ig) is used interchangeable with antibody herein.

Antibodies are naturally occurring immunoglobulin molecules which have varying structures, all based upon the immunoglobulin fold. For example, IgG antibodies have two “heavy” chains and two “light” chains that are disulfide-bonded to form a functional antibody. Each heavy and light chain itself comprises a “constant” (C) and a “variable” (V) region. The V regions determine the antigen binding specificity of the antibody, whilst the C regions provide structural support and function in non-antigen-specific interactions with immune effectors. The antigen binding specificity of an antibody or antigen-binding fragment of an antibody is the ability of an antibody to specifically bind to a particular antigen.

The antigen binding specificity of an antibody is determined by the structural characteristics of the V region. The variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

Each V region typically comprises three complementarity determining regions (“CDRs”, each of which contains a “hypervariable loop”), and four framework regions. An antibody binding site, the minimal structural unit required to bind with substantial affinity to a particular desired antigen, will therefore typically include the three CDRs, and at least three, preferably four, framework regions interspersed there between to hold and present the CDRs in the appropriate conformation. Classical four chain antibodies have antigen binding sites which are defined by VH and VL domains in cooperation. Certain antibodies, such as camel and shark antibodies, lack light chains and rely on binding sites formed by heavy chains only.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, MD. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody that are responsible for antigen binding. The hypervariable region may comprise amino acid residues from a “complementarity determining region” or “CDR” (e.g., around about residues 24-34 (L1), 50-56 (L2) and 89-97 (L3) in the VL, and around about 31-35B (H1), 50-65 (H2) and 95-102 (H3) in the VH (Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the VL, and 26-32 (H1), 52A-55 (H2) and 96-101 (H3) in the VH (Chothia and Lesk J. Mol. Biol. 196:901-917 (1987)).

“Framework” or “FR” residues are those variable domain residues other than the hypervariable region residues as herein defined.

“Hinge region” in the context of an antibody or half-antibody is generally defined as stretching from Glu216 to Pro230 of human IgG1 (Burton, Molec. Immunol. 22:161-206 (1985)). Hinge regions of other IgG isotypes may be aligned with the IgG1 sequence by placing the first and last cysteine residues forming inter-heavy chain S—S bonds in the same positions.

The “lower hinge region” of an Fc region is normally defined as the stretch of residues immediately C-terminal to the hinge region, i.e. residues 233 to 239 of the Fc region. Prior to the present application, FcγR binding was generally attributed to amino acid residues in the lower hinge region of an IgG Fc region.

The “CH2 domain” of a human IgG Fc region usually extends from about residues 231 to about 340 of the IgG. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec. Immunol. 22:161-206 (1985).

The “CH3 domain” comprises the stretch of residues C-terminal to a CH2 domain in an Fc region (i.e. from about amino acid residue 341 to about amino acid residue 447 of an IgG).

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH1). Pepsin treatment of an antibody yields a single large F(ab′)2 fragment which roughly corresponds to two disulfide linked Fab fragments having divalent antigen-binding activity and is still capable of cross-linking antigen. Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

“Fv” is the minimum antibody fragment that contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)2 antibody fragments originally were produced as pairs of Fab′ fragments that have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy chain constant domains that correspond to the different classes of antibodies are called α, δ, ε, γ, and μ, respectively. The subunit structures and three-dimensional configurations of different classes of immunoglobulins are well known.

The term “half-antibody” as used herein refers to a monovalent antigen binding polypeptide. In certain embodiments, a half antibody comprises a VH/VL unit and optionally at least a portion of an immunoglobulin constant domain. In certain embodiments, a half antibody comprises one immunoglobulin heavy chain associated with one immunoglobulin light chain, or an antigen binding fragment thereof. In certain embodiments, a half antibody is mono-specific, i.e., binds to a single antigen or epitope. One skilled in the art will readily appreciate that a half-antibody may have an antigen binding domain consisting of a single variable domain, e.g., originating from a camelidae.

The term “VH/VL unit” refers to the antigen-binding region of an antibody that comprises at least one VH HVR and at least one VL HVR. In certain embodiments, the VH/VL unit comprises at least one, at least two, or all three VH HVRs and at least one, at least two, or all three VL HVRs. In certain embodiments, the VH/VL unit further comprises at least a portion of a framework region (FR). In some embodiments, a VH/VL unit comprises three VH HVRs and three VL HVRs. In some such embodiments, a VH/VL unit comprises at least one, at least two, at least three or all four VH FRs and at least one, at least two, at least three or all four VL FRs.

The term “multispecific antibody” is used in the broadest sense and specifically covers an antibody comprising an antigen-binding domain that has polyepitopic specificity (i.e., is capable of specifically binding to two, or more, different epitopes on one biological molecule or is capable of specifically binding to epitopes on two, or more, different biological molecules). In some embodiments, an antigen-binding domain of a multispecific antibody (such as a bispecific antibody) comprises two VH/VL units, wherein a first VH/VL unit specifically binds to a first epitope and a second VH/VL unit specifically binds to a second epitope, wherein each VH/VL unit comprises a heavy chain variable domain (VH) and a light chain variable domain (VL). Such multispecific antibodies include, but are not limited to, full length antibodies, antibodies having two or more VL and VH domains. A VH/VL unit that further comprises at least a portion of a heavy chain constant region and/or at least a portion of a light chain constant region may also be referred to as a “half antibody.” In some embodiments, a half antibody comprises at least a portion of a single heavy chain variable region and at least a portion of a single light chain variable region. In some such embodiments, a bispecific antibody that comprises two half antibodies and binds to two antigens comprises a first half antibody that binds to the first antigen or first epitope but not to the second antigen or second epitope and a second half antibody that binds to the second antigen or second epitope and not to the first antigen or first epitope. In some embodiments, a half antibody comprises a sufficient portion of a heavy chain variable region to allow intramolecular disulfide bonds to be formed with a second half antibody. In some embodiments, a half antibody comprises a knob mutation or a hole mutation, for example, to allow heterodimerization with a second half antibody that comprises a complementary hole mutation or knob mutation. Knob mutations and hole mutations are discussed further below.

A “bispecific antibody” is a multispecific antibody comprising an antigen-binding domain that is capable of specifically binding to two different epitopes on one biological molecule or is capable of specifically binding to epitopes on two different biological molecules. A bispecific antibody may also be referred to herein as having “dual specificity” or as being “dual specific.” Unless otherwise indicated, the order in which the antigens bound by a bispecific antibody are listed in a bispecific antibody name is arbitrary. In some embodiments, a bispecific antibody comprises two half antibodies, wherein each half antibody comprises a single heavy chain variable region and a single light chain variable region, and wherein the first half antibody binds to a first antigen and not to a second antigen and the second half antibody binds to the second antigen and not to the first antigen.

The term “knob-into-hole” or “KiH” technology as used herein refers to the technology directing the pairing of two polypeptides together in vitro or in vivo by introducing a protuberance (knob) into one polypeptide and a cavity (hole) into the other polypeptide at an interface in which they interact. For example, KiHs have been introduced in the Fc:Fc binding interfaces, CL:CH1 interfaces or VH/VL interfaces of antibodies (see, e.g., US 2011/0287009, US 2007/0178552, WO 96/027011, WO 98/050431, and Zhu et al., 1997, Protein Science 6:781-788). In some embodiments, KiHs drive the pairing of two different heavy chains together during the manufacture of multispecific antibodies. For example, multispecific antibodies having KiH in their Fc regions can further comprise single variable domains linked to each Fc region, or further comprise different heavy chain variable domains that pair with similar or different light chain variable domains. KiH technology can also be used to pair two different receptor extracellular domains together or any other polypeptide sequences that comprises different target recognition sequences (e.g., including affibodies, peptibodies and other Fc fusions).

The term “knob mutation” as used herein refers to a mutation that introduces a protuberance (knob) into a polypeptide at an interface in which the polypeptide interacts with another polypeptide. In some embodiments, the other polypeptide has a hole mutation (see e.g., U.S. Pat. Nos. 5,731,168, 5,807,706, 5,821,333, 7,695,936, 8,216,805, each incorporated herein by reference in its entirety).

The term “hole mutation” as used herein refers to a mutation that introduces a cavity (hole) into a polypeptide at an interface in which the polypeptide interacts with another polypeptide. In some embodiments, the other polypeptide has a knob mutation (see e.g., U.S. Pat. Nos. 5,731,168, 5,807,706, 5,821,333, 7,695,936, 8,216,805, each incorporated herein by reference in its entirety).

The term “monoclonal antibody” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. In addition to their specificity, the monoclonal antibodies are advantageous in that they are uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the methods provided herein may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA 81:6851-6855 (1984)). Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences (U.S. Pat. No. 5,693,780).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence, except for FR substitution(s) as noted above. The humanized antibody optionally also will comprise at least a portion of an immunoglobulin constant region, typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

In certain embodiments, the bispecific antibody is selected form the group of bispecific antibodies consisting of
a domain exchanged 1+1 bispecific antibody (CrossMab) (a bispecific, full-length IgG antibody comprising a pair of a first light chain and a first heavy chain comprising a first Fab fragment and a pair of a second light chain and a second heavy chain comprising a second Fab fragment,
   wherein in the first Fab fragment
   a) only the CH1 and CL domains are replaced by each other (i.e. the light chain of the first Fab fragment comprises a VL and a CH1 domain and the heavy chain of the first Fab fragment comprises a VH and a CL domain); b) only the VH and VL domains are replaced by each other (i.e. the light chain of the first Fab fragment comprises a VH and a CL domain and the heavy chain of the first Fab fragment comprises a VL and a CH1 domain); or
   c) the CH1 and CL domains and the VH and VL domains are replaced by each other (i.e. the light chain of the first Fab fragment comprises a VH and a CH1 domain and the heavy chain of the first Fab fragment comprises a VL and a CL domain);
   wherein the second Fab fragment comprises a light chain comprising a VL and a CL domain, and a heavy chain comprising a VH and a CH1 domain;
   wherein the first heavy chain and the second heavy chain both comprise a CH3 domain, wherein both CH3 domains are engineered in a complementary manner by respective amino acid substitutions, in order to support heterodimerization of the first heavy chain and the second heavy chain, (in one preferred embodiment, one CH3 domain comprises the knob-mutation and the respective other CH3 domain comprises the hole-mutations);
C-terminal fused 2+1 bispecific antibody (2+1 C-format)
   (a bispecific, full length IgG antibody comprising
   a) one full length antibody comprising two pairs each of a full length antibody light chain and a full length antibody heavy chain, wherein the binding sites formed by each of the pairs of the full length heavy chain and the full length light chain specifically bind to a first antigen, and
   b) one additional binding domain, e.g. a receptor ligand, wherein the additional binding domain is fused to the C-terminus of one heavy chain of the full length antibody;
N-terminal Fab-domain inserted 2+1 bispecific antibody (2+1 N format; TCB) (a bispecific, full-length antibody with additional heavy chain N-terminal binding site with domain exchange comprising
   a first and a second Fab fragment, wherein each binding site of the first and the second Fab fragment specifically bind to a first antigen,
   a third Fab fragment, wherein the binding site of the third Fab fragment specifically binds to a second antigen, and wherein the third Fab fragment comprises a domain crossover such that the variable light chain domain (VL) and the variable heavy chain domain (VH) are replaced by each other, and
   an Fc-region comprising a first Fc-region polypeptide and a second Fc-region polypeptide,
   wherein the first and the second Fab fragment each comprise a heavy chain fragment and a full-length light chain,
   wherein the C-terminus of the heavy chain fragment of the first Fab fragment is fused to the N-terminus of the first Fc-region polypeptide,
   wherein the C-terminus of the heavy chain fragment of the second Fab fragment is fused to the N-terminus of the variable light chain domain of the third Fab fragment and the C-terminus of the CH1 domain of the third Fab fragment is fused to the N-terminus of the second Fc-region polypeptide).

The term “domain crossover” as used herein denotes that in a pair of an antibody heavy chain VH-CH1 fragment and its corresponding cognate antibody light chain, i.e. in an antibody Fab (fragment antigen binding), the domain sequence deviates from the sequence in a native antibody in that at least one heavy chain domain is substituted by its corresponding light chain domain and vice versa. There are three general types of domain crossovers, (i) the crossover of the CH1 and the CL domains, which leads by the domain crossover in the light chain to a VL-CH1 domain sequence and by the domain crossover in the heavy chain fragment to a VH-CL domain sequence (or a full length antibody heavy chain with a VH-CL-hinge-CH2-CH3 domain sequence), (ii) the domain crossover of the VH and the VL domains, which leads by the domain crossover in the light chain to a VH-CL domain sequence and by the domain crossover in the heavy chain fragment to a VL-CH1 domain sequence, and (iii) the domain crossover of the complete light chain (VL-CL) and the complete VH-CH1 heavy chain fragment (“Fab crossover”), which leads to by domain crossover to a light chain with a VH-CH1 domain sequence and by domain crossover to a heavy chain fragment with a VL-CL domain sequence (all aforementioned domain sequences are indicated in N-terminal to C-terminal direction).

As used herein the term “replaced by each other” with respect to corresponding heavy and light chain domains refers to the aforementioned domain crossovers. As such, when CH1 and CL domains are “replaced by each other” it is referred to the domain crossover mentioned under item (i) and the resulting heavy and light chain domain sequence. Accordingly, when VH and VL are “replaced by each other” it is referred to the domain crossover mentioned under item (ii); and when the CH1 and CL domains are “replaced by each other” and the VH and VL domains are “replaced by each other” it is referred to the domain crossover mentioned under item (iii).

In certain embodiments the Fc-region containing polypeptide or antibody is a bispecific antibody or an Fc-fusion protein.

In the method according to the invention it was shown that only for hydrophilic mabs (retention time_{mab}≤retention time_{rituximab}) an improved HMW removal was achieved by addition of an antichaotropic salt to the load solution compared to a load solution having the same conductivity in the absence of an antichaotropic salt. In contrast to that, for hydrophobic mabs (retention time_{mab}>retention time_{rituximab}) no positive impact was observed with the addition of an antichaotropic salt.

The term “hydrophilic antibody” according to the invention denotes an antibody that has a retention time on hydrophobic interaction chromatography (HIC) column that is equal or less than the HIC retention time of rituximab on the same HIC column and under the same chromatography conditions. Likewise, a “hydrophobic antibody” according to the invention denotes an antibody that has a retention time on hydrophobic interaction chromatography (HIC) column that is more than the HIC retention time of rituximab on the same HIC column and under the same chromatography conditions. In other words, mabs with a retention time≤retention time_{rituximab}, i.e. that have the same or a shorter retention time as rituximab, are defined to be hydrophilic, mabs with retention time>retention time_{rituximab}, i.e. that have a longer retention time than that of rituximab, are defined to be hydrophobic.

The method for determination of the retention times is described in point 10 of the materials and methods section. The retention times of the mabs determined with this method were in the range from 19 min. to 41 min. An overview of the retention times of the mabs is given in Table MM-1. The retention time of Rituximab was found to be the cut-point for defining hydrophilic and hydrophobic mabs.

In certain embodiments of the above aspects and the other embodiments the hydrophilic antibody is an antibody that has a retention time on a hydrophobic interaction chromatography (HIC) material that is equal or less than that of rituximab (on the same HIC material and under the same operating conditions).

In certain embodiments of the above aspects and the other embodiments the hydrophobic interaction chromatography (HIC) material contains polyether groups (ethyl ether groups) as ligand. In certain embodiments of the above aspects and the other embodiments the hydrophobic interaction chromatography (HIC) material contains polyether groups with the following structure (—(OCH₂CH2)ₙOH) as ligand. In certain embodiments of the above aspects and the other embodiments the hydrophobic interaction chromatography (HIC) material contains a polymethacrylate base material/matrix. In a preferred embodiment of the above aspects and the other embodiments the hydrophobic interaction chromatography (HIC) material contains polyether groups (ethyl ether groups) as ligand, has a mean pore size of 100 nm and a particle size of 10 μm. In certain embodiments of the above aspects and the other embodiments the hydrophobic interaction chromatography (HIC) material is a TSKgel® Ether-5 PW chromatography material. In one preferred embodiment of the above aspects and the other embodiments the retention time on the HIC chromatography material is determined using the HIC chromatography material (of any one of the other five embodiments above), and with a column length of 75 mm, and with an inner diameter of 7.5 mm, and with an elution buffer gradient at a flow rate of 8.8 ml/min, and wherein the antibody is applied to the chromatography material at a concentration of 1 mg/ml.

The skilled person knows how to determine a buffer gradient for the elution of the given antibody. A suitable elution buffer gradient is described herein in point 10 of the Material and Methods section (Determination of retention time and hydrophobicity), especially in point 10.8.

“Loading density” or “loading capacity” or “load density” or “load capacity” or “loaded amount” which terms are used interchangeably herein refers to the amount, e.g. grams, of antibody or protein brought in contact with a volume of chromatography material, e.g. liters. In some examples, loading density is expressed in g/L.

In a preferred embodiment of the above aspects and the other embodiments the load amount of the MM HIC/IEX chromatography material (i.e. the mixed mode chromatography material that comprises ion exchange and hydrophobic interaction functional groups) is 10 g/L or higher, i.e. it is 10 g of protein per Liter of chromatography material (10 g/L) or higher. In certain embodiments of the above aspects and the other embodiments the load amount of the MM HIC/IEX chromatography material is 15 g/L or higher. In certain embodiments of the above aspects and the other embodiments the load amount of the MM HIC/IEX chromatography material is 20 g/L or higher. In certain embodiments of the above aspects and the other embodiments the load amount of the MM HIC/IEX chromatography material is 30 g/L or higher. In certain embodiments of the above aspects and the other embodiments the load amount of the MM HIC/IEX chromatography material is 40 g/L or higher In certain embodiments of the above aspects and the other embodiments the load amount of the MM HIC/IEX chromatography material is 50 g/L or higher.

In certain embodiments of the above aspects and the other embodiments the load amount of the MM HIC/IEX chromatography material (i.e. the mixed mode chromatography material that comprises ion exchange and hydrophobic interaction functional groups) is from (and including) 10 g/L to 650 g/L, i.e. it is from (and including) 10 g of protein per Liter of chromatography material (10 g/L) to 650 g of protein per Liter of chromatography material (650 g/L). In certain embodiments of the above aspects and the other embodiments of the above aspects and the other embodiments the load amount of the MM HIC/IEX chromatography material is from (and including) 30 g/L to 600 g/L. In certain embodiments of the above aspects and the other embodiments the load amount of the MM HIC/IEX chromatography material is from (and including) 50 g/L to 500 g/L. In certain embodiments of the above aspects and the other embodiments the load amount of the MM HIC/IEX chromatography material is from (and including) 50 g/L to 400 g/L. In a preferred embodiment of the above aspects and the other embodiments the load amount of the MM HIC/IEX chromatography material is from (and including) 15 g/L to 350 g/L.

It must be noted that as used herein and in the appended claims, the singular forms “a”, “an”, and “the” include plural reference unless the context clearly dictates otherwise. Thus, for example, reference to “a salt” includes a plurality of such salts, for example one to three, or one to two. As well, the terms “a” (or “an”), “one or more” and “at least one” can be used interchangeably herein.

The term “about” denotes that the thereafter following value is no exact value but is the center point of a range that is +/−10% of the value, or +/−5% of the value, or +/−2% of the value, or +/−1% of the value. If the value is a relative value given in percentages the term “about” also denotes that the thereafter following value is no exact value but is the center point of a range that is +/−10% of the value, or +/−5% of the value, or +/−2% of the value, or +/−1% of the value, whereby the upper limit of the range cannot exceed a value of 100%.

The term “cell” or “host cell” refers to a cell into which a nucleic acid, e.g. encoding a heterologous polypeptide, can be or is transfected. The term “cell” includes both prokaryotic cells, which are used for expression of a nucleic acid and production of the encoded polypeptide including propagation of plasmids, and eukaryotic cells, which are used for the expression of a nucleic acid and production of the encoded polypeptide. In one embodiment, the eukaryotic cells are mammalian cells. In one embodiment the mammalian cell is a CHO cell, optionally a CHO K₁ cell (ATCC CCL-61 or DSM ACC 110), or a CHO DG44 cell (also known as CHO-DHFR [-], DSM ACC 126), or a CHO XL99 cell, a CHO-T cell (see e.g. Morgan, D., et al., Biochemistry 26 (1987) 2959-2963), or a CHO-S cell, or a Super-CHO cell (Pak, S.C.O., et al. Cytotechnology 22 (1996) 139-146). If these cells are not adapted to growth in serum-free medium or in suspension an adaptation prior to the use in the current method is to be performed. As used herein, the expression “cell” includes the subject cell and its progeny. Thus, the words “transformant” and “transformed cell” include the primary subject cell and cultures derived there from without regard for the number of transfers or subcultivations. It is also understood that all progeny may not be precisely identical in DNA content, due to deliberate or inadvertent mutations. Variant progeny that have the same function or biological activity as screened for in the originally transformed cell are included.

The term “Fc-region” denotes the part of an immunoglobulin that is not involved directly in binding to the immunoglobulin's binding partner, but exhibit various effector functions. Depending on the amino acid sequence of the constant region of the heavy chains, immunoglobulins are divided in the classes: IgA, IgD, IgE, IgG, and IgM. Some of these classes are further divided into subclasses (isotypes), i.e. IgG in IgG1, IgG2, IgG3, and IgG4, or IgA in IgA1 and IgA2. According to the class to which an immunoglobulin belongs the heavy chain constant regions of immunoglobulins are called α (IgA), δ (IgD), ε (IgE), γ (IgG), and μ (IgM), respectively.

The term “Fc-region” is used herein to define a C-terminal region fragment of an immunoglobulin heavy chain that contains at least a portion of the constant region. The term includes native sequence Fc-regions and variant Fc-regions. In one embodiment, a human IgG heavy chain Fc-region extends from Cys226, or from Pro230, to the carboxyl-terminus of the heavy chain. However, the C-terminal lysine (Lys447) or glycine-lysine dipeptide (Gly446-Lys447), respectively, of the Fc-region may or may not be present. Numbering according to Kabat EU index.

An “Fc-region” of an immunoglobulin is a term well known to the skilled artisan and defined on basis of the papain cleavage of full length immunoglobulins.

The antibodies described herein always contain an Fc-region, thus the antibodies as reported herein are Fc-region containing polypeptides or antibodies.

The term “constant region (of an antibody)” is used herein to define the part of an immunoglobulin heavy chain excluding the variable domain.

The term “antibody-related high molecular weight (HMW) impurity” refers to an impurity which has about the molecular weight of a dimer (of the same desired antibody/target molecule monomer that is being produced or purified) or a higher molecular weight.

In certain embodiments of the above aspects and the other embodiments the antibody-related high molecular weight (HMW) impurity has a molecular weight of about 250 kDa or more. In a preferred embodiment of the above aspects and the other embodiments the antibody-related high molecular weight (HMW) impurity has a molecular weight of about 285 kDa or more. In certain embodiments the antibody-related high molecular weight (HMW) impurity has a molecular weight of about 300 kDa or more. In preferred embodiments the antibody-related high molecular weight (HMW) impurity is at least a dimer, or a trimer, or any multimer of the desired antibody/target molecule. Thus, in certain embodiments the antibody-related high molecular weight (HMW) impurity is an impurity which has about the molecular weight of a dimer of the same antibody or a higher molecular weight. Further, it may include fragments (like half-antibodies) of the desired antibody/target molecule as well. For example, the antibody-related high molecular weight (HMW) impurity is a dimer or a trimer plus a fragment of the target antibody.

Methods of measuring HMW impurities are known in the art and are described in, e.g., WO 2011/150110. Such methods include, e.g., size exclusion chromatography, capillary electrophoresis-sodium dodecyl sulfate (CE-SDS) and liquid chromatography-mass spectrometry (LC-MS).

HMW impurities may be determined as described in the Examples section.

The term “viral impurity” or “viral impurity content” refers to an impurity by viruses or viral particles. For practical and safety reasons viral impurity contaminations are analysed with retrovirus like particles (RVLPs) as surrogates for actual viruses/viral impurities. RVLPs in turn can be determined by the determination of the RNA content (e.g. by quantitative reverse transcriptase (RT) PCR).

In preferred embodiments of the above aspects and the other embodiments
the method is for producing an antibody composition with reduced antibody-related high molecular weight (HMW) impurity content and/or with reduced viral impurity content,
the antibody is applied to the MM HIC/IEX in a solution comprising the antibody, at least one antibody-related HMW impurity and/or at least one viral impurity and an antichaotropic salt,
the antibody composition with reduced antibody-related HMW impurity content and/or with reduced viral impurity content is recovered from the flowthrough, and
thereby an antibody composition with reduced antibody-related HMW impurity content and/or with reduced viral impurity content is produced.

In preferred embodiments of the above aspects and the other embodiments
the method is for producing an antibody composition with reduced antibody-related high molecular weight (HMW) impurity content and with reduced viral impurity content,
the antibody is applied to the MM HIC/IEX in a solution comprising the antibody, at least one antibody-related HMW impurity and at least one viral impurity and an antichaotropic salt,
the antibody composition with reduced antibody-related HMW impurity content and with reduced viral impurity content is recovered from the flowthrough, and
thereby an antibody composition with reduced antibody-related HMW impurity content and with reduced viral impurity content is produced.

In preferred embodiments of the aspects and the other embodiments
the method is for producing an antibody composition with reduced antibody-related high molecular weight (HMW) impurity content,
the antibody is applied to the MM HIC/IEX in a solution comprising the antibody, at least one antibody-related HMW impurity and an antichaotropic salt,
the antibody composition with reduced antibody-related HMW impurity content is recovered from the flowthrough, and
thereby an antibody composition with reduced antibody-related HMW impurity content is produced.

In certain embodiments of the aspects and the other embodiments the antibody-related (HMW) impurity content is reduced compared to the solution applied to the MM HIC/IEX in step b). In a preferred embodiment of the above aspects and the other embodiments the antibody-related (HMW) impurity content is reduced compared to a solution essentially without an antichaotropic salt; and/or compared to a solution comprising a hydrophobic antibody.

In preferred embodiments of the above aspects and the other embodiments
the method is for producing an antibody composition with reduced viral impurity content/viral impurities,
the antibody is applied to the MM HIC/IEX in a solution comprising the antibody, at least one viral impurity and an antichaotropic salt,
the antibody composition with reduced viral impurity content/viral impurities is recovered from the flowthrough, and
thereby an antibody composition with reduced viral impurity content/viral impurities is produced.

In certain embodiments of the above aspects and the other embodiments the viral impurity content/viral impurities is reduced compared to the solution applied to the MM HIC/IEX in step b).

In certain embodiments of the above aspects and the other embodiments the viral impurity content/viral impurities is reduced compared to a solution essentially without an antichaotropic salt; and/or compared to a solution comprising a hydrophobic antibody.

As used herein, the terms “mixed mode chromatography” or “mixed mode chromatography material” or “MM HIC/IEX” refer to a mixed mode or multimodal (MM) chromatography material (the terms “mixed mode” and “multimodal” can be used interchangeably) that comprises a hydrophobic interaction (HIC) functionality/part, and an ion exchange (IEX) functionality/part. In other words, the mixed mode chromatography material comprises ion exchange functional groups and hydrophobic interaction functional groups. It thus combines at least two functionalities in one chromatography material. The MMIEX chromatography material may additionally include other functionalities e.g. hydrogen bonding interactions.

In certain embodiments of the above aspects and the other embodiments the MM HIC/IEX comprises anion exchange functional groups or cation exchange functional groups (as ion exchange functional groups); in addition to the hydrophobic interaction functional groups. In certain embodiments of the above aspects and the other embodiments the MM HIC/IEX comprises anion exchange functional groups (as ion exchange functional groups) This material then combines mainly anion exchange (AEX) and hydrophobic interaction functionalities (HIC). In a preferred embodiment of the above aspects and the other embodiments the MM HIC/IEX comprises strong anion exchange functional groups (as ion exchange functional groups) (i.e. it is a multimodal strong anion exchange chromatography material). In certain embodiments of the above aspects and the other embodiments the MM HIC/IEX comprises a charged nitrogen atom and a ring structure (as functional groups). In certain embodiments of the above aspects and the other embodiments the MM HIC/IEX comprises a quaternary amine (as ion exchange functional groups). In certain embodiments of the above aspects and the other embodiments the MM HIC/IEX comprises a quaternary amine (as ion exchange functional groups) and highly crosslinked agarose (as matrix). In certain embodiments of the above aspects and the other embodiments the MM HIC/IEX comprises N-Benzyl-N-methyl ethanol amine (as functional groups) and highly crosslinked agarose (as matrix). In certain embodiments of the above aspects and the other embodiments the MM HIC/IEX is Capto™ adhere ImpRes, Capto™ Adhere or Nuvia aPrime4A. In a preferred embodiment of the above aspects and the other embodiments the MM HIC/IEX is Capto™ adhere ImpRes or Capto™ Adhere.

In certain embodiments of the above aspects and the other embodiments the MM HIC/IEX comprises cation exchange functional groups (as ion exchange functional groups). This material then combines mainly cation exchange (CEX) and hydrophobic interaction (HIC) functionalities. In a preferred embodiment of the above aspects and the other embodiments the MM HIC/IEX comprises weak cation exchange functional groups (as ion exchange functional groups) (i.e. it is a multimodal weak cation exchange chromatography material). In certain embodiments of the above aspects and the other embodiments the MM HIC/IEX comprises weak cation exchange functional groups (as ion exchange functional groups) and highly crosslinked agarose (as matrix). In certain embodiments of the above aspects and the other embodiments the MM HIC/IEX comprises N-benzoylhomocysteine (as ion exchange functional groups) and highly crosslinked agarose (as matrix). In one preferred embodiment of the above aspects and the other embodiments the MM HIC/IEX is Capto™ MMC or Capto™ MMC ImpRes. In certain embodiments of the above aspects and the other embodiments the MM HIC/IEX is Capto™ MMC ImpRes.

The term “antichaotropic salt” (or kosmotropic salt) refers to chemical compounds which are capable of making a protein conformation less water soluble. Antichaotropic agents decrease the entropy of the system by interfering with intramolecular interactions mediated by non covalent forces that contribute to the stability and structure of water-water interactions. Antichaotropic salts typically cause water molecules to favorably interact, which also stabilizes intermolecular interactions in macromolecules. Antichaotropic salts can be ionic and/or nonionic. Examples of such antichaotropic agents include but are not limited to Na₂SO₄, KCl, (NH₄)₂SO₄, K₂SO₄ or NaCl etc.

In certain embodiments of the above aspects and the other embodiments reported herein the antichaotropic salt is selected from the group consisting of chlorides, sulfates, citrates, carbonates, phosphates, acetates or fluorides. In one embodiment the antichaotropic salt is selected from the group consisting of chlorides, sulfates, citrates, phosphates or acetates. In one embodiment the antichaotropic salt is selected from the group consisting of chlorides or sulfates. In one embodiment the antichaotropic salt is selected from the group consisting of chlorides, sulfates, citrates, carbonates, phosphates, acetates or fluorides which comprise calcium (Ca), sodium (Na), ammonium (NH4) or potassium (K). In certain embodiments of the above aspects and the other embodiments the antichaotropic salt is a calcium-, sodium-, ammonium- or potassium-salt.

In one preferred embodiment the antichaotropic salt is a sodium-, ammonium- or potassium-salt. In one embodiment the antichaotropic salt is selected from the group consisting of chlorides, sulfates, citrates, phosphates or acetates which comprise calcium (Ca), sodium (Na), ammonium (NH4) or potassium (K). In one embodiment the antichaotropic salt is a calcium-, sodium-, ammonium- or potassium-salt. In one preferred embodiment the antichaotropic salt is a sodium-, ammonium- or potassium-salt. In one embodiment the antichaotropic salt is selected from the group consisting of chlorides or sulfates which comprise calcium (Ca), sodium (Na), ammonium (NH4) or potassium (K). In one embodiment the antichaotropic salt is a calcium-, sodium-, ammonium- or potassium-salt. In one preferred embodiment the antichaotropic salt is a sodium-, ammonium- or potassium-salt. In one embodiment the antichaotropic salt is selected from the group consisting of chlorides or sulfates which comprise sodium (Na), ammonium (NH4) or potassium (K). In one preferred embodiment the antichaotropic salt is a sodium-, ammonium- or potassium-salt. In one embodiment the antichaotropic salt is selected from the group consisting of (NH₄)₂SO₄, Na₂SO₄, K₂SO₄, NaCl, KCl and CaCl₂). In a preferred embodiment of the above aspects and the other embodiments the antichaotropic salt is selected from the group consisting of (NH₄)₂SO₄, Na₂SO₄, K₂SO₄, NaCl and KCl. In one embodiment the antichaotropic salt is selected from the group consisting of (NH₄)₂SO₄, Na₂SO₄ and K₂SO₄. In one preferred embodiment the antichaotropic salt is Na₂SO₄.

In certain embodiments of the above aspects and the other embodiments the antichaotropic salt has a molar surface tension increment in the range of and including 1.285 to 4.183×10E3 dyn*g*cm⁻¹*mol⁻¹. In a preferred embodiment the antichaotropic salt has a molar surface tension increment in the range of and including 1.3 to 3.0×10E3 dyn*g*cm⁻¹*mol⁻¹. In a further preferred embodiment the antichaotropic salt has a molar surface tension increment in the range of and including 1.46 to 2.86×10E3 dyn*g*cm 1*mol⁻¹.

The skilled person knows how to determine the molar surface tension increment. Information in this regard can be found e.g. in Laurel M. Pegram and M. Thomas Record, Jr., J. Phys. Chem. B 2007, 111, 5411-5417 and Jan-Christer Janson; Hydrophobic Interaction Chromatography, p. 170, table 6.2

The term “flowthrough”, “flowthrough mode”, “operated in flowthrough mode” or a similar expression refers to a way of performing or operating a chromatography such that the conditions (e.g. pH, buffer content and concentration, conductivity, etc.) of the chromatography are chosen in a way that the protein or antibody of interest does not significantly bind to the chromatography material. Instead the protein or antibody of interest flows through the chromatography material. As essentially no binding occurs, it is also not necessary that an elution takes place to release the protein or antibody of interest from the chromatography material (as it is the case in bind-and-elute operation mode). It may be beneficial to perform an additional step of rinsing the chromatography material (e.g. with the equilibration buffer or a similar solution) after fully loading (i.e. applying the protein of interest) the chromatography material with the solution comprising the protein or antibody of interest to recover residual protein or antibody of interest that is still in the chromatography column. Thus, if a chromatography material is “operated in flowthrough mode” this includes the steps of applying the solution to be purified or produced that comprises the antibody/protein of interest; flowing the antibody/protein of interest through the chromatography material (and thereby purifying the antibody/protein of interest by separating the antibody/protein of interest from impurities); and recovering the antibody/protein of interest in the flowthrough (fraction). Optionally a rinsing step can be performed.

The skilled person knows how the chromatography conditions have to be chosen to operate in flowthrough mode. For example, to achieve flowthrough conditions the skilled person understands that the pH must be chosen in a way that-depending on the pI (isoelectric point) of the molecule—the molecule of interest does not significantly bind to the chromatography material. If the pH is lower than the pI of the molecule, the molecule is positively charged and would not bind significantly to a mixed mode chromatography material with anion exchange functional groups. On the other hand, if the pH is higher than that of the pI of the molecule, the molecule is negatively charged and would not significantly bind to a mixed mode chromatography material with cation exchange functional groups.

The skilled person knows that chromatography conditions can be influenced by the value of the pH. As described above the choice of the pH value largely depends on the pI of the molecule and the conditions that are to be achieved.

In one embodiment of all aspects the solution comprising the antibody and an antichaotropic salt (of step b) has a pH value of 4.0 or higher. In one embodiment the solution comprising the antibody and an antichaotropic salt (of step b) has a pH value of 4.5 or higher. In one embodiment the solution comprising the antibody and an antichaotropic salt (of step b) has a pH value of 5.0 or higher. In one embodiment the solution comprising the antibody and an antichaotropic salt (of step b) has a pH value of 5.5 or higher. In one preferred embodiment the solution comprising the antibody and an antichaotropic salt (of step b) has a pH value of 6.0 or higher. In one embodiment the solution comprising the antibody and an antichaotropic salt (of step b) has a pH value of 7.0 or higher.

In certain embodiments of the above aspects and the other embodiments the solution comprising the antibody and an antichaotropic salt (of step b) has a pH value of from 3.5 to 9.5. In one preferred embodiment the solution comprising the antibody and an antichaotropic salt (of step b) has a pH value of from 4.0 to 9.0. In one embodiment the solution comprising the antibody and an antichaotropic salt (of step b) has a pH value of from 5.0 to 8.5. In one preferred embodiment the solution comprising the antibody and an antichaotropic salt (of step b) has a pH value of from 5.5 to 8.5. In one embodiment the solution comprising the antibody and an antichaotropic salt (of step b) has a pH value of from 5.5 to 8.0. In one embodiment the solution comprising the antibody and an antichaotropic salt (of step b) has a pH value of from 5.0 to 8.0.

The skilled person knows that chromatography conditions can be influenced by the conductivity conditions.

In certain embodiments of the above aspects and the other embodiments the solution comprising the antibody and an antichaotropic salt has a conductivity of 0.5 mS/cm or higher. In one preferred embodiment the solution comprising the antibody and an antichaotropic salt has a conductivity of from (and including) 0.5 to 120 mS/cm. In one embodiment the solution comprising the antibody and an antichaotropic salt has a conductivity of from (and including) 0.5 to 100 mS/cm. In one embodiment the solution comprising the antibody and an antichaotropic salt has a conductivity of from (and including) 0.5 to 80 mS/cm. In one embodiment the solution comprising the antibody and an antichaotropic salt has a conductivity of from (and including) 0.5 to 60 mS/cm. In one embodiment the solution comprising the antibody and an antichaotropic salt has a conductivity of from (and including) 0.5 to 50 mS/cm. In one embodiment the solution comprising the antibody and an antichaotropic salt has a conductivity of from (and including) 0.5 to 30 mS/cm. In one preferred embodiment the solution comprising the antibody and an antichaotropic salt has a conductivity of from (and including) 4 to 25 mS/cm. In one embodiment the solution comprising the antibody and an antichaotropic salt has a conductivity of from (and including) 10 to 20 mS/cm. In particular with respect to viral impurity removal in one embodiment the solution comprising the antibody and an antichaotropic salt has a conductivity of from (and including) 5 to 25 mS/cm. In one embodiment the solution comprising the antibody and an antichaotropic salt has a conductivity of from (and including) 8 to 22 mS/cm. In one embodiment the solution comprising the antibody and an antichaotropic salt has a conductivity of from (and including) 10 to 20 mS/cm.

In certain embodiments of the above aspects and the other embodiments in the solution comprising the antibody and an antichaotropic salt the antichaotropic salt has a molar concentration of the antichaotropic salt of 5 mM or higher. In one preferred embodiment in the solution comprising the antibody and an antichaotropic salt the antichaotropic salt has a molar concentration of the antichaotropic salt of 10 mM or higher. In one embodiment in the solution comprising the antibody and an antichaotropic salt the antichaotropic salt has a molar concentration of the antichaotropic salt of 20 mM or higher. In one embodiment in the solution comprising the antibody and an antichaotropic salt the antichaotropic salt has a molar concentration of the antichaotropic salt of from (and including) 5 mM to 1000 mM. In one preferred embodiment in the solution comprising the antibody and an antichaotropic salt the antichaotropic salt has a molar concentration of the antichaotropic salt of from (and including) 10 mM to 900 mM. In one embodiment in the solution comprising the antibody and an antichaotropic salt the antichaotropic salt has a molar concentration of the antichaotropic salt of from 15 mM to 850 mM. In particular with respect to viral impurity removal in one embodiment the solution comprising the antibody and an antichaotropic salt has a molar concentration of the antichaotropic salt of from (and including) 50 mM to 400 mM. In one embodiment the solution comprising the antibody and an antichaotropic salt has a molar concentration of the antichaotropic salt of from (and including) 50 mM to 300 mM. In one embodiment the solution comprising the antibody and an antichaotropic salt has a molar concentration of the antichaotropic salt of from (and including) 50 mM to 250 mM.

It is understood that the antichaotropic salt can be added to the solution that is loaded onto the MM HIC/IEX and/or it can be present in the solution loaded onto the MM HIC/IEX from method steps that were performed prior to the MM HIC/IEX. For example, the antichaotropic salt could have been present in a solution in an earlier chromatography step.

The following examples and figures are provided to aid the understanding of the present invention, the true scope of which is set forth in the appended claims. It is understood that modifications can be made in the procedures set forth without departing from the spirit of the invention.

### DESCRIPTION OF THE FIGURES

FIG. 1 HMW removal value [%] of flowthrough fractions with increasing total loaded amount [mgₚᵣₒₜᵢₑₙ/mL_{chromatography medium}] of the hydrophilic mab1 in 1.5 M Tris/Acetate buffer compared to 70 mM Tris/Acetate buffers containing the antichaotropic salts Na₂SO₄, KCl, (NH₄)₂SO₄, K₂SO₄ or NaCl in flowthrough mode on MMAEX Capto™ adhere ImpRes RCs for a load condition of pH 8 and a load conductivity of 20 mS/cm.

FIG. 2 HMW removal value [%] of flowthrough fractions with increasing total loaded amount [mgₚᵣₒₜᵢₑₙ/mL_{chromatography medium}] of the hydrophilic mab2 in 1.5 M Tris/Acetate buffer compared to 70 mM Tris/Acetate buffers containing the antichaotropic salts Na₂SO₄, KCl, (NH₄)₂SO₄, K₂SO₄ or NaCl in flowthrough mode on MMAEX Capto™ adhere ImpRes RCs for a load condition of pH 8 and a load conductivity of 20 mS/cm.

FIG. 3 HMW removal value [%] of flowthrough fractions with increasing total loaded amount [mgₚᵣₒₜᵢₑₙ/mL_{chromatography medium}] of the hydrophilic mab4 in 1.5 M Tris/Acetate buffer compared to 70 mM Tris/Acetate buffers containing the antichaotropic salts Na₂SO₄, KCl, (NH₄)₂SO₄, K₂SO₄ or NaCl in flowthrough mode on MMAEX Capto™ adhere ImpRes RCs for a load condition of pH 8 and a load conductivity of 20 mS/cm.

FIG. 4 HMW removal value [%] of flowthrough fractions with increasing total loaded amount [mgₚᵣₒₜᵢₑₙ/mL_{chromatography medium}] of the hydrophilic mab5 in 1.5 M Tris/Acetate buffer compared to 70 mM Tris/Acetate buffers containing the antichaotropic salts Na₂SO₄, KCl, (NH₄)₂SO₄, K₂SO₄ or NaCl in flowthrough mode on MMAEX Capto™ adhere ImpRes RCs for a load condition of pH 8 and a load conductivity of 20 mS/cm.

FIG. 5 HMW removal value [%] of flowthrough fractions with increasing total loaded amount [mgₚᵣₒₜᵢₑₙ/mL_{chromatography medium}] of the hydrophobic mab6 in 1.5 M Tris/Acetate buffer compared to 70 mM Tris/Acetate buffers containing the antichaotropic salts Na₂SO₄, KCl, (NH₄)₂SO₄, K₂SO₄ or NaCl in flowthrough mode on MMAEX Capto™ adhere ImpRes RCs for a load condition of pH 8 and a load conductivity of 20 mS/cm.

FIG. 6 HMW removal value [%] of flowthrough fractions with increasing total loaded amount [mgₚᵣₒₜᵢₑₙ/mL_{chromatography medium}] of the hydrophobic mab7 in 1.5 M Tris/Acetate buffer compared to 70 mM Tris/Acetate buffers containing the antichaotropic salts Na₂SO₄, KCl, (NH₄)₂SO₄, K₂SO₄ or NaCl in flowthrough mode on MMAEX Capto™ adhere ImpRes RCs for a load condition of pH 8 and a load conductivity of 20 mS/cm.

FIG. 7 HMW removal value [%] of flowthrough fractions with increasing total loaded amount [mgₚᵣₒₜᵢₑₙ/mL_{chromatography medium}] of the hydrophobic mab8 in 1.5 M Tris/Acetate buffer compared to 70 mM Tris/Acetate buffers containing the antichaotropic salts Na₂SO₄, KCl, (NH₄)₂SO₄, K₂SO₄ or NaCl in flowthrough mode on MMAEX Capto™ adhere ImpRes RCs for a load condition of pH 8 and a load conductivity of 20 mS/cm.

FIG. 8A Introduction of trend lines into FIG. 2 for the load condition 1.5 M Tris/Acetate, pH 8 at a conductivity of 20 mS/cm and for the load condition 70 mM Tris/Acetate, 100 mM (NH₄)₂SO₄. pH 8 at a conductivity of 20 mS/cm to calculate the HMW removal [%] for flowthrough pools of the hydrophilic mab2 on MMAEX Capto™ adhere ImpRes RCs.

FIG. 8B Introduction of trend lines into FIG. 6 for the load condition 1.5 M Tris/Acetate, pH 8 at a conductivity of 20 mS/cm and for the load condition 70 mM Tris/Acetate, 100 mM (NH₄)₂SO₄. PH 8 at a conductivity of 20 mS/cm to calculate the HMW removal [%] for flowthrough pools of the hydrophobic mab7 on MMAEX Capto™ adhere ImpRes RCs.

FIG. 9A Comparison of calculated HMW removal [%] for the flowthrough pools of the hydrophilic mab2 for the load conditions in 1.5 M Tris/Acetate, pH 8 at a conductivity of 20 mS/cm and for the load condition 70 mM Tris/Acetate, 100 mM (NH₄)₂SO₄. PH 8 at a conductivity of 20 mS/cm using the trend lines introduced in FIG. 8A on MMAEX Capto™ adhere ImpRes RCs.

FIG. 9B Comparison of calculated HMW removal [%] for the flowthrough pools of the hydrophobic mab7 for the load conditions in 1.5 M Tris/Acetate, pH 8 at a conductivity of 20 mS/cm and for the load condition 70 mM Tris/Acetate, 100 mM (NH₄)₂SO₄. pH 8 at a conductivity of 20 mS/cm using the trend lines introduced in FIG. 8B on MMAEX Capto™ adhere ImpRes RCs.

FIG. 10 HMW removal values [%] of flowthrough fractions with increasing total loaded amount [mgₚᵣₒₜᵢₑₙ/mL_{chromatography medium}] of the hydrophilic mab1 in 1.0 M Tris/Citrate buffer compared to 70 mM Tris/Citrate buffers containing the antichaotropic salts Na₂SO₄ or KCl in flowthrough mode on MMAEX Capto™ adhere ImpRes RCs for a load condition of pH 6 and a load conductivity of 20 mS/cm.

FIG. 11 HMW removal value [%] of flowthrough fractions with increasing total loaded amount [mgₚᵣₒₜᵢₑₙ/mL_{chromatography medium}] of the hydrophilic mab2 in 1.0 M Tris/Citrate buffer compared to 70 mM Tris/Citrate buffers containing the antichaotropic salts Na₂SO₄ or KCl in flowthrough mode on MMAEX Capto™ adhere ImpRes RCs for a load condition of pH 6 and a load conductivity of 20 mS/cm.

FIG. 12 HMW removal value [%] of flowthrough fractions with increasing total loaded amount [mgₚᵣₒₜᵢₑₙ/mL_{chromatography medium}] of the hydrophilic mab4 in 1.0 M Tris/Citrate buffer compared to 70 mM Tris/Citrate buffers containing the antichaotropic salts Na₂SO₄ or KCl in flowthrough mode on MMAEX Capto™ adhere ImpRes RCs for a load condition of pH 6 and a load conductivity of 20 mS/cm.

FIG. 13 HMW removal value [%] of flowthrough fractions with increasing total loaded amount [mgₚᵣₒₜᵢₑₙ/mL_{chromatography medium}] of the hydrophilic mab5 in 1.0 M Tris/Citrate buffer compared to 70 mM Tris/Citrate buffers containing the antichaotropic salts Na₂SO₄ or KCl in flowthrough mode on MMAEX Capto™ adhere ImpRes RCs for a load condition of pH 6 and a load conductivity of 20 mS/cm.

FIG. 14 HMW removal value [%] of flowthrough fractions with increasing total loaded amount [mgₚᵣₒₜᵢₑₙ/mL_{chromatography medium}] of the hydrophobic mab6 in 1.0 M Tris/Citrate buffer compared to 70 mM Tris/Citrate buffers containing the antichaotropic salts Na₂SO₄ or KCl in flowthrough mode on MMAEX Capto™ adhere ImpRes RCs for a load condition of pH 6 and a load conductivity of 20 mS/cm.

FIG. 15 HMW removal value [%] of flowthrough fractions with increasing total loaded amount [mgₚᵣₒₜᵢₑₙ/mL_{chromatography medium}] of the hydrophobic mab7 in 1.0 M Tris/Citrate buffer compared to 70 mM Tris/Citrate buffers containing the antichaotropic salts Na₂SO₄ or KCl in flowthrough mode on MMAEX Capto™ adhere ImpRes RCs for a load condition of pH 6 and a load conductivity of 20 mS/cm.

FIG. 16 HMW removal value [%] of flowthrough fractions with increasing total loaded amount [mgₚᵣₒₜᵢₑₙ/mL_{chromatography medium}] of the hydrophobic mab8 in 1.0 M Tris/Citrate buffer compared to 70 mM Tris/Citrate buffers containing the antichaotropic salts Na₂SO₄ or KCl in flowthrough mode on MMAEX Capto™ adhere ImpRes RCs for a load condition of pH 6 and a load conductivity of 20 mS/cm.

FIG. 17A HMW removal value [%] of flowthrough fractions with increasing total loaded amount [mgₚᵣₒₜᵢₑₙ/mL_{chromatography medium}] of the hydrophilic mab2 in 300 mM Tris/Citrate buffer compared to 70 mM Tris/Citrate buffers containing the antichaotropic salts Na₂SO₄, KCl, (NH₄)₂SO₄, K₂SO₄ or NaCl in flowthrough mode on MMAEX Capto™ adhere ImpRes RCs for a load condition of pH 6 and a load conductivity of 10 mS/cm.

FIG. 17B HMW removal value [%] of flowthrough fractions with increasing total loaded amount [mgₚᵣₒₜᵢₑₙ/mL_{chromatography medium}] of the hydrophobic mab7 in 300 mM Tris/Citrate buffer compared to 70 mM Tris/Citrate buffers containing the antichaotropic salts Na₂SO₄, KCl, (NH₄)₂SO₄, K₂SO₄ or NaCl in flowthrough mode on MMAEX Capto™ adhere ImpRes RCs for a load condition of pH 6 and a load conductivity of 10 mS/cm.

FIG. 18A HMW removal values [%] of flowthrough fractions with increasing total loaded amount [mgₚᵣₒₜᵢₑₙ/mL_{chromatography medium}] of hydrophilic mab2 in 400 mM Tris/Acetate buffer compared to 70 mM Tris/Acetate buffers containing the antichaotropic salts Na₂SO₄, KCl, (NH₄)₂SO₄, K₂SO₄ or NaCl in flowthrough mode on MMAEX Capto™ adhere ImpRes RCs for a load condition of pH 8 and a conductivity of 10 mS/cm.

FIG. 18B HMW removal value [%] of flowthrough fractions with increasing total loaded amount [mgₚᵣₒₜᵢₑₙ/mL_{chromatography medium}] of the hydrophobic mab7 in 400 mM Tris/Acetate buffer compared to 70 mM Tris/Acetate buffers containing the antichaotropic salts Na₂SO₄, KCl, (NH₄)₂SO₄, K₂SO₄ or NaCl in flowthrough mode on MMAEX Capto™ adhere ImpRes RCs for a load condition of pH 8 and a conductivity of 10 mS/cm.

FIG. 19 HMW value [%] of flowthrough fractions with increasing total loaded amount [gₚᵣₒₜᵢₑₙ/L_{chromatography medium}] of the hydrophilic mab2 in a load condition containing 70 mM Tris/Acetate, pH 8 and different molarities of the antichaotropic salt Na₂SO₄ on MMAEX Capto™ adhere ImpRes RCs.

FIG. 20 HMW value [%] of flowthrough fractions with increasing total loaded amount [gₚᵣₒₜᵢₑₙ/L_{chromatography medium}] of the hydrophilic mab2 in a load condition containing 70 mM Tris/Acetate, pH 8 and different molarities of the antichaotropic salt NaCl on MMAEX Capto™ adhere ImpRes RCs.

FIG. 21 HMW value [%] of flowthrough fractions with increasing total loaded amount [gₚᵣₒₜᵢₑₙ/L_{chromatography medium}] of the hydrophilic mab2 in a load condition containing 70 mM Tris/Acetate, pH 8 and different molarities of the antichaotropic salt (NH₄)₂SO₄ on MMAEX Capto™ adhere ImpRes RCs.

FIG. 22 HMW value [%] of flowthrough fractions with increasing total loaded amount [gₚᵣₒₜᵢₑₙ/L_{chromatography medium}] of the hydrophilic mab2 in a load condition containing 70 mM Tris/Acetate, pH 8 and different molarities of the antichaotropic salt KCl on MMAEX Capto™ adhere ImpRes RCs.

FIG. 23 HMW value [%] of flowthrough fractions with increasing total loaded amount [gₚᵣₒₜᵢₑₙ/L_{chromatography medium}] of the hydrophilic mab2 in a load condition containing 70 mM Tris/Acetate, pH 8 and different molarities of the antichaotropic salt K₂SO₄ on MMAEX Capto™ adhere ImpRes RCs.

FIG. 24A HMW removal value [%] of flowthrough samples of the hydrophilic mab2 in Tris/Acetate buffer containing the antichaotropic salt (NH₄)₂SO₄ with molarities up to 800 mM at pH 5.5 to pH 8.0 and a load capacity of 150 gₚᵣₒₜₑᵢₙ/L_{chromatography medium} using filterplate experiments with the MMAEX Capto™ adhere ImpRes.

FIG. 24B HMW removal value [%] of flowthrough samples of the hydrophilic mab4 in Tris/Acetate buffer containing the antichaotropic salt (NH₄)₂SO₄ with molarities up to 800 mM at pH 5.5 to pH 8.0 and a load capacity of 150 gₚᵣₒₜₑᵢₙ/L_{chromatography medium} using filterplate experiments with the MMAEX Capto™ adhere ImpRes.

FIG. 24C HMW removal value [%] of flowthrough samples of the hydrophobic mab6 in Tris/Acetate buffer containing the antichaotropic salt (NH₄)₂SO₄ with molarities up to 650 mM at pH 5.5 to pH 8.0 and a load capacity of 150 gₚᵣₒₜₑᵢₙ/L_{chromatography medium} using filterplate experiments with the MMAEX Capto™ adhere ImpRes.

FIG. 25A HMW removal value [%] of flowthrough samples of the hydrophilic mab2 in Tris/Acetate buffer containing the antichaotropic salt KCl with molarities up to 800 mM at pH 5.5 to pH 8.0 and a load capacity of 150 gₚᵣₒₜₑᵢₙ/L_{chromatography medium} using filterplate experiments with the MMAEX Capto™ adhere ImpRes.

FIG. 25B HMW removal value [%] of flowthrough samples of the hydrophilic mab4 in Tris/Acetate buffer containing the antichaotropic salt KCl with molarities up to 800 mM at pH 5.5 to pH 8.0 and a load capacity of 150 gₚᵣₒₜₑᵢₙ/L_{chromatography medium} using filterplate experiments with the MMAEX Capto™ adhere ImpRes.

FIG. 25C HMW removal value [%] of flowthrough samples of the hydrophobic mab6 in Tris/Acetate buffer containing the antichaotropic salt KCl with molarities up to 800 mM at pH 5.5 to pH 8.0 and a load capacity of 150 gₚᵣₒₜₑᵢₙ/L_{chromatography medium} using filterplate experiments with the MMAEX Capto™ adhere ImpRes.

FIG. 26A HMW removal value [%] of flowthrough samples of the hydrophilic mab2 in Tris/Acetate buffer containing the chaotropic salt Gua/HCl with molarities up to 800 mM at pH 5.5 to pH 8.0 and a load capacity of 150 gₚᵣₒₜₑᵢₙ/L_{chromatography medium} using filterplate experiments with the MMAEX Capto™ adhere ImpRes.

FIG. 26B HMW removal value [%] of flowthrough samples of the hydrophilic mab4 in Tris/Acetate buffer containing the chaotropic salt Gua/HCl with molarities up to 800 mM at pH 5.5 to pH 8.0 and a load capacity of 150 gₚᵣₒₜₑᵢₙ/L_{chromatography medium} using filterplate experiments with the MMAEX Capto™ adhere ImpRes.

FIG. 26C HMW removal value [%] of flowthrough samples of the hydrophobic mab6 in Tris/Acetate buffer containing the chaotropic salt Gua/HCl with molarities up to 800 mM at pH 5.5 to pH 8.0 and a load capacity of 150 gₚᵣₒₜₑᵢₙ/L_{chromatography medium} using filterplate experiments with the MMAEX Capto™ adhere ImpRes.

FIG. 27A HMW removal value [%] of flowthrough samples of the hydrophilic mab2 in Tris/Acetate buffer containing chaotropic salt urea with molarities up to 800 mM at pH 5.5 to pH 8.0 and a load capacity of 150 gₚᵣₒₜₑᵢₙ/L_{chromatography medium} using filterplate experiments with the MMAEX Capto™ adhere ImpRes.

FIG. 27B HMW removal value [%] of flowthrough samples of the hydrophilic mab4 in Tris/Acetate buffer containing the chaotropic salt urea with molarities up to 800 mM at pH 5.5 to pH 8.0 and a load capacity of 150 gₚᵣₒₜₑᵢₙ/L_{chromatography medium} using filterplate experiments with the MMAEX Capto™ adhere ImpRes.

FIG. 27C HMW removal value [%] of flowthrough samples of the hydrophobic mab6 in Tris/Acetate buffer containing the chaotropic salt urea with molarities up to 800 mM at pH 5.5 to pH 8.0 and a load capacity of 150 gₚᵣₒₜₑᵢₙ/L_{chromatography medium} using filterplate experiments with the MMAEX Capto™ adhere ImpRes.

FIG. 28A HMW removal value [%] of flowthrough samples of the hydrophilic mab2 in Tris/Acetate buffer containing the antichaotropic salt Na₂SO₄ with molarities up to 800 mM at pH 4.0 to pH 9.0 and a load capacity of 150 gₚᵣₒₜₑᵢₙ/L_{chromatography medium} using robotic filterplate experiments with the MMAEX Capto™ adhere ImpRes.

FIG. 28B HMW removal value [%] of flowthrough samples of the hydrophilic mab2 in Tris/Acetate buffer with increasing Tris molarities up to 1000 mM at pH 4.0 to pH 9.0 and a load capacity of 150 gₚᵣₒₜₑᵢₙ/L_{chromatography medium} using robotic filterplate experiments with the MMAEX Capto™ adhere ImpRes.

FIG. 29A HMW removal value [%] of flowthrough samples of the hydrophilic mab2 in Tris/Acetate buffer containing the antichaotropic salt (NH₄)₂SO₄ with molarities up to 800 mM at pH 5.5 to pH 8.0 and a load capacity of 150 gₚᵣₒₜₑᵢₙ/L_{chromatography medium} using filterplate experiments with the MMAEX Capto™ adhere ImpRes.

FIG. 29B HMW removal value [%] of flowthrough samples of the hydrophilic mab2 in Tris/Acetate buffer containing the antichaotropic salt (NH₄)₂SO₄ with molarities up to 800 mM at pH 5.5 to pH 8.0 and a load capacity of 150 gₚᵣₒₜₑᵢₙ/L_{chromatography medium} using filterplate experiments with the MMAEX Capto™ adhere.

FIG. 29C HMW removal value [%] of flowthrough samples of the hydrophilic mab2 in Tris/Acetate buffer containing the antichaotropic salt (NH₄)₂SO₄ with molarities up to 800 mM at pH 5.5 to pH 8.0 and a load capacity of 150 gₚᵣₒₜₑᵢₙ/L_{chromatography medium} using filterplate experiments with the MMAEX Nuvia aPrime.

FIG. 30A HMW removal value [%] of flowthrough samples of the hydrophilic mab2 in Tris/Acetate buffer containing the antichaotropic salt KCl with molarities up to 800 mM at pH 5.5 to pH 8.0 and a load capacity of 150 gₚᵣₒₜₑᵢₙ/L_{chromatography medium} using filterplate experiments with the MMAEX Capto™ adhere ImpRes.

FIG. 30B HMW removal value [%] of flowthrough samples of the hydrophilic mab2 in Tris/Acetate buffer containing the antichaotropic salt KCl with molarities up to 800 mM at pH 5.5 to pH 8.0 and a load capacity of 150 gₚᵣₒₜₑᵢₙ/L_{chromatography medium} using filterplate experiments with the MMAEX Capto™ adhere.

FIG. 30C HMW removal value [%] of flowthrough samples of the hydrophilic mab2 in Tris/Acetate buffer containing the antichaotropic salt KCl with molarities up to 800 mM at pH 5.5 to pH 8.0 and a load capacity of 150 gₚᵣₒₜₑᵢₙ/L_{chromatography medium} using filterplate experiments with the MMAEX Nuvia aPrime.

FIG. 31A HMW removal value [%] of flowthrough samples of the hydrophilic mab2 in Tris/Acetate buffer containing the chaotropic salt Gua/HCl with molarities up to 800 mM at pH 5.5 to pH 8.0 and a load capacity of 150 gₚᵣₒₜₑᵢₙ/L_{chromatography medium} using filterplate experiments with the MMAEX Capto™ adhere ImpRes.

FIG. 31B HMW removal value [%] of flowthrough samples of the hydrophilic mab2 in Tris/Acetate buffer containing the chaotropic salt Gua/HCl with molarities up to 800 mM at pH 5.5 to pH 8.0 and a load capacity of 150 gₚᵣₒₜₑᵢₙ/L_{chromatography medium} using filterplate experiments with the MMAEX Capto™ adhere.

FIG. 31C HMW removal value [%] of flowthrough samples of the hydrophilic mab2 in Tris/Acetate buffer containing the chaotropic salt Gua/HCl with molarities up to 800 mM at pH 5.5 to pH 8.0 and a load capacity of 150 gₚᵣₒₜₑᵢₙ/L_{chromatography medium} using filterplate experiments with the MMAEX Nuvia aPrime.

FIG. 32A HMW removal value [%] of flowthrough samples of the hydrophilic mab2 in Tris/Acetate buffer containing the chaotropic salt urea with molarities up to 800 mM at pH 5.5 to pH 8.0 and a load capacity of 150 gₚᵣₒₜₑᵢₙ/L_{chromatography medium} using filterplate experiments with the MMAEX Capto™ adhere ImpRes.

FIG. 32B HMW removal value [%] of flowthrough samples of the hydrophilic mab2 in Tris/Acetate buffer containing the chaotropic salt urea with molarities up to 800 mM at pH 5.5 to pH 8.0 and a load capacity of 150 gₚᵣₒₜₑᵢₙ/L_{chromatography medium} using filterplate experiments with the MMAEX Capto™ adhere.

FIG. 32C HMW removal value [%] of flowthrough samples of the hydrophilic mab2 in Tris/Acetate buffer containing the chaotropic salt urea with molarities up to 800 mM at pH 5.5 to pH 8.0 and a load capacity of 150 gₚᵣₒₜₑᵢₙ/L_{chromatography medium} using filterplate experiments with the MMAEX Nuvia aPrime.

FIG. 33A HMW removal value [%] of flowthrough samples of the hydrophilic mab2 in Tris/Acetate buffer containing the antichaotropic salt (NH₄)₂SO₄ with molarities up to 800 mM at pH 5.5 to pH 8.0 and a load capacity of 150 gₚᵣₒₜₑᵢₙ/L_{chromatography medium} using filterplate experiments with the MMAEX Capto™ adhere ImpRes.

FIG. 33B HMW removal value [%] of flowthrough samples of the hydrophobic mab6 in Tris/Acetate buffer containing the antichaotropic salt (NH₄)₂SO₄ with molarities up to 650 mM at pH 5.5 to pH 8.0 and a load capacity of 150 gₚᵣₒₜₑᵢₙ/L_{chromatography medium} using filterplate experiments with the MMAEX Capto™ adhere ImpRes.

FIG. 34A HMW removal value [%] of flowthrough samples of the hydrophilic mab2 in Tris/Acetate buffer containing the antichaotropic salt Na₂SO₄ with molarities up to 800 mM at pH 4.0 to pH 9.0 and a load capacity of 150 gₚᵣₒₜₑᵢₙ/L_{chromatography medium} using filterplate experiments with the AEX chromatography medium Q Sepharose FF.

FIG. 34B HMW removal value [%] of flowthrough samples of the hydrophobic mab6 in Tris/Acetate buffer containing the antichaotropic salt Na₂SO₄ with molarities up to 450 mM at pH 4.0 to pH 9.0 and a load capacity of 150 gₚᵣₒₜₑᵢₙ/L_{chromatography medium} using filterplate experiments with the AEX chromatography medium Q Sepharose FF.

FIG. 35A HMW removal value [%] of flowthrough samples of the hydrophilic mab2 in Tris/Acetate buffer containing the antichaotropic salt Na₂SO₄ with molarities up to 650 mM at pH 4.0 to pH 9.0 and a load capacity of 150 gₚᵣₒₜₑᵢₙ/L_{chromatography medium} using filterplate experiments with the HIC chromatography medium Phenyl Sepharose 6 FF.

FIG. 35B HMW removal value [%] of flowthrough samples of the hydrophobic mab6 in Tris/Acetate buffer containing the antichaotropic salt Na₂SO₄ with molarities up to 650 mM at pH 4.0 to pH 9.0 and a load capacity of 150 gₚᵣₒₜₑᵢₙ/L_{chromatography medium} using filterplate experiments with the HIC chromatography medium Phenyl Sepharose 6 FF.

FIG. 36A HMW removal value [%] of flowthrough samples of the hydrophilic mab2 in Tris/Acetate buffer containing the antichaotropic salt Na₂SO₄ with molarities up to 800 mM at pH 4.0 to pH 9.0 and a load capacity of 75 gₚᵣₒₜₑᵢₙ/L_{chromatography medium} using filterplate experiments with the MMCEX Capto™ MMC ImpRes.

FIG. 36B HMW removal value [%] of flowthrough samples of the hydrophobic mab6 in Tris/Acetate buffer containing the antichaotropic salt Na₂SO₄ with molarities up to 650 mM at pH 4.0 to pH 9.0 and a load capacity of 75 gₚᵣₒₜₑᵢₙ/L_{chromatography medium} using filterplate experiments with the MMCEX Capto™ MMC ImpRes.

FIG. 37 Mainpeak value [%] of flowthrough fractions with increasing total loaded amount [gₚᵣₒₜₑᵢₙ/L_{chromatography medium}] of the hydrophilic mab2 with two loads at pH 8 and a conductivity of 9 mS/cm containing different molarities of Na₂SO₄ (20 mM compared to 40 mM Na₂SO₄) on lab scale MMAEX Capto™ adhere ImpRes columns.

FIG. 38 Mainpeak value [%] of flowthrough fractions with increasing total loaded amount [gₚᵣₒₜᵢₑₙ/L_{chromatography medium}] of the hydrophilic mab2 in a load condition containing 70 mM Tris/Acetate, pH 8 and different molarities of the antichaotropic salt Na₂SO₄ on lab scale Capto™ adhere ImpRes columns.

FIG. 39 Mainpeak value [%] of flowthrough fractions with increasing total loaded amount [gₚᵣₒₜₑᵢₙ/L_{chromatography medium}] of the hydrophilic mab2 in a load condition containing 70 mM Tris/Acetate, pH 7 and different molarities of the antichaotropic salt Na₂SO₄ on lab scale MMAEX Capto™ adhere ImpRes columns.

FIG. 40 Mainpeak value of pools calculated using the average mainpeak value of the fractions of the hydrophilic mab2 at a loaded amount of 150 gₚᵣₒₜₑᵢₙ/L_{chromatography medium} in a load condition containing 70 mM Tris/Acetate, pH 8 and different molarities of the antichaotropic salt Na₂SO₄ on lab scale MMAEX Capto™ adhere ImpRes columns.

FIG. 41A RNA log reduction of flowthrough samples of the hydrophilic mab1 in Tris/Acetate buffer containing the antichaotropic salt sodium sulfate with molarities up to 400 mM at pH 5.0 to pH 8.0 and a load capacity of 150 gₚᵣₒₜₑᵢₙ/L_{chromatography medium} using filter plate experiments with the MMAEX Capto™ adhere ImpRes.

FIG. 41B RNA log reduction of flowthrough samples of the hydrophilic mab2 in Tris/Acetate buffer containing the antichaotropic salt sodium sulfate with molarities up to 400 mM at pH 5.0 to pH 8.0 and a load capacity of 150 gₚᵣₒₜₑᵢₙ/L_{chromatography medium} using filter plate experiments with the MMAEX Capto™ adhere ImpRes.

FIG. 41C RNA log reduction of flowthrough samples of the hydrophobic mab7 in Tris/Acetate buffer containing the antichaotropic salt sodium sulfate with molarities up to 400 mM at pH 5.0 to pH 8.0 and a load capacity of 150 gₚᵣₒₜₑᵢₙ/L_{chromatography medium} using filter plate experiments with the MMAEX Capto™ adhere ImpRes.

FIG. 41D RNA log reduction of flowthrough samples of the hydrophobic mab9 in Tris/Acetate buffer containing the antichaotropic salt sodium sulfate with molarities up to 400 mM at pH 5.0 to pH 8.0 and a load capacity of 150 gₚᵣₒₜₑᵢₙ/L_{chromatography medium} using filter plate experiments with the MMAEX Capto™ adhere ImpRes.

FIG. 42A RNA log reduction of flowthrough samples of the hydrophilic mab1 in Tris/Acetate buffer containing the antichaotropic salt sodium sulfate with conductivities up to 34 mS/cm at pH 5.0 to pH 8.0 and a load capacity of 150 gₚᵣₒₜₑᵢₙ/L_{chromatography medium} using filter plate experiments with the MMAEX Capto™ adhere ImpRes.

FIG. 42B RNA log reduction of flowthrough samples of the hydrophilic mab2 in Tris/Acetate buffer containing the antichaotropic salt sodium sulfate with conductivities up to 34 mS/cm at pH 5.0 to pH 8.0 and a load capacity of 150 gₚᵣₒₜₑᵢₙ/L_{chromatography medium} using filter plate experiments with the MMAEX Capto™ adhere ImpRes.

FIG. 42C RNA log reduction of flowthrough samples of the hydrophobic mab7 in Tris/Acetate buffer containing the antichaotropic salt sodium sulfate with conductivities up to 34 mS/cm at pH 5.0 to pH 8.0 and a load capacity of 150 gₚᵣₒₜₑᵢₙ/L_{chromatography medium} using filter plate experiments with the MMAEX Capto™ adhere ImpRes.

FIG. 42D RNA log reduction of flowthrough samples of the hydrophobic mab9 in Tris/Acetate buffer containing the antichaotropic salt sodium sulfate with conductivities up to 34 mS/cm at pH 5.0 to pH 8.0 and a load capacity of 150 gₚᵣₒₜₑᵢₙ/L_{chromatography medium} using filter plate experiments with the MMAEX Capto™ adhere ImpRes.

FIG. 43A RNA log reduction of flowthrough samples of the hydrophilic mab1 in Tris/Acetate buffer with increasing Tris molarities up to 1100 mM at pH 5.0 to pH 8.0 and a load capacity of 150 gₚᵣₒₜₑᵢₙ/L_{chromatography medium} using filter plate experiments with the MMAEX Capto™ adhere ImpRes.

FIG. 43B RNA log reduction of flowthrough samples of the hydrophilic mab2 in Tris/Acetate buffer with increasing Tris molarities up to 1100 mM at pH 5.0 to pH 8.0 and a load capacity of 150 gₚᵣₒₜₑᵢₙ/L_{chromatography medium} using filter plate experiments with the MMAEX Capto™ adhere ImpRes.

FIG. 43C RNA log reduction of flowthrough samples of the hydrophobic mab7 in Tris/Acetate buffer with increasing Tris molarities up to 1100 mM at pH 5.0 to pH 8.0 and a load capacity of 150 gₚᵣₒₜₑᵢₙ/L_{chromatography medium} using filter plate experiments with the MMAEX Capto™ adhere ImpRes.

FIG. 43D RNA log reduction of flowthrough samples of the hydrophobic mab9 in Tris/Acetatebuffer with increasing Tris molarities up to 1100 mM at pH 5.0 to pH 8.0 and a load capacity of 150 gₚᵣₒₜₑᵢₙ/L_{chromatography medium} using filter plate experiments with the MMAEX Capto™ adhere ImpRes.

FIG. 44A RNA log reduction of flowthrough samples of the hydrophilic mab1 in Tris/Acetate buffer with increasing conductivities up to 19 mS/cm at pH 5.0 to pH 8.0 and a load capacity of 150 gₚᵣₒₜₑᵢₙ/L_{chromatography medium} using filter plate experiments with the MMAEX Capto™ adhere ImpRes.

FIG. 44B RNA log reduction of flowthrough samples of the hydrophilic mab2 in Tris/Acetate buffer with increasing conductivities up to 19 mS/cm at pH 5.0 to pH 8.0 and a load capacity of 150 gₚᵣₒₜₑᵢₙ/L_{chromatography medium} using filter plate experiments with the MMAEX Capto™ adhere ImpRes.

FIG. 44C RNA log reduction of flowthrough samples of the hydrophobic mab7 in Tris/Acetate buffer with increasing conductivities up to 19 mS/cm at pH 5.0 to pH 8.0 and a load capacity of 150 gₚᵣₒₜₑᵢₙ/L_{chromatography medium} using filter plate experiments with the MMAEX Capto™ adhere ImpRes.

FIG. 44D RNA log reduction of flowthrough samples of the hydrophobic mab9 in Tris/Acetatebuffer with increasing conductivities up to 19 mS/cm at pH 5.0 to pH 8.0 and a load capacity of 150 gₚᵣₒₜₑᵢₙ/L_{chromatography medium} using filter plate experiments with the MMAEX Capto™ adhere ImpRes.

### EXPERIMENTAL PART

### Material & Methods

### 1. Proteins

The molecules used herein were humanized IgG1 monoclonal antibodies (mabs) produced in Chinese hamster ovary cells. Starting material used to load the mixed mode chromatography columns was an affinity chromatography column eluate (denoted as “affinity column pool”). The molecules encompassed standard IgG-like mabs and complex antibody formats, e.g. bispecific CrossMab format, mabs containing a bound ligand (2+1 C format) and T-cell binding mabs (2+1 N format; TCB). The pI of the molecules was in the range of 8.0-9.4.

For the RVLP removal studies starting material used to load the mixed mode chromatography columns was a second column chromatography eluate (i.e. an eluate after an affinity chromatography and a subsequent second chromatography run; denoted as “second column pool”). The second chromatography run could for example be perfomed on a cation exchange chromatography material (as is the case for e.g. mab9), an anion exchange chromatography material or a mixed mode chromatography material such as a mixed mode anion exchange chromatography material (as is the case for e.g. mab1, mab2 or mab7).

The method for determination of the retention times is described below in Materials and Methods item 10. The retention times of the mabs determined with this method were in the range from 19 min. to 41 min. An overview of the retention times of the mabs is given in Table. The retention time of Rituximab was found to be the cut-point for defining hydrophilic and hydrophobic mabs. Mabs with a retention time≤retention time_{rituximab}, i.e. that have the same or a shorter retention time as rituximab, are defined to be hydrophilic, mabs with retention time>retention time_{rituximab}, i.e. have a longer retention time, are defined to be hydrophobic.

TABLE MM-1
Mabs and retention times

| | | retention |
|---|---|---|
| | denoted | time |
| mab | as | [min] |
| bivalent, monospecific full-length IgG1 antibody | mab1 | 19.4 |
| specifically binding to antigen 1 | | |
| bivalent, monospecific full-length IgG1 antibody | mab2 | 21.7 |
| specifically binding to antigen 2 | | |
| antibody in 2 + 1 N format specifically binding to | mab4 | 29.5 |
| antigens 3 and 4 | | |
| bivalent, monospecific full-length IgG1 antibody | mab5 | 29.6 |
| specifically binding to antigen 5 | | |
| Rituximab | Cut-point | 32.0 |
| bivalent, bispecific full-length IgG1 antibody in | mab6 | 35.8 |
| CrossMab format specifically binding to antigens | | |
| 6 and 7, variant 1 | | |
| bivalent, bispecific full-length IgG1 antibody in | mab7 | 35.8 |
| CrossMab format specifically binding to antigens | | |
| 6 and 7, variant 2 | | |
| antibody in 2 + 1 C format specifically binding to | mab8 | 41.2 |
| antigens 8 and 9 | | |
| antibody in 2 + 1 N format specifically binding to | mab9 | 41.2 |
| antigens 4 and 8 | | |

### 2. Chemicals

K₂HPO₄, KH₂PO₄, KCl, Na₂HPO₄×₂H₂O, NaH₂PO₄×H₂O, ethanol, Tris(hydroxymethyl)-aminomethan, acetic acid, citric acid, sodium sulfate, ammonium sulfate, sodium chloride, potassium chloride, potassium sulfate, guanidinium/hydrochloride, urea, NaOH were purchased from the manufacturers as listed below and used as provided by the manufacturer.

Merck KGaA: K₂HPO₄, KH₂PO₄, KCl, Na₂HPO_{4×2}H₂O, NaH₂PO₄×H₂O, ethanol, acetic acid, citric acid, sodium sulfate, ammonium sulfate, sodium chloride, potassium chloride, guanidinium chloride, urea, NaOH
ANGUS Chemie GmbH: Tris(hydroxymethyl)-aminomethan
Sigma Aldrich and Merck KGaA: ammonium sulfate
Thermo Fisher Scientific GmbH: potassium sulfate

### 3. Robocolumns (RCs)

Robocolumns™ (RC) were purchased from Repligen GmbH (Ravensburg, Germany):
Capto™ adhere ImpRes; PN 01100408R; 200 μL

### 4. Chromatography Resins

The following chromatography resins were used herein:
Capto™ adhere ImpRes (Cytiva (Formerly GE Healthcare), Uppsala, Sweden)
Capto™ adhere (Cytiva (Formerly GE Healthcare))
Nuvia aPrime4A (Bio-Rad Laboratories, Inc., USA)
Q Sepharose FF (Cytiva (Formerly GE Healthcare))
Phenyl Sepharose 6 FF (high sub) (Cytiva (Formerly GE Healthcare))
Capto™ MMC ImpRes (Cytiva (Formerly GE Healthcare))

### 5. Robotic Labware

Filter plates: PALL; AcroPrep Advance 96 Well, 1 mL, 0.45 μm, REF 8184
MTP UV Plates: UV Microtiter Plates, Thermo Scientific

### 6. Materials for Load Preparation

Centrifuge: Heraeus Multifige 3 S-R; Rotor: 75006445
Amicon Ultra Centrifugal Filters (Merck Millipore, Ultracel-30K)
Slide-A-Lyzer Dialysis Cassettes (Thermo Scientific, 20 000-30 000 MWCO)
Minisart Syringe Filter (Sartorius Minisart High Flow, 0.22 μm)

### 7. Robotic Systems

### 7.1. Tecan Freedom EVO 150

A Tecan Freedom EVO 150 (Tecan Deutschland GmbH, Crailsheim, Germany) liquid handling system (LHS) was used to perform the RC runs. The EVO 150 was equipped with one liquid handling arm (LiHa) and one excentric gripper, an atoll bridge for the RCs and an Infinite M200 NanoQuant plate reader (Tecan Deutschland GmbH, Crailsheim, Germany). The LHS was controlled by the software Freedom EVOware (Tecan Deutschland GmbH, Crailsheim, Germany). The software used to control the plate reader was Magellan (Tecan Deutschland GmbH, Crailsheim, Germany). The platform was additionally equipped with a Te-Stack™ for storage of 96 well collection plates (microtiter plates) and a Te-Slide™ for plate transport and fraction collection. The LiHa was capable of processing volumes of 10 μL to 1000 μL and was equipped with 1000 μL dilutor syringes. The LiHa consisted of eight separately controllable channels equipped with eight fixed stainless steel needles. All RC runs were performed with Capto™ adhere ImpRes resin. The column dimensions were 1 cm length×0.5 cm diameter and a bed volume of 200 μL.

### 7.2. Hamilton Microlab STARlet

A Hamilton Microlab STARlet roboter was used for the preparation of the filter plate used herein. The roboter was equipped with eight 1000 μL pipetting channels and a shaker. A 50% slurry of resin in water (v/v) was produced and placed in a glass vial on the shaker. The LiHa was equipped with wide bore 1000 μL tips (cut) to transfer the resin from the shaker to the filter plate. A filter plate containing 50 μL resin per well was produced. The storage solution was water.

### 7.3. Tecan Freedom EVO 200

A Tecan Freedom EVO 200 (Tecan Deutschland GmbH, Crailsheim, Germany) liquid handling system was used to prepare the load plate and buffer plate as used herein and to perform the runs. The Tecan Freedom EVO 200 was equipped with one liquid handling arm (LiHa), one excentric gripper, a Te-Shake™, a Te-Stack™ for storage of microplates, a Te-Slide™ for plate transport and an Infinite M200 plate reader (Tecan Deutschalnd GmbH, Crailsheim, Germany). Additionally, a centrifuge (Rotanta 46RSC, Hettich, Germany) was integrated into the worktable to remove the supernatant after incubation. The LiHa was capable of processing volumes of 10 μL to 1000 μL and was equipped with 1000 μL dilutor syringes and consisted of eight separately controllable channels equipped with eight fixed stainless steel pipette tips. The Tecan robot was controlled by the software Evoware. The software used to control the plate reader was Magellan.

### 8. Determination of Protein Purity

Protein purity in terms of monomer content and high molecular weights species was determined by size-exclusion High Performance Liquid Chromatography (SE-HPLC) using a HPLC system (Thermo Fisher). Protein separation was performed on a TSK-Gel G3000SWXL (7.8×300 mm; 5 μm) column (TOSOH Bioscience, P/N 08541) with a flow rate of 0.5-1.0 ml/min using 0.2 M K₂HPO₄/KH₂PO₄, 0.25 M KCl, pH 7.0 as eluent.

The following conditions were used:
wavelength: 280 nm
isocratic; 30 min
column temperature: 25° C.
sample temperature: 10° C.
applied quantity: 150 g protein

### 9. Determination of Protein Concentration

### 9.1. Determination Using Cuvette

Protein concentrations were determined by UV spectroscopy using a Spectramax Plus (Molecular Devices, Munich, Germany). The measurement was executed in a cuvette. Protein samples were diluted in water. Concentrations were determined according to the following Equation 1 deriving from Lambert-Beer law:

$$( {A}_{280} - {A}_{320} ) = ε \cdot c \cdot d \qquad ( 1 )$$

A absorbance, c protein concentration [mg/ml], ε extinction coefficient [ml/(mg*cm)], d path length [cm].

### 9.2. Determination Using Microplate

Protein concentrations were determined by UV spectroscopy using an Infinite plate reader (Tecan Deutschland GmbH, Crailsheim, Germany). The measurement was executed in a microplate. Protein samples were diluted in water. Concentrations were determined according to Equation (1).

The path length d was calculated with the following Equation 2:

$$pathlength d = \frac{( {A}_{998 nm} - {A}_{900 nm} )}{{A}_{water} {cm}^{- 1}} \qquad ( 2 )$$

A_{water}=0.159 OD/cm (corresponding to application note TECAN; Doc No. N129013 02).

### 10. Determination of Retention Time and Hydrophobicity

### 10.1. Equipment

HPLC device with integrated data collection system; Dionex (now Thermo Fisher Scientific)
0.2 μm membrane filters; e.g. Pall Life Sciences Supor®-200, Catalog no. 60301
Tosoh Bioscience, TSKgel® Ether-5 PW HPLC Column, 10 μm, 7.5 mm×75 mm, Catalog no. 0008641, i.e. a hydrophobic interaction chromatography column with an inner diameter of 2 mm, a column length of 75 mm and a particle size of 10 μm. The column has a polymethacrylate base material (matrix) with polyether groups as ligand (ethyl ether groups).

### 10.2. Working Solutions:

Eluent A: 25 mM Na-phosphate buffer comprising 1.5 M (NH₄)₂SO₄ adjusted to pH 7.0
Eluent B: 25 mM Na-phosphate buffer, adjusted to pH 7.0

### 10.3. Hydrophobic Interaction Chromatography (HIC) Standard

For standard preparation Rituximab is diluted to 1 mg/ml with a suitable dilution buffer. 20 μl (=20 μg) of the HIC standard are injected. The peak of Rituximab will appear close to 32 min run time, i.e. has a retention time close to 32 min.

### 10.4. Samples

Samples are diluted to a concentration of 1 mg/ml. If samples are already at a concentration of 1 mg/ml, no dilution is necessary. 20 μl (=20 μg) of the 1 mg/ml solution are injected.

### 10.5. Blank

20 μl of the dilution buffer were injected.

### 10.6. Conditioning of New Columns

Columns were provided in ultrapure water. Before the first run a new column was prepared as follows:

1) the column was transferred at ambient temperature to Eluent B: the flow was slowly increased from 0.0 ml/min to 0.8 ml/min within a minimum of 40 min; the column was washed thereafter with Eluent B at 0.8 ml/min until a stable baseline was reached (usually after 60 min).

2) Gradient run: a linear gradient from 0% Eluent A to 100% Eluent A within 20 min. was run; the column was washed thereafter with Eluent A until a stable baseline was reached (usually after 60 min).

3) Saturation: the available standard was injected and processed according to 10.8. multiple times until three successive chromatograms were identical with regard to peak form, height and area; the column was thereafter used.

### 10.7. Conditioning of Used Columns

After mounting, column was washed at ambient temperature with ultrapure water. The flow was slowly increased from 0.0 ml/min to 0.8 ml/min within a minimum of 40 min. Thereafter the column was washed with Eluent A at 0.8 ml/min until a stable baseline was reached. The column was thereafter used.

### 10.8. Working Conditions and Sequence Layout

Before any sequence 1× Eluent A was injected, followed by an injection of 20 μl of 0.1 M NaOH and another injection of Eluent A.

The following working conditions were used:
Flow rate: 0.8 ml/min
Gradient:

| Time [min] | Eluent A [%] | Eluent B [%] |
|---|---|---|
| 0.0 | 100 | 0 |
| 2.0 | 100 | 0 |
| 3.0 | 87 | 13 |
| 5.0 | 87 | 13 |
| 57.0 | 0 | 100 |
| 62.0 | 0 | 100 |
| 63.0 | 100 | 0 |
| 65.0 | 100 | 0 |

Maximum pressure: 22 bar
Wavelength: 214 nm (in addition record 220 nm and 280 nm)
Injected protein: 20 μg
Column temperature: 40° C.±2° C.
Temperature in autosampler: 10° C.±4° C.

HIC standard and samples were measured in the following sequence:
1. Eluent A
2. 20 μl 0.1 M NaOH
3. Eluent A
4. HIC/reference
5. Blank (dilution buffer)
6. Samples 1 to n
7+n Blank (dilution buffer)

The retention times were determined at peak maximum.

Mabs eluting simultaneously with or prior to Rituximab (with shorter retention time) have found to be hydrophilic (retention time_{mab}≤retention time_{rituximab}) whereas mabs eluting after Rituximab (with longer retention time) have found to be hydrophobic (retention time_{mab}>retention time_{rituximab}).

### 11. Determination of RNA Concentration

In order to determine the removal of RVLPs, the RNA concentrations in the samples are determined as representative measurements.

Automated RNA-analytics are performed via the FLOW PCR setup system (Roche Diagnostics Gmbh). The system consists of 3 modules: FLOW PCR SETUP instrument (Roche Diagnostics GmbH, order no. 07101996001), MagNA Pure 96 instrument (Roche Diagnostics GmbH, order no. 06541089001) and LightCycler®480 instrument (Roche Diagnostics GmbH, order no. 05015278001). The determination of the RNA content is performed according to the manufacture's operating manuals. In brief, the RNA is isolated using the MagNA Pure 96 instrument. After a treatment with DNAse, the probes are measured in the LightCycler®480 instrument to quantify the RNA by means of the PCR technology (quantitative RT PCR). The RNA is therefore first converted to cDNA by reverse transcription (RT). Then, the cDNA is amplified in a PCR reaction and the concentration is quantified by way of comparison to a standard curve. Subsequently the result is converted to RNA content.

### Overview of the Examples

The examples section can be subdivided into four parts:

### Part I: Impact of Mab Hydrophobicity on High Molecular Weight (HMW) Impurity Reduction at Constant Conductivity

Flowthrough (FT) runs were performed with Capto™ adhere ImpRes RCs (Repligen) on a robotic system (Tecan Freedom 150). Up to 5 antichaotropic (ac) salts were chosen (Na₂SO₄, NaCl, (NH₄)₂SO₄, KCl, K₂SO₄). The FT of 7 mabs was collected and purity was analyzed by SE-HPLC. The HMW reduction achieved by adding an antichaotropic salt to the load were compared with the HMW reduction determined with a Tris/Acetate buffer, pH 8 and accordingly a Tris/Citrate buffer, pH 6 at same conductivity without containing an antichaotropic salt. It was shown that only for hydrophilic mabs (retention time_{mab}≤retention time_{rituximab}) an improved HMW reduction was achieved by addition of an antichaotropic salt to the load material compared to a load material having same conductivity under absence of an antichaotropic salt. In contrast to that, for hydrophobic mabs (retention time_{mab}>retention time_{rituximab}) no positive impact was observed with addition of an antichaotropic salt.

### Table MM-2

Table MM-2: summarizes the examples of part I.

TABLE MM-2
Part I-examples

| example | | | conductivity | |
|---|---|---|---|---|
| number | mabs | pH | [mS/cm] | salts used |
| Example 1 | mab 1, 2, | 8.0 | 20 | Na₂SO₄, NaCl, (NH₄)₂SO₄, |
| | 4-8 | | | KCl, K₂SO₄ |
| Example 2 | mab 1, 2, | 6.0 | 20 | Na₂SO₄, KCl |
| | 4-8 | | | |
| Example 3 | mab2 & | 8.0 & | 10 | Na₂SO₄, NaCl, (NH₄)₂SO₄, |
| | mab7 | 6.0 | | KCl, K₂SO₄ |

### Part II: Impact of Antichaotropic Salt Molarity on HMW Removal

In the 2^{nd} experimental part RC experiments and Kp (partition coefficient) screens were performed to investigate the impact of different molarities of antichaotropic salts on HMW reduction. In Example 4 a hydrophilic mab was loaded on Capto™ adhere ImpRes RCs with a molarity range up to 500 mM. Different salts, Na₂SO₄, NaCl, (NH₄)₂SO₄, KCl and K₂SO₄ were investigated at pH 8.0. It was shown that an increase in salt molarity could improve reduction of HMWs in the FT fractions.

In addition to these RC runs, Kp screens were run to show the impact of salt molarity on HMW reduction for a broader range of pH and molarity using Capto™ adhere ImpRes resin/chromatography material (Examples 5 and 6). For Example 5 three mabs were chosen, two hydrophilic mabs and one hydrophobic mab. For the Kp screens the investigated pH range was pH 5.5-8.0 and the molarity range 10-800 mM. The Kp screen HMW reduction confirmed the RC data. It was shown that for hydrophilic mabs an increasing salt molarity resulted in an improved HMW reduction whereas HMW reduction for the hydrophobic mab was not improved by increasing the antichaotropic salt molarity. To emphasize the need of an antichaotropic salt, chaotropic salts were investigated additionally. The Contour plots using the chaotropic salts did not show an improved HMW reduction with increasing salt molarity.

In Example 6 Kp screens were performed with mab2 and a screening range of pH 4-9 and molarities up to ˜900 mM. Within these Kp screens two sets of buffers were compared: one buffer containing the antichaotropic salt Na₂SO₄ and one buffer without an antichaotropic salt (only Tris/Acetate buffer).

Table MM-3 summarizes the examples of part II.

TABLE MM-3
Part II - examples

| | | | | salt | |
|---|---|---|---|---|---|
| example | | | | molarity | |
| number | procedure | mab | pH | [mM] | salts used |
| Example 4 | robocolumn | mab2 | 8.0 | 0-500 | Na₂SO₄, NaCl, |
| | | | | | (NH₄)₂SO₄, KCl, |
| | | | | | K₂SO₄ |
| Example 5 | Kp screen | mab2; | 5.5-8.0 | 10-800 | antichaotropic: |
| | | mab4; | | | (NH₄)₂SO₄, KCl |
| | | mab7 | | | chaotropic: Urea, |
| | | | | | Gua/HCl |
| Example 6 | Kp screen | mab2 | 4.0-9.0 | 10-~900 | Na₂SO₄, |
| | | | | | Tris/Acetate |

Part III: Comparison of HMW Removal with Other Resins

The 3^{rd} part consists of two examples: In example 7 HMW removals of a hydrophilic mab in a pH range of 5.5-8.0 and salt molarities of 10-800 mM were investigated. In this example the following mixed mode anion exchange (MMAEX) resins were compared: Capto™ adhere ImpRes, Capto™ adhere and Nuvia aPrime. All three resins showed an improved HMW reduction when an antichaotropic salt was added to the load solution comprising a hydrophilic mab.

Example 8 summarizes Kp screens for a MMAEX, an Anion exchange (AEX) resin, a HIC resin and a mixed mode cation exchange (MMCEX) resin with one hydrophilic and one hydrophobic mab. The investigated pH range was pH 4.0-9.0 and 5-850 mM salt. The flowthrough samples of the hydrophilic mab indicated an improved HMW reduction with increasing salt molarity for both ionic mixed mode resins (MMAEX and MMCEX). In contrast to that for the hydrophobic mab HMW reduction on the MMAEX resin was constant over the investigated range of salt molarity. For the MMCEX resin HMW reduction for the hydrophobic mab was not improved by increasing salt molarity below 500 mM Na₂SO₄. With the single mode resins Q Sepharose FF (AEX) no positive effect on HMW reduction was observed neither for the hydrophilic nor for the hydrophobic mab. For the Phenyl Sepharose 6 FF (high sub) (HIC) a positive impact of increasing salt molarity on HMW reduction was measured for the hydrophilic and hydrophobic mab.

Table MM-4 illustrates the examples for part III.

TABLE MM-4
Part III - examples

| | | | | salt | |
|---|---|---|---|---|---|
| example | | | | molarity | |
| number | resins | mab | pH | [mM] | Salts used |
| Example 7 | Capto adhere | mab2 | 5.5-8.0 | 10-800 | antichaotropic: |
| | ImpRes, | | | | (NH₄)₂SO₄, |
| | Capto adhere, | | | | KCl chaotropic: |
| | Nuvia aPrime | | | | Urea, Gua/HCl |
| Example 8 | Q Sepharose | mab2 | 4.0-9.0 | 5-850 | (NH₄)₂SO₄, |
| | FF, Phenyl | & | | | Na₂SO4 |
| | Sepharose | mab6 | | | |
| | 6FF | | | | |
| | (high sub), | | | | |
| | Capto MMC | | | | |
| | ImpRes | | | | |

### Part IV: Upscaling ÄKTA Column Runs

For the 4th part scale-up runs on an ÄKTA system were performed with Na₂SO₄ and mab2. Here an upscaling from Kp screen resin volumes of 50 μL and RC volumes of 200 μL to a column volume of 6.8 mL was done with Capto™ adhere ImpRes resin. Example 9 shows the Mainpeak value of the FT fractions for two runs with the same conductivity but different Na₂SO₄ molarities. Although the conductivity of both loads was equal, the HMW reduction for the load containing the higher molarity of Na₂SO₄ was significantly better. In Example 10 runs at pH 7 and pH 8 with different Na₂SO₄ molarities (and different conductivities) were described. An improved HMW reduction was determined with increasing salt molarity. These lab scale results confirmed the results achieved with the robotic systems in part II and III.

Table MM-5 illustrates the examples for part IV.

TABLE MM-5
Part IV - examples

| example | | | | conductivity | |
|---|---|---|---|---|---|
| number | resins | mab | pH | [mS/cm] | salts used |
| Example 9 | Capto | mab2 | 8 | 9 (different | Na₂SO₄ |
| | adhere | | | molarities: | |
| | ImpRes | | | 20 & 40 mM) | |
| Example 10 | Capto | mab2 | 7 | 5-12 | Na₂SO₄ |
| | adhere | | 8 | (0-60 mM) | |
| | ImpRes | | | | |

### Part V: Removal of RVLPs (Retrovirus Like Particles)

In addition to the removal of product-related impurities (like HMWs) it was also investigated whether the use of an antichaotropic salt in the purification of antibodies on a MM chromatography resin does also have an effect of the removal of retrovirus like particles (RVLPs).

In this respect two KpScreens were performed (Example 11 and Example 12).

### KpScreen: Impact of Antichaotropic Salt and Mab Hydrophobicity on RVLP Removal

Kp (partition coefficient) screens were performed to investigate the impact of an antichaotropic salt and mab hydrophobicity on RVLP reduction. The concentration of (RNA-containing) RVLPs was measured via the quantification of the concentration of RNA by means of quantitative RT PCR. Within these Kp screens two sets of buffers were compared: one buffer containing the antichaotropic salt Na₂SO₄ (Example 11) and one buffer without an antichaotropic salt (only Tris/Acetate buffer) (Example 12). Additionally four mabs were chosen, two hydrophilic mabs (mab1 and mab2) and two hydrophobic mabs (mab7 and mab9). The chromatography resin was Capto™ adhere ImpRes.

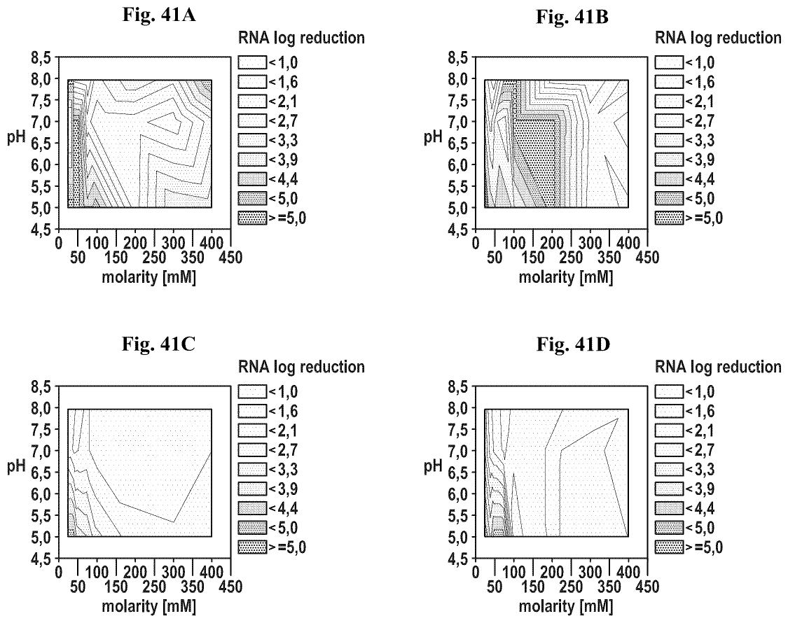

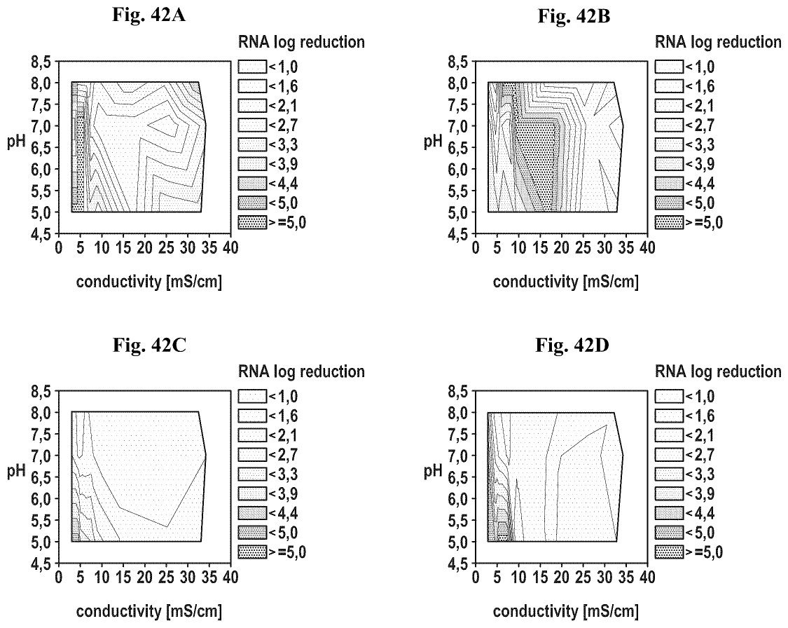

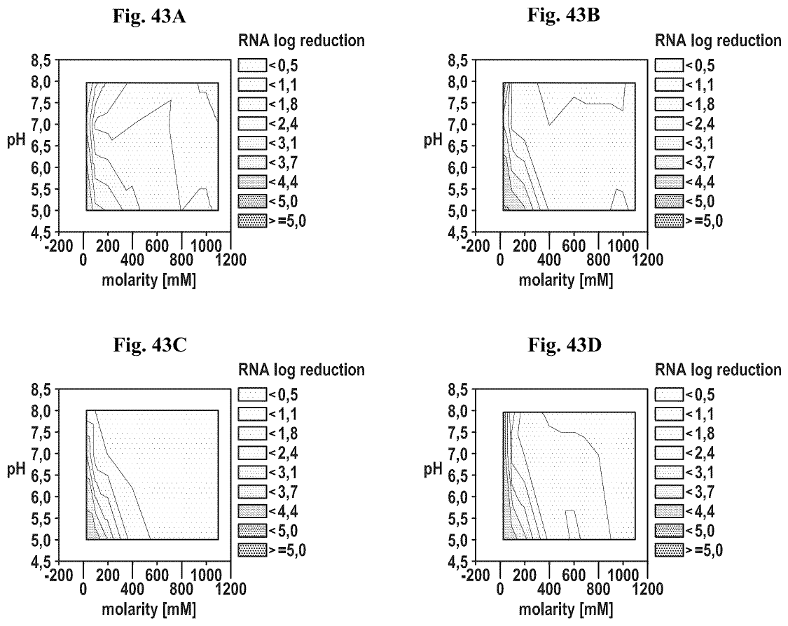

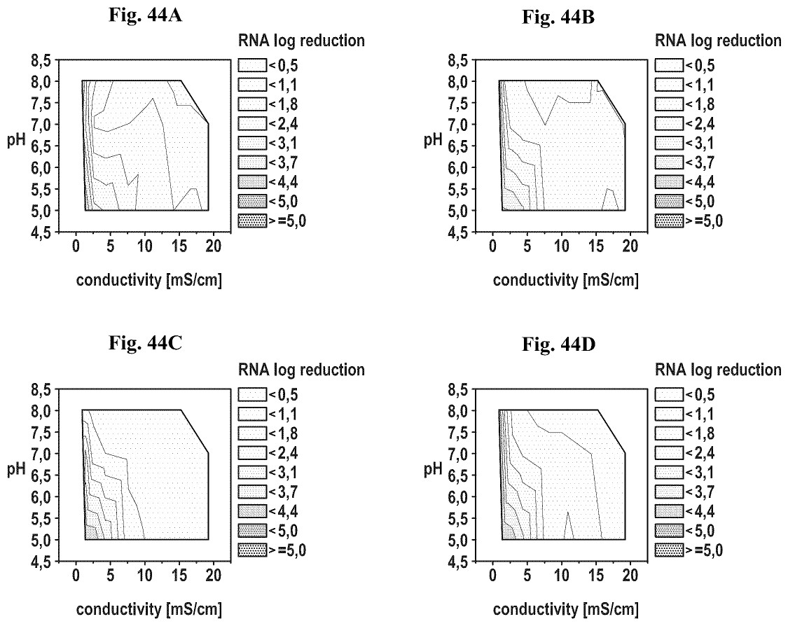

For Example 11 the investigated pH range was pH 5.0-8.0 and the molarity range 25-400 mM Na₂SO₄ (corresponding to a conductivity range of 3-34 mS/cm). For the hydrophilic mabs an improved RVLP reduction was shown compared to the hydrophobic mabs (FIGS. 41A to 42D). For mab2 an RVLP removal of 5 log steps was observed up to a salt molarity of 200 mM Na₂SO₄ (17 mS/cm). To highlight the improved RVLP removal for hydrophilic mabs using an antichaotropic salt, a Tris/Acetate buffer without an antichaotropic salt was investigated additionally (Example 12). For Example 12 the investigated pH range was pH 5.0-8.0 and the molarity range 25-1100 mM Tris/Acetate (corresponding to a conductivity range of 1-19 mS/cm). The contour plots lacking the antichaotropic salt did not show an improved RVLP reduction-neither for the hydrophilic mabs nor for the hydrophobic mabs (FIGS. 43A to 44D). Glossary of used terms:
reference/buffer only: not including any antichaotropic salt but having the same conductivity
load: solution to be loaded independent of composition

LF: load fraction of RC
total load volume: volume required to apply 350 gₚᵣₒₜₑᵢₙ/Lᵣₑₛᵢₙ
total loaded amount: sum of the amount of antibody applied in all applied respective load fractions together
loaded amount: used in calculation of HMW removal with best fit line
HMW value: analytically determined HMW content in a flowthrough fraction
HMW removal value: HMW removal calculated for a single load fraction
HMW removal: calculated HMW removal based on best fit line
pool HMW removal value: calculated HMW removal obtained for a total loaded amount applied in a single fraction
single, pool loaded amount: theoretical amount of antibody loaded in a single fraction of the respective calculated pool HMW removal value
FT: flowthrough fraction
RC: Robocolumn™
RC-run: Robocolumn™ run
CV: column volume

### EXAMPLES

### Part I: Impact of Mab Hydrophobicity on High Molecular Weight (HMW) Impurity Reduction at Constant Conductivity

### Example 1

Robocolumn™ Runs with Loads at pH 8 and 20 mS/Cm

Robotic runs were performed with 4 hydrophilic and 3 hydrophobic mabs at pH 8 and a conductivity of 20 mS/cm. From run to run the buffer condition were varied by adding the following antichaotropic salts: sodium chloride, sodium sulfate, ammonium sulfate, potassium chloride and potassium sulfate. For each run, all 7 mabs were investigated in parallel.

### Buffers

The pH values and conductivities of the respective equilibration buffers are summarized in the following Table X-1.1a. The pH value was each adjusted by adding the respective acid of the buffer (acetic acid). Conductivity was determined after combining all components of the solutions. The buffer 1.5 M Tris/Acetate, pH 8 is the “reference” condition, i.e. not including any antichaotropic salt but having the same conductivity (“buffer only”).

TABLE X-1.1a
Buffer conditions for the RC-runs at pH 8 and 20 mS/cm.

| | | conductivity |
|---|---|---|
| buffer | pH | [mS/cm] |
| 1.5M Tris/Acetate, pH 8 (reference) | 8.09 | 18.71 |
| 70 mM Tris/Acetate, 125 mM Na₂SO₄, pH 8 | 8.03 | 20.70 |
| 70 mM Tris/Acetate, 200 mM NaCl, pH 8 | 7.97 | 21.80 |
| 70 mM Tris/Acetate, 100 mM (NH₄)₂SO₄, pH 8 | 7.90 | 20.20 |
| 70 mM Tris/Acetate, 150 mM KCl, pH 8 | 7.90 | 20.70 |
| 70 mM Tris/Acetate, 100 mM K₂SO₄, pH 8 | 7.98 | 21.50 |

### Concentrating of Starting Material and Buffer Exchange of Antibody Solution

The antibody solutions (mabs) were adjusted to pH and conductivities comparable to the respective buffer. To achieve this the respective affinity column elution pools were buffer exchanged to the respective buffer (e.g. to 70 mM Tris/Acetate, 125 mM Na₂SO₄, pH 8; see Table X-1.1a) and concentrated by using Amicon Ultra Centrifugal Filters. After centrifugation, the mabs were diluted to a concentration of approximately 20 g/L and pH and conductivity were determined. The solutions to be loaded (“loads”) were filtered through a 0.2 μm sterile filter and protein concentration was determined. This procedure was performed for all Robocolumn™ runs (RC-runs).

### Loads

The pH values, conductivities and antibody concentrations of the loads are summarized in the following Table X-1.1b.

TABLE X-1.1b
Load pH, conductivity and concentration for the RC-runs at pH 8 and
20 mS/cm. Ranges are based on the loads comprising the different
antibodies.

| | | conductivity | concentration |
|---|---|---|---|
| | pH range | range of loads | range of loads |
| load in | of loads | [mS/cm] | [g/L] |
| 1.5M Tris/Acetate, pH 8 | 8.03-8.12 | 17.35-17.58 | 19.98-25.17 |
| (reference) | | | |
| 70 mM Tris/Acetate, | 8.04-8.07 | 19.24-19.60 | 18.15-23.69 |
| 125 mM Na₂SO₄, pH 8 | | | |
| 70 mM Tris/Acetate, | 7.91-8.00 | 20.1-20.60 | 19.63-22.94 |
| 200 mM NaCl, pH 8 | | | |
| 70 mM Tris/Acetate, | 7.86-7.89 | 19.38-19.81 | 14.69-20.76 |
| 100 mM (NH₄)₂SO₄, pH 8 | | | |
| 70 mM Tris/Acetate, | 7.89-7.93 | 19.22-19.59 | 18.20-25.02 |
| 150 mM KCl, pH 8 | | | |
| 70 mM Tris/Acetate, | 7.98-7.99 | 19.85-20.20 | 14.93-22.50 |
| 100 mM K₂SO₄, pH 8 | | | |

For all experiments of Example 1, the pH range of the loads was within a range of pH 8.0±0.2. The conductivities of the loads varied ±2.0 mS/cm from the conductivity of the equilibration buffer. The protein concentration of the loads was between 14.5-25.5 g/L.

In the following a description of an exemplary RC-run is provided. All RC-runs were performed alike, except that the buffer for preparing the loads was different (see Tables X-1.1a and b above). For example, for loads using the buffer (70 mM Tris/Acetate, 125 mM Na₂SO₄, pH 8) the pH ranged from pH 8.04-8.07, which means that the load for the 7 different mabs in the equilibration buffer was in this pH range after buffer exchange and concentrating.

The following Table X-1.2 shows the properties of the individual loads for the RC-runs for the different antibodies in 70 mM Tris/Acetate, 125 mM Na₂SO₄, pH 8, flowthrough-mode:

TABLE X-1.2
Example 1 - loads in 70 mM Tris/Acetate, 125 mM Na₂SO₄, pH 8.

| | | | protein |
|---|---|---|---|
| | | conductivity | concentration |
| mab | pH | [mS/cm] | [mg/mL] |
| mab1 | 8.07 | 19.38 | 19.86 |
| mab2 | 8.06 | 19.25 | 22.89 |
| mab4 | 8.04 | 19.25 | 20.77 |
| mab5 | 8.05 | 19.34 | 23.69 |
| mab6 | 8.05 | 19.46 | 19.96 |
| mab7 | 8.05 | 19.24 | 19.15 |
| mab8 | 8.04 | 19.60 | 18.15 |

These loads were individually applied to different Capto adhere ImpRes RCs.

### Chromatography

RC-runs were performed on a Tecan Freedom Evo 150. The RCs were first equilibrated (pH and conductivity adjustment) with 10 CV of buffer_{without} antibody (e.g. 70 mM Tris/Acetate, 125 mM Na₂SO₄, pH 8). Thereafter each RC was loaded stepwise in 200 μL load fractions (LFs) up to 350 gₚᵣₒₜₑᵢₙ/Lᵣₑₛᵢₙ and the flowthrough was collected in 200 μL flowthrough fractions (FT fractions). After the 350 gₚᵣₒₜₑᵢₙ/Lᵣₑₛᵢₙ had been applied, 8 column volumes (CV) of buffer without antibody were applied to the columns to wash residual unbound material from the column before regeneration. The wash following the load was not collected.

The flow rate for all RC-runs was 18 CV/hr which corresponds to a residence time of 3.3 min. The wash was followed by regeneration and storage of the RCs.

TABLE X-1.3
Example 1 - exemplary chromatography steps.

| step | buffer | CV |
|---|---|---|
| equilibration | 70 mM Tris/Acetate, 125 mM Na₂SO₄, pH 8 | 10 |
| load | see Table X-1.2 | 14-20 |
| wash | 70 mM Tris/Acetate, 125 mM Na₂SO₄, pH 8 | 8 |

The following Table X-1.4 summarizes the load concentrations and volumes.

TABLE X-1.4
Example 1-load concentration and
volumes used in RC-runs using the
scheme of Table X-1.3.

| | load | total load |
|---|---|---|
| | concentration | volume |
| mab | [mg/mL] | [μL] |
| mab1 | 19.86 | 3530 |
| mab2 | 22.89 | 3060 |
| mab4 | 20.77 | 3370 |
| mab5 | 23.69 | 2950 |
| mab6 | 19.95 | 3510 |
| mab7 | 19.15 | 3660 |
| mab8 | 18.15 | 3860 |

Pipetting of the load fractions by the Tecan Freedom Evo 150 was executed in a mode that all loads for one joint RC-run were completed at the same time. Therefore the starting point for applying the load varied depending on the antibody concentration in the load. Loads with a higher concentration, e.g. mab5, resulted in lower required total load volumes (and thereby loading steps) and resulted in a later start of the loading steps. For each load step a respective flowthrough fraction (FT) was collected. Thus, for each run a different number of 200 μL fractions were applied, collected and analyzed, respectively (at most 20 fractions for mab8).

The following Table X-1.5 shows the increasing total loaded amount [g/L] after each load step. After at most 20 consecutive load steps a total loaded amount of 350 gₚᵣₒₜₑᵢₙ/Lᵣₑₛᵢₙ had been applied to each RC.

TABLE X-1.5a
Example 1—load step depending total loaded amounts [g/L] for the
runs using the schemes of Tables X-1.3 and X-1.4.

| load step | 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|---|
| mab1 total loaded | 0 | 0 | 13 | 33 | 53 | 72 | 92 | 112 | 132 | 152 | 172 | 192 | 211 | 231 | 251 | 271 | 291 | 311 | 331 | 350 |
| amount [g/L] | | | | | | | | | | | | | | | | | | | | |
| mab2 total loaded | 0 | 0 | 0 | 0 | 7 | 30 | 53 | 76 | 98 | 121 | 144 | 167 | 190 | 213 | 236 | 259 | 282 | 304 | 327 | 350 |
| amount [g/L] | | | | | | | | | | | | | | | | | | | | |
| mab4 total loaded | 0 | 0 | 0 | 18 | 38 | 59 | 80 | 101 | 121 | 142 | 163 | 184 | 205 | 225 | 246 | 267 | 288 | 308 | 329 | 350 |
| amount [g/L] | | | | | | | | | | | | | | | | | | | | |
| mab5 total loaded | 0 | 0 | 0 | 0 | 0 | 18 | 41 | 65 | 89 | 113 | 136 | 160 | 184 | 207 | 231 | 255 | 278 | 302 | 326 | 350 |
| amount [g/L] | | | | | | | | | | | | | | | | | | | | |
| mab6 total loaded | 0 | 0 | 11 | 31 | 51 | 71 | 91 | 111 | 131 | 151 | 171 | 191 | 211 | 230 | 250 | 270 | 290 | 310 | 330 | 350 |
| amount [g/L] | | | | | | | | | | | | | | | | | | | | |
| mab7 total loaded | 0 | 6 | 25 | 44 | 63 | 82 | 101 | 121 | 140 | 159 | 178 | 197 | 216 | 236 | 255 | 274 | 293 | 312 | 331 | 350 |
| amount [g/L] | | | | | | | | | | | | | | | | | | | | |
| mab8 total loaded | 5 | 24 | 42 | 60 | 78 | 96 | 114 | 132 | 151 | 169 | 187 | 205 | 223 | 241 | 260 | 278 | 296 | 314 | 332 | 350 |
| amount [g/L] | | | | | | | | | | | | | | | | | | | | |

### Analytics

TABLE X-1.5b
SE-HPLC analytics of selected FT fractions.

| load step | 1 | 2 | 3 | 4 | 5 | 6 | 7 | 8 | 9 | 10 | 11 | 12 | 13 | 14 | 15 | 16 | 17 | 18 | 19 | 20 | Load |
|---|---|---|---|---|---|---|---|---|---|---|---|---|---|---|---|---|---|---|---|---|---|
| mab1 HMW | | | | | 0.30 | | 0.48 | | | 0.85 | | 1.28 | | 1.54 | | 1.68 | | 1.80 | | 1.93 | 2.93 |
| value [%] | | | | | | | | | | | | | | | | | | | | | |
| mab2 HMW | | | | | | | | | | 1.66 | | 3.60 | | 4.77 | | 5.51 | | 6.12 | | 6.54 | 9.97 |
| value [%] | | | | | | | | | | | | | | | | | | | | | |
| mab4 HMW | | | | | | | 0.58 | | | 0.84 | | 1.04 | | 1.17 | | 1.27 | | 1.44 | | 1.54 | 2.52 |
| value [%] | | | | | | | | | | | | | | | | | | | | | |
| mab5 HMW | | | | | | | | | | 0.74 | | 0.95 | | 0.99 | | 1.04 | | 1.08 | | 1.13 | 1.62 |
| value [%] | | | | | | | | | | | | | | | | | | | | | |
| mab6 HMW | | | | | | | 8.19 | | | 12.63 | | 13.22 | | 13.57 | | 13.71 | | 14.03 | | 14.27 | 16.75 |
| value [%] | | | | | | | | | | | | | | | | | | | | | |
| mab7 HMW | | | | | | | 4.56 | | | 5.55 | | 6.07 | | 6.45 | | 6.89 | | 6.89 | | 7.13 | 9.62 |
| value [%] | | | | | | | | | | | | | | | | | | | | | |
| mab8 HMW | | | | | 3.56 | | 5.02 | | | 5.69 | | 6.2 | | 6.38 | | 6.49 | | 6.77 | | 6.89 | 8.95 |
| value [%] | | | | | | | | | | | | | | | | | | | | | |

TABLE X-1.5c
HMW removal values.

| load step | 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|---|
| mab1 HMW removal | | | | | 89.76 | | 83.62 | | | 70.99 | | 56.31 | | 47.44 | | 42.66 | | 38.57 | | 34.13 |
| value [%] | | | | | | | | | | | | | | | | | | | | |
| mab2 HMW removal | | | | | | | | | | 83.35 | | 63.89 | | 52.16 | | 44.73 | | 38.62 | | 34.40 |
| value [%] | | | | | | | | | | | | | | | | | | | | |
| mab4 HMW removal | | | | | | | 76.98 | | | 66.67 | | 58.73 | | 53.57 | | 49.60 | | 42.86 | | 38.89 |
| value [%] | | | | | | | | | | | | | | | | | | | | |
| mab5 HMW removal | | | | | | | | | | 54.32 | | 41.36 | | 38.89 | | 35.80 | | 33.33 | | 30.25 |
| value [%] | | | | | | | | | | | | | | | | | | | | |
| mab6 HMW removal | | | | | | | 51.10 | | | 24.60 | | 21.07 | | 18.99 | | 18.15 | | 16.24 | | 14.81 |
| value [%] | | | | | | | | | | | | | | | | | | | | |
| mab7 HMW removal | | | | | | | 52.60 | | | 42.31 | | 36.90 | | 32.95 | | 28.38 | | 28.38 | | 25.88 |
| value [%] | | | | | | | | | | | | | | | | | | | | |
| mab8 HMW removal | | | | | 60.22 | | 43.91 | | | 36.42 | | 30.73 | | 28.72 | | 27.49 | | 24.36 | | 23.02 |
| value [%] | | | | | | | | | | | | | | | | | | | | |

The HMW removal value was calculated with the following formula 1:

$$HMW removal value in \% = 100 - {HMW}_{fraction} / {HMW}_{load} \times 100 \% \qquad ( 1 )$$

$${HMW}_{fraction} = HMW value [ \% ] determined in the flowthrough ( FT ) fraction ,$$

$${HMW}_{load} = HMW value [ \% ] determined in the respective load [ \% ] .$$

Example for calculation of the HMW removal value for mab2 in load step 10:
In load step 10 the determined HMW value of the respective FT fraction was 1.66%. The HMW value of the load was 9.97%. Applying this to formula 1

$$HMW removal value = 100 - 1.66 / 9.97 \times 100 \% = 83.35 \%$$

resulted in a HMW removal value of 83.35% for loaded fraction 10 of mab2.

### Analysis

Either the HMW values or mainpeak values or the HMW removal values were plotted against the total loaded amount of antibody.

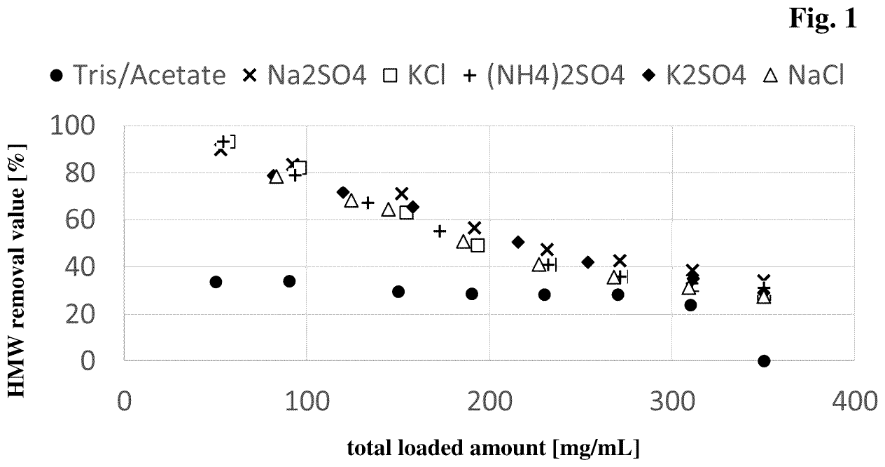

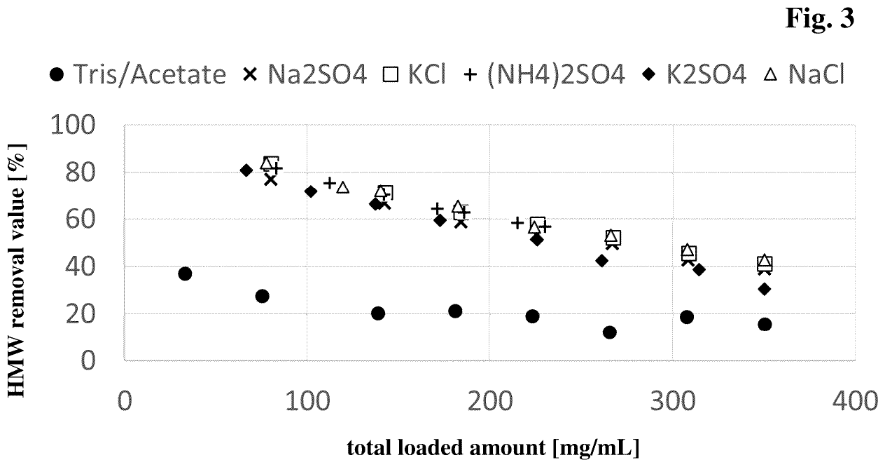

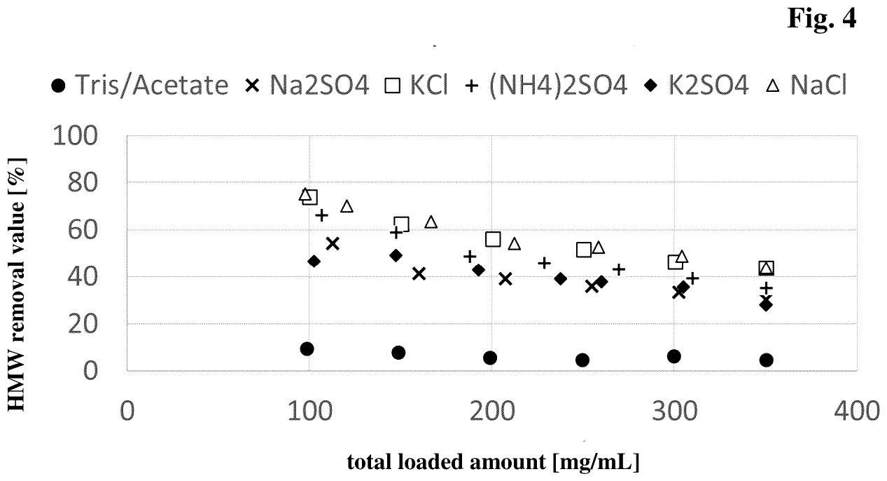

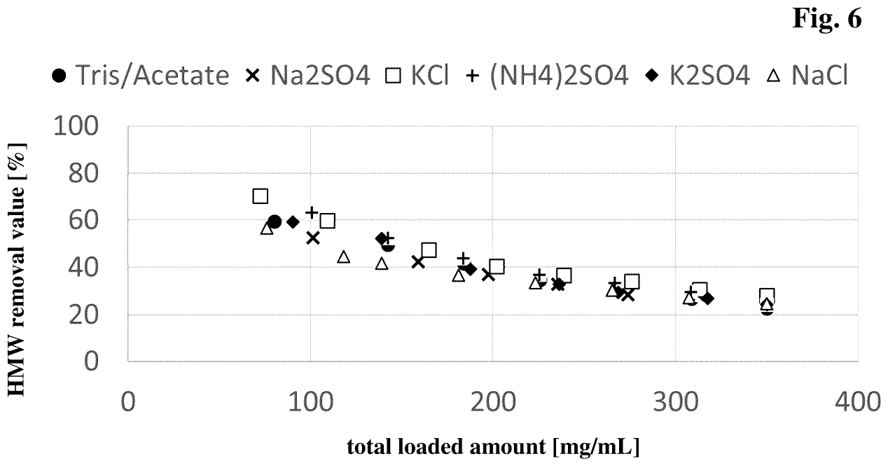

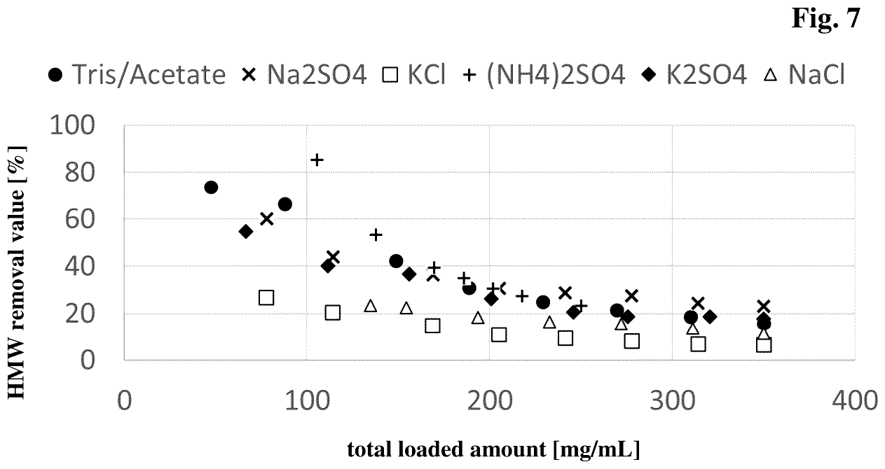

FIGS. 1-7 illustrate the HMW removal values determined for FT fractions of the investigated mabs with the different antichaotropic salts at pH 8.0 and a conductivity of 20 mS/cm. The x-axis corresponds to the total loaded amount, the y-axis corresponds to the HMW removal value as determined for the respective FT fraction.

The HMV removal values in Table X-1.5c show the actual HMW removal that was obtained in the respective load step, i.e. for the corresponding load fraction. However, a pool HMW removal value more closely reflects the actual large-scale process. This pool HMW removal value is the HMW removal obtained for a total loaded amount when applied in a single fraction. Pool HMW removal values were calculated for single, pool load amounts of 150 g/L, 250 g/L 350 g/L, 450 g/L and 550 g/L based on a logarithmic best fit line of the data in Table X-1.5c.

This procedure is explained exemplarily for mab2 below;

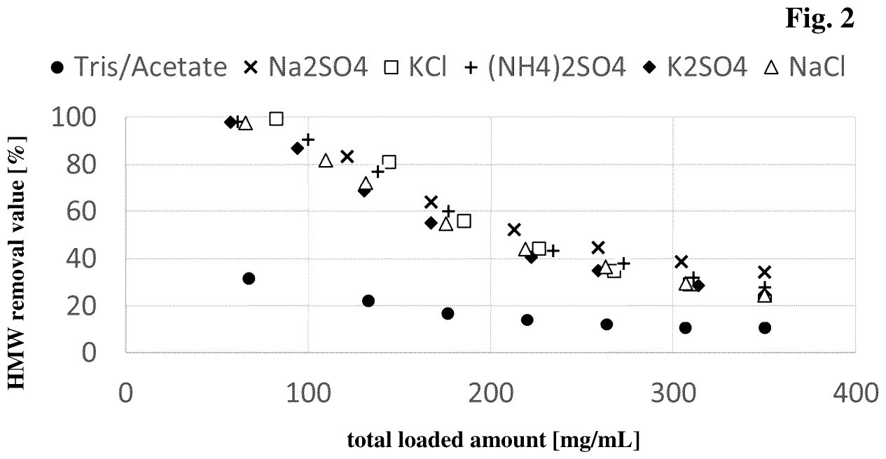

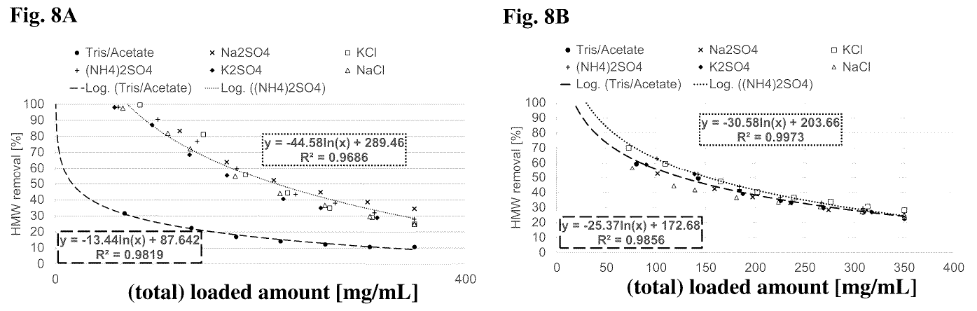

FIG. 2 shows the HMW removal values of each FT fraction for mab2. The results obtained for the different buffers of Table X-1.1 are displayed in this graph. To calculate pool HMW removal values for single, pool loaded amounts of mab2, first, logarithmic best fit lines for the HMW removal of each buffer were determined. The addition of these best fit trend lines for mab2 is shown in FIG. 8A. Second, with the two best fit trend line equations HMW removals were calculated for loaded amounts for 5 g/L and in 25 g/L increments in the range of 25-550 g/L.

For example, to calculate the pool HMW removal value for a single, pool loaded amount of, e.g., 150 g/L, the average was calculated based on the HMW removals of each calculated HMW removal value ≤150 g/L. For small loaded amounts (5-50 g/L) the HMW removal was set to 100% as the calculation resulted in non-logic HMW removals >100%. Table X-1.6 shows the calculated HMW removals.

TABLE X-1.6
Example 1-calculation of HMW removal
and pool HMW removal values.

| | 70 mM Tris/Acetate, | | 1.5M Tris/Acetate, | |
|---|---|---|---|---|
| | 100 mM (NH₄)₂SO₄, pH 8 | | pH 8 | |
| mab 2 | HMW | pool | HMW | pool |
| loaded | removal | HMW | removal | HMW |
| amount | [%] using | removal | [%] using | removal |
| [g/L] | best fit line | value [%] | best fit line | value [%] |
| 5 | 100 | | 66 | |
| 25 | 100 | | 44 | |
| 50 | 100 | | 35 | |
| 75 | 97 | | 30 | |
| 100 | 84 | | 26 | |
| 125 | 74 | | 23 | |
| 150 | 66 | 89 | 20 | 35 |
| 175 | 59 | | 18 | |
| 200 | 53 | | 16 | |
| 225 | 48 | | 15 | |
| 250 | 43 | 75 | 13 | 28 |
| 275 | 39 | | 12 | |
| 300 | 35 | | 11 | |
| 325 | 32 | | 10 | |
| 350 | 28 | 64 | 9 | 23 |
| 375 | 25 | | 8 | |
| 400 | 22 | | 7 | |
| 425 | 20 | | 6 | |
| 450 | 17 | 55 | 6 | 20 |
| 475 | 15 | | 5 | |
| 500 | 12 | | 4 | |
| 523 | 10 | | 3 | |
| 550 | 8 | 47 | 3 | 17 |

Additionally these calculations were performed for one hydrophobic mab (mab7). FIG. 8B displays the respective best fit lines and equations for mab7.

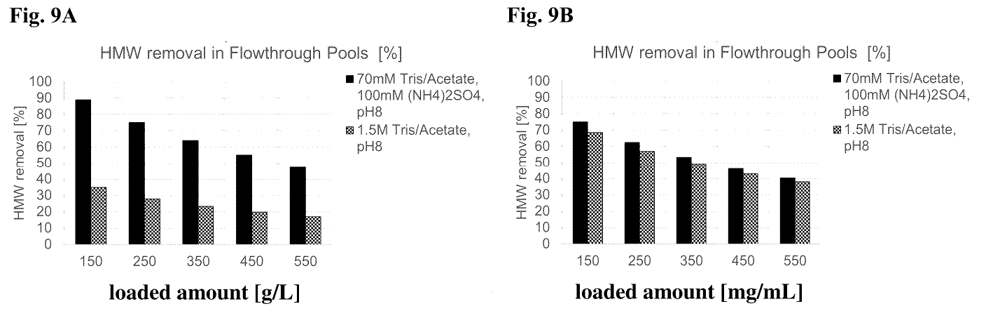

In FIGS. 9A and 9B the HMW removal for single, pool loaded amounts are shown for mab2 (FIG. 9A) and for mab7 (FIG. 9B).

### Summary of Example 1

For hydrophilic mabs (mab1, mab2, mab4, mab5) the HMW value was reduced better when an antichaotropic salt was added to the load compared to a load without an antichaotropic salt at same conductivity. This is illustrated in FIGS. 1 to 4. The conductivity of the loads was comparable (all about 20 mS/cm). The presence of an antichaotropic salt in the load enhanced HMW reduction for hydrophilic mabs while load conductivity was not changed.

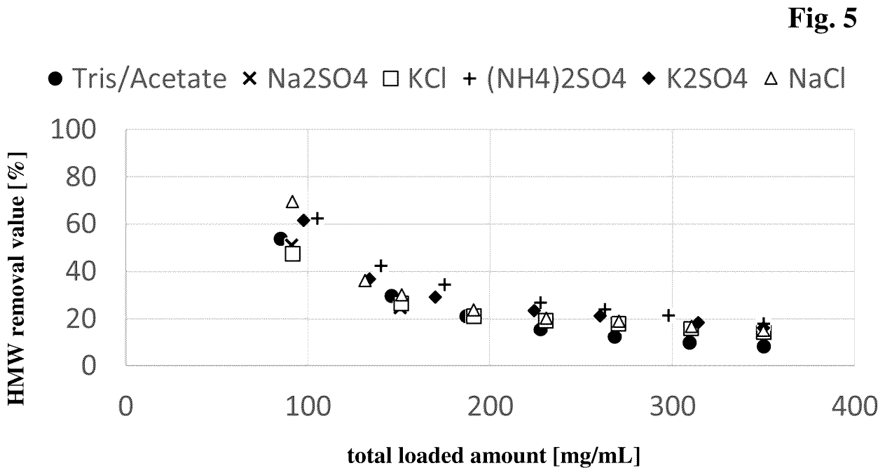

In contrast to that, for the hydrophobic mabs (mab6, mab7, mab8) HMW value reduction was similar for loads containing an antichaotropic salt and for loads without an antichaotropic salt (see FIGS. 5 to 7). For the hydrophobic mabs the addition of an antichaotropic salt showed no advantageous effect with respect to HMW value reduction compared to loads without antichaotropic salt.

To calculate the HMW removal for FT pools, trend lines were introduced. In FIGS. 8A and 8B the HMW removal for the FT pools is shown for mab2 (FIG. 8A) and for mab7 (FIG. 8B). For mab2, for example, it can be seen that HMW reduction at a loaded amount of 150 g/L increased from 35% to 89% when ammonium sulfate was present in the load (see FIG. 9A). For a loaded amount of 550 g/L HMW reduction increased from 17% to 47% in the presence of (NH₄)₂SO₄. In contrast to that and surprisingly, the HMW reduction for mab7 in the presence as well as in the absence of an antichaotropic salt were similar. Thus, for the hydrophobic mab7 HMW reduction was not improved by addition of an antichaotropic salt (see FIG. 9B).

### Example 2

Robocolumn™ Runs with Loads at pH 6 and 20 mS/Cm

RC-runs were performed with 7 mabs at pH 6 and a conductivity of 20 mS/cm using Tris/Citrate buffers in the absence as well as the presence of two antichaotropic salts, i.e. Na₂SO₄ and KCl. The respective references were loads in 1.0 M Tris/Citrate, pH 6 having same conductivity in the absence of any antichaotropic salt.

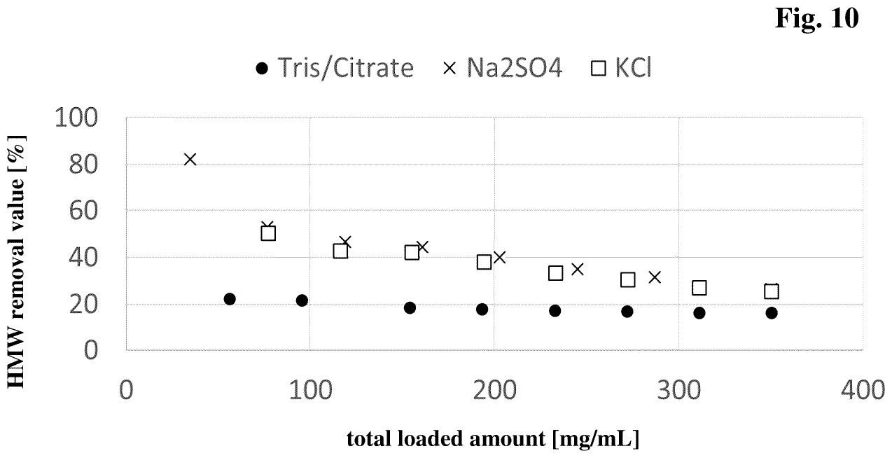

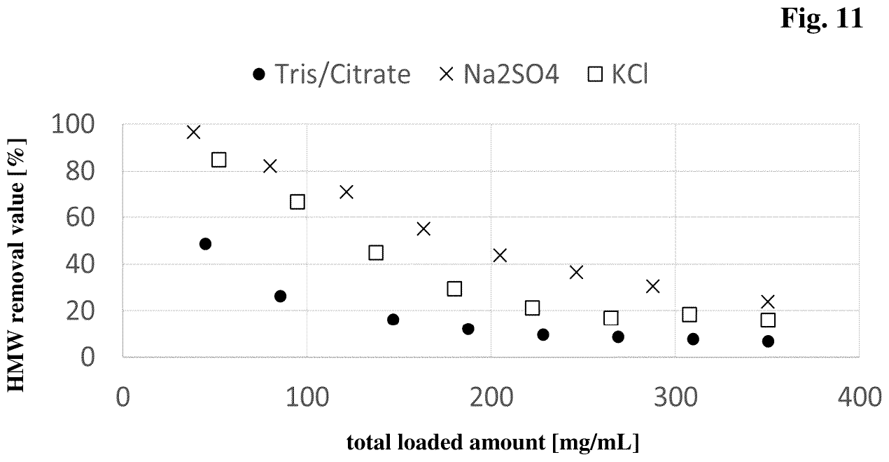

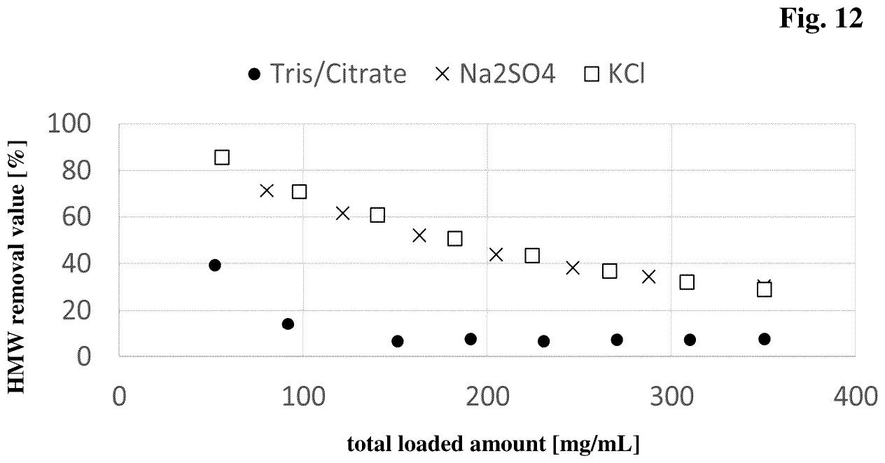

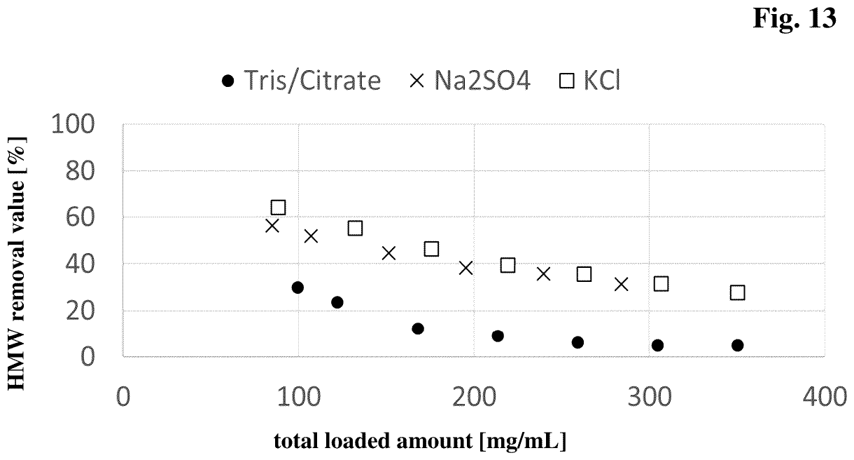

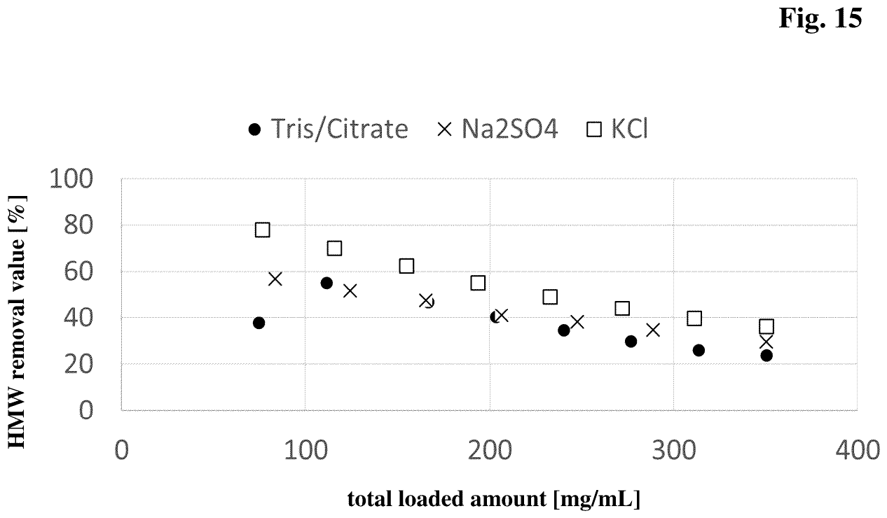

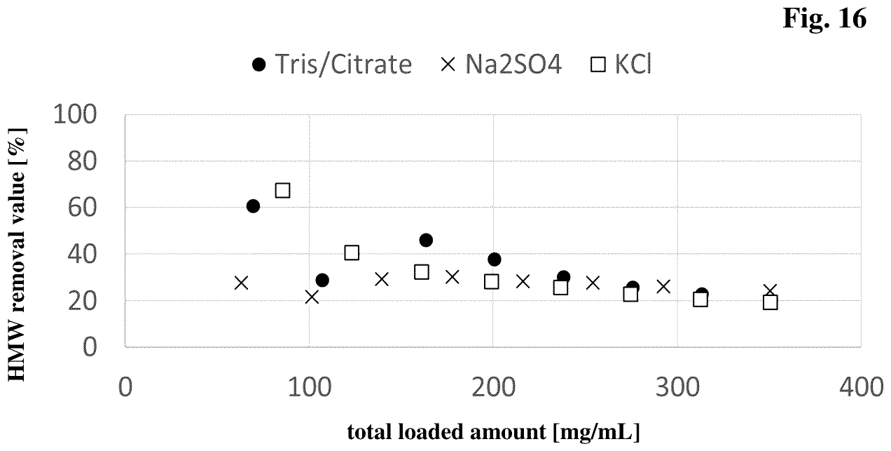

FIGS. 10 to 16 illustrate the HMW removal value of each FT fraction for loads containing Na₂SO₄ and KCl at pH 6.0 and a conductivity of 20 mS/cm. On the x-axis the total loaded amount is displayed.

### Summary of Example 2

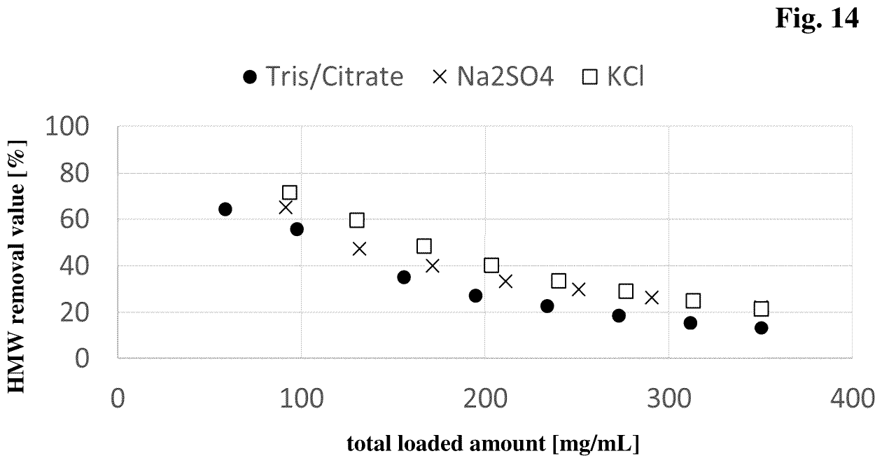

For hydrophilic mabs HMW reduction was significantly improved when an antichaotropic salt was added to the load compared to a load in the same buffer without antichaotropic salt at same conductivity (see FIGS. 10 to 13). In contrast to that and surprisingly, for hydrophobic mabs HMW reduction for loads containing an antichaotropic salt and for loads without an antichaotropic salt was comparable (see FIGS. 14 to 16).

### Example 3

Robocolumn™ Runs with Loads at pH 6 or 8 and 10 mS/Cm

RC-runs were performed with one hydrophilic and one hydrophobic mab in the presence of an antichaotropic salt and in the absence (i.e. without) an antichaotropic salt in the buffer. In both cases, the conductivity of the loads was identical (10 mS/cm). These experiments were performed at pH 6 as well as at pH 8. Up to 350 gₚᵣₒₜₑᵢₙ/Lᵣₑₛᵢₙ were loaded on the RCs. The effect of five antichaotropic salts was analyzed. The runs with a load comprising 400 mM Tris/Acetate, pH 8 and a load comprising 300 mM Tris/Citrate, pH 6 but without antichaotropic salt are the references, respectively.

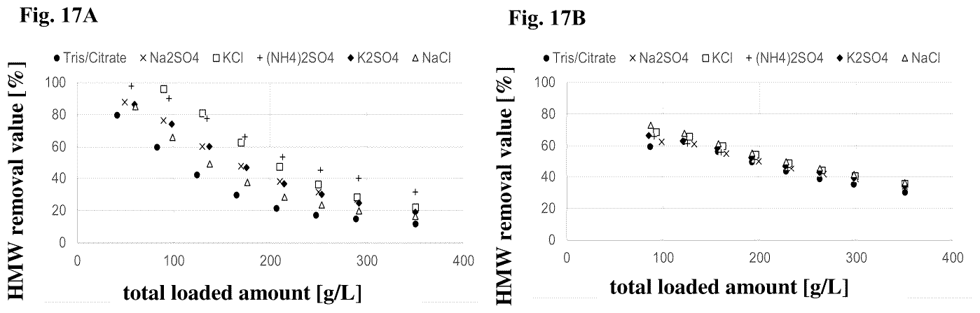

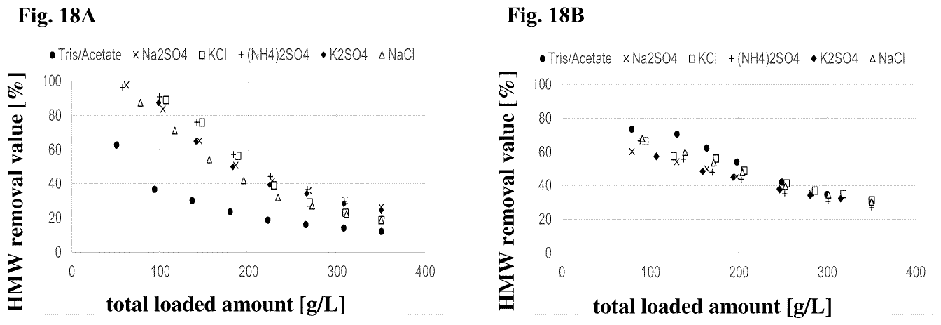

FIGS. 17A and 17B show the HMW removal value of the FT fractions at pH 6 and a conductivity of 10 mS/cm for the hydrophilic mab2 (FIG. 17A), and for the hydrophobic mab7 (FIG. 17B). FIGS. 18A and 18B show the HMW removal value of the FT fractions at pH 8 and a conductivity of 10 mS/cm. FIG. 18A shows the results for the hydrophilic mab2, FIG. 18B shows the results for the hydrophobic mab7.

### Summary of Example 3

FIGS. 17A to 18B show the HMW removal value of each FT fraction in dependence of the total loaded amount for mab2 and mab7 at a conductivity of 10 mS/cm at pH 6 and pH 8, respectively. It can be seen that in the presence of an antichaotropic salt HMW reduction in a load comprising a hydrophilic mab is increased in the FT fractions compared to the reference run without an antichaotropic salt. For the hydrophobic mab, no increased HMW reduction was observed in the presence of an antichaotropic salt.

### Part II: Impact of Antichaotropic Salt Molarity on HMW Reduction

### Example 4

Robocolumn™ Runs with Mab2 at pH 8

RC-runs with mab2 containing loads at pH 8 with antichaotropic salt concentrations in the range of 0-500 mM were performed. The procedure for the RC-runs that was used was already outlined in detail in Example 1. The following salts were used: Na₂SO₄, NaCl, (NH₄)₂SO₄, KCl, K₂SO₄. Flowthrough (FT) fractions were collected and analyzed.

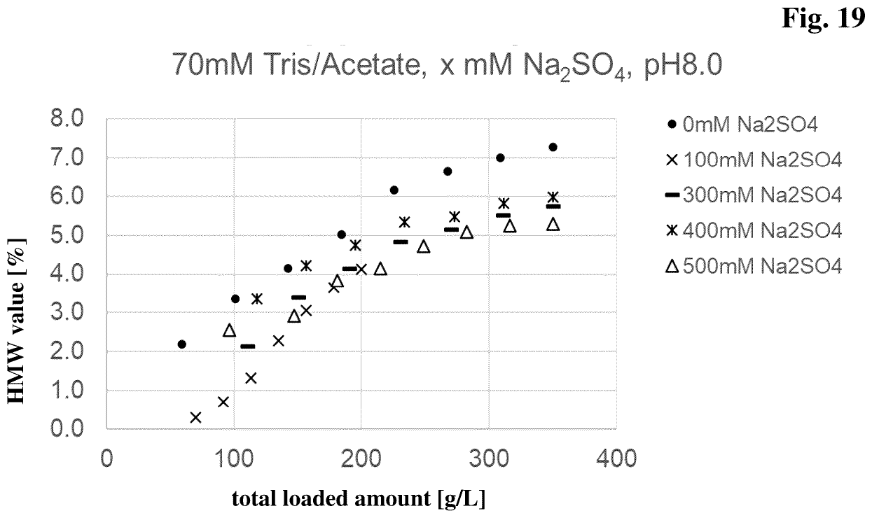

FIGS. 19 to 23 show the HMW values of the FT fractions for mab2 at pH 8 for the different antichaotropic salts.

### Summary of Example 4

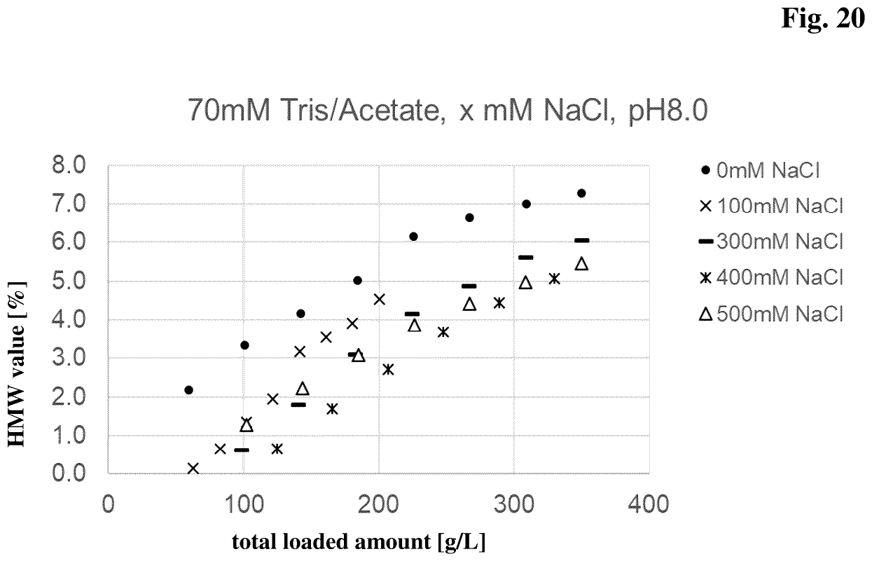

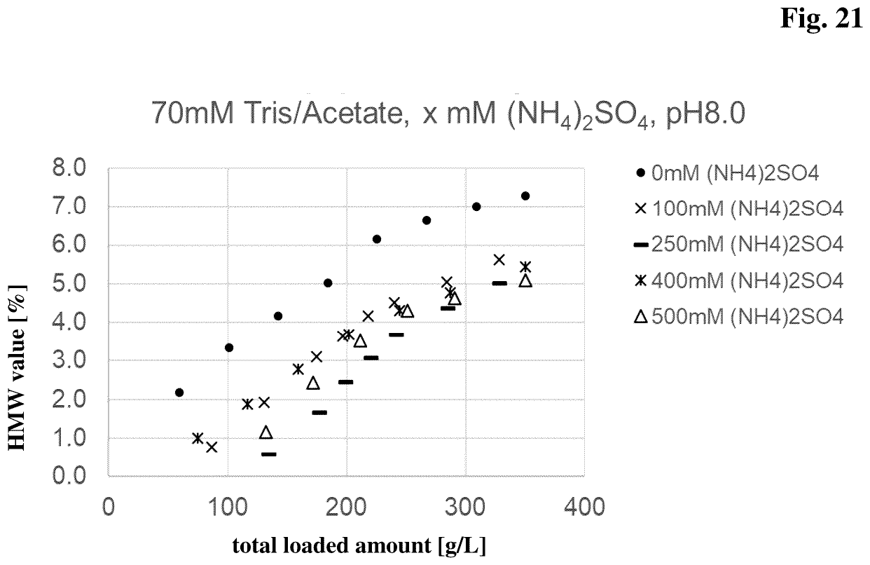

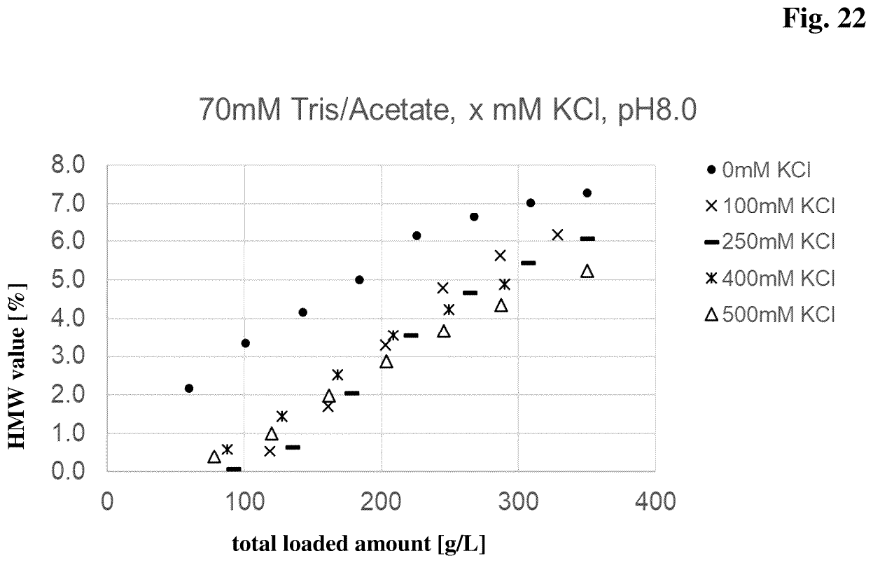

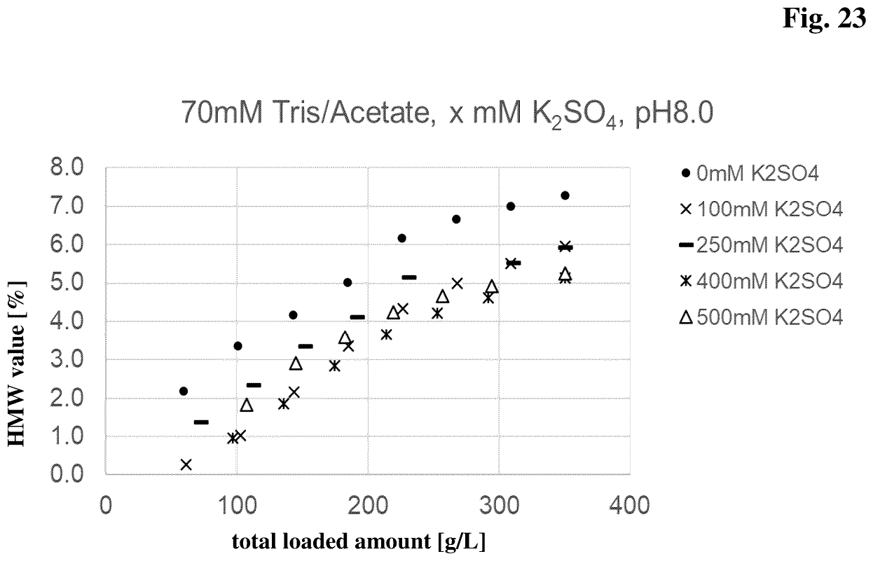

In Example 4 the impact of an increasing antichaotropic salt molarity (and conductivity) was investigated for five antichaotropic salts at pH 8 with hydrophilic mab2. The following antichaotropic salts were used: sodium sulfate (see FIG. 19), sodium chloride (see FIG. 20), ammonium sulfate (see FIG. 21), potassium chloride (see FIG. 22) and potassium sulfate (see FIG. 23). The HMW values [%] of each FT fraction were plotted against the total loaded amount. By adding an antichaotropic salt to the load comprising a hydrophilic mab a reduced HMW value in the FT fractions was achieved. An increased HMW reduction was found for all investigated antichaotropic salts.

The effect of increasing salt molarity was also seen for three mabs (hydrophilic and hydrophobic) and four salts, (NH₄)₂SO₄, KCl, Gua/HCl and Urea, using Kp screens (see Example 5).

### Example 5

### Kp Screens (pH 5.5-8.0)

The methodology of Kp screens is described in detail in this example.

### Preparation of Mab Solutions

The antibody containing solutions were concentrated with Amicon Ultra Centrifugal Filters and buffer exchanged to 10 mM Tris/Acetate, pH 6.5 with Slide-A-Lyzer Dialysis Cassettes. The protein concentrations were in the range of 67 g/L-89 g/L. The total loaded amount for Kp screen was 150 g/L. The loads were 0.2 μm filtered and protein content was determined (OD 280-320).

### Preparation of the Filter Plate

A 50% slurry of the Capto™ adhere ImpRes resin in water was produced in a tube using a centrifuge for rapid settlement. Then the resin was transferred to a shaker placed on the Hamilton Microlab STARlet roboter. Per well of the filter plate 50 μL resin Capto™ adhere ImpRes were added.

### Preparation of Buffers

For the preparation of buffer plate and load plate the following materials were used:
high salt buffers;
low salt buffers;
10 mM Tris/Acetate, pH 6.5 (0.6 mS/cm);
protein stock solution in 10 mM Tris/Acetate, pH 6.5 in an appropriate concentration;
strip buffer.

For Kp screens a buffer plate and a load plate were produced by the robotic system (Tecan Freedom EVO 200) using high salt buffers as well as low salt buffers. The high and low salt stock solutions were prepared by weighing in Tris and the required amount of salt. Then the pH was adjusted with acetic acid.

Table X-2.1 and X-2.2 summarize the low and high salt buffers used to prepare the equilibration and load plates.

TABLE X-2.1
Example 5-low salt buffers for Kp Screen

| | | conductivity |
|---|---|---|
| low salt buffers | pH | [mS/cm] |
| 70 mM Tris/Acetate, pH 5.5 | 5.50 | 3.7 |
| 70 mM Tris/Acetate, pH 7.0 | 7.07 | 3.4 |
| 70 mM Tris/Acetate, pH 8.0 | 8.08 | 2.0 |

TABLE X-2.2
Example 5-high salt buffers for Kp Screen

| | | conductivity |
|---|---|---|
| high salt buffers | pH | [mS/cm] |
| 70 mM Tris/Acetate, | 5.56 | 137.7 |
| 1M (NH₄)₂SO₄, pH 5.5 | | |
| 70 mM Tris/Acetate, | 7.06 | 136.8 |
| 1M (NH₄)₂SO₄, pH 7.0 | | |
| 70 mM Tris/Acetate, | 8.00 | 135.3 |
| 1M (NH₄)₂SO₄, pH 8.0 | | |
| 70 mM Tris/Acetate, | 5.42 | 111.4 |
| 1M KCl, pH 5.5 | | |
| 70 mM Tris/Acetate, | 6.96 | 111.7 |
| 1M KCl, pH 7.0 | | |
| 70 mM Tris/Acetate, | 7.94 | 111.2 |
| 1M KCl, pH 8.0 | | |
| 70 mM Tris/Acetate, | 5.49 | 3.6 |
| 1M Urea, pH 5.5 | | |
| 70 mM Tris/Acetate, | 6.93 | 3.5 |
| 1M Urea, pH 7.0 | | |
| 70 mM Tris/Acetate, | 7.99 | 2.3 |
| 1M Urea, pH 8.0 | | |
| 70 mM Tris/Acetate, | 5.56 | 83.1 |
| 1M Gua/HCl, pH 5.5 | | |
| 70 mM Tris/Acetate, | 6.87 | 83.2 |
| 1M Gua/HCl, pH 7.0 | | |
| 70 mM Tris/Acetate, | 7.89 | 82.9 |
| 1M Gua/HCl, pH 8.0 | | |

The load and equilibration plates were pipetted by the robot as shown in Table X-2.3. The molarities of the four salts were in the range of 10 mM up to ˜800 mM.

TABLE X-2.3
Example 5—plate layout of KpScreen

| | (NH₄)₂SO₄ | | | KCl | | | Urea | | | Gua/HCl | | | salt |
|---|---|---|---|---|---|---|---|---|---|---|---|---|---|
| | pH | pH | pH | pH | pH | pH | pH | pH | pH | pH | pH | pH | molarity |
| | 5.5 | 7.0 | 8.0 | 5.5 | 7.0 | 8.0 | 5.5 | 7.0 | 8.0 | 5.5 | 7.0 | 8.0 | [mM] |
| A | | | | | | | | | | | | | 10 |
| B | | | | | | | | | | | | | 75 |
| C | | | | | | | | | | | | | 150 |
| D | | | | | | | | | | | | | 225 |
| E | | | | | | | | | | | | | 300 |
| F | | | | | | | | | | | | | 450 |
| G | | | | | | | | | | | | | 650 |
| H | | | | | | | | | | | | | ~800 |

The concentrated protein stock solution was pipetted by the robot into the load plate. To neglect a shift in pH and conductivity of each well condition, the protein stock solution was available in 10 mM Tris/Acetate, pH 6.5 and at a high protein concentration to minimize the pipetting volume.

### Execution of Kp Screen

The total loaded amount for the Kp screens was set to 150 g/L and split into two loading steps of each 75 g/L.

The Kp screen method consisted of the following steps:
removal of storage buffer;
equilibration 1+2: transfer of 300 μL equilibration buffer, incubation of 5 min. on Shaker (1100 rpm) and centrifugation to remove the equilibration buffer (2,500 rpm, 600 sec);
loading 1+2: transfer of 300 μL load, incubation of 60 min. on Shaker (1100 rpm) and centrifugation to collect the FT (2,500 rpm, 600 sec) on a FT plate;
strip 1+2: transfer of 300 μL strip buffer, incubation of 5 min. on Shaker (1100 rpm) and centrifugation to remove the strip buffer (2,500 rpm, 600 sec).

SE-HPLC analytics of the FT plate was performed.

The HMW removal was calculated as followed:

$$HMW removal in \% = 100 - {HMW}_{well} / {HMW}_{load} \times 100 \%$$

HMW_{well} is the HMW value [%] measured in a well of the FT plate, HMW_{load} is the HMW level [%] of the protein stock solution.

The protein concentration of the load plate and FT plate were determined using the Infinite M200 plate reader.

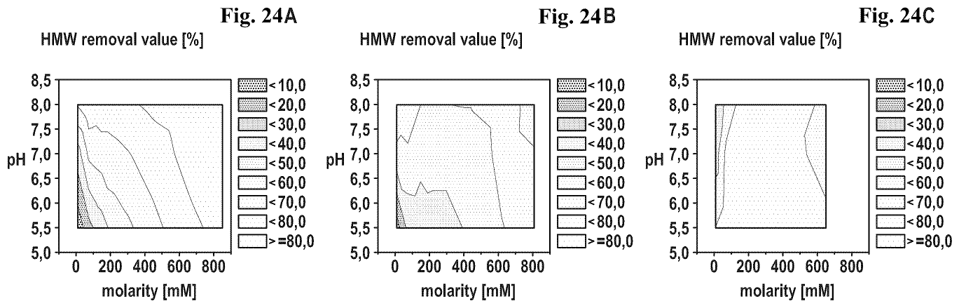

FIGS. 24A to 27C show the HMW removal value [%] of the FT samples for the three mabs and two antichaotropic salts, (NH₄)₂SO₄ and KCl, and two chaotropic salts, Gua/HCl and urea. FIGS. 24A, 25A, 26A and 27A show the HMW removal values for the hydrophilic mab2 and FIGS. 24B, 25B, 26B and 27B show the HMW removal values for the hydrophilic mab4. The HMW removal values for the hydrophobic mab6 are displayed in FIGS. 24C, 25C, 26C and 27C.

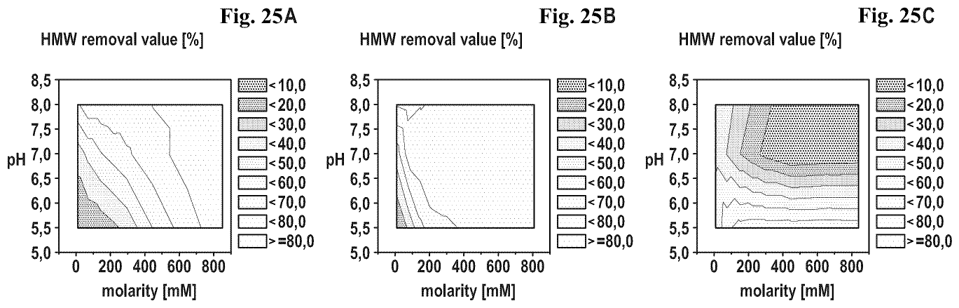

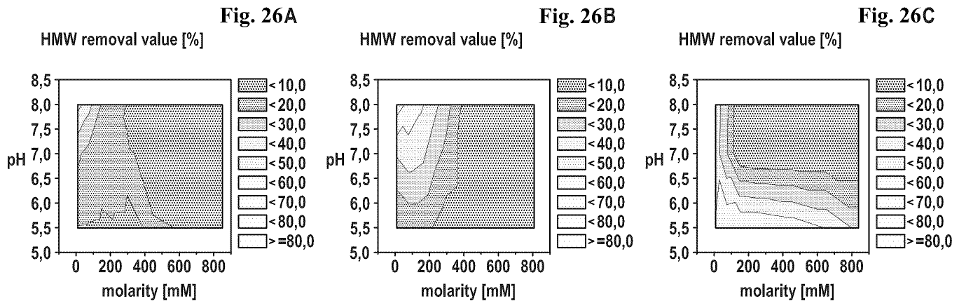

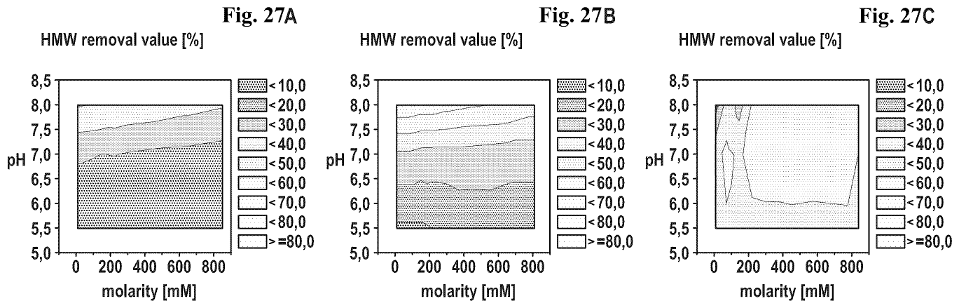

The effect of (NH₄)₂SO₄ on HMW reduction is shown in FIGS. 24A to 24C and the effect of KCl is shown in FIGS. 25A to 25C. The effect of the two chaotropic salts on HMW reduction is shown in FIGS. 26A to 26C (Gua/HCl) and FIGS. 27A to 27C (urea).

### Summary of Example 5

The effect of four salts (two antichaotropic and two chaotropic salts) was shown with two hydrophilic and one hydrophobic mab in the pH range of pH 5.5-8.0 and a salt molarity up to 800 mM. The chaotropic salts (Gua/HCl and urea) were chosen to determine HMW reduction when hydrophobic interactions were weakened.

Depending on the hydrophobicity of the mab, differences in HMW reduction were observed. For the hydrophilic mabs (mab2 and mab4) the addition of an antichaotropic salt (ammonium sulfate, FIGS. 24A to 24C, and KCl, FIGS. 25A to 25C) increased HMW reduction up to 70-80%. For the hydrophobic mab6 HMW reduction in the presence of ammonium sulfate was in the range of 70-80% and nearly unaffected by the ammonium sulfate molarity. With KCl, the HMW reduction for hydrophobic mab6 decreased with increasing KCl molarity. FIGS. 24C and 25C showed that for the hydrophobic mab6 HMW reduction was not improved by increasing molarity of an antichaotropic salt. Gua/HCl (FIGS. 26A to 26C) and urea (FIGS. 27A To 27C) showed that no improvement of HMW removal was observed with increasing salt molarity for both, hydrophilic or hydrophobic mabs.

In summary, HMW reduction was improved for the hydrophilic mabs in the presence of an antichaotropic salt. For the hydrophobic mab, no improved HMW reduction could be seen in the presence of an antichaotropic salt. Furthermore, an improved HMW reduction was not obtained in the presence of chaotropic salts.

### Example 6

Kp Screen with Mab2 (pH 4-9)

For the Kp screen with one hydrophilic mab (mab2) and the two buffer systems i) 25 mM Tris/Acetate comprising (10 to 850) mM Na₂SO₄ and ii) (25 to 975) mM Tris/Acetate the following stock solutions were prepared (see Table X-2.4):

TABLE X-2.4
Example 6-low and high salt buffers for Kp Screen

| | high salt buffers | high salt buffers |
|---|---|---|
| low salt buffers | (Na₂SO₄) | (Tris/Acetate) |
| 25 mM Tris/Acetate, | 25 mM Tris/Acetate, | 1.4M Tris/Acetate, |
| pH 4.0 | 1.4M Na₂SO₄, pH 4.0 | pH 4.0 |
| 25 mM Tris/Acetate, | 25 mM Tris/Acetate, | 1.4M Tris/Acetate, |
| pH 5.0 | 1.4M Na₂SO₄, pH 5.0 | pH 5.0 |
| 25 mM Tris/Acetate, | 25 mM Tris/Acetate, | 1.4M Tris/Acetate, |
| pH 6.0 | 1.4M Na₂SO₄, pH 6.0 | pH 6.0 |
| 25 mM Tris/Acetate, | 25 mM Tris/Acetate, | 1.4M Tris/Acetate, |
| pH 7.0 | 1.4M Na₂SO₄, pH 7.0 | pH 7.0 |
| 25 mM Tris/Acetate, | 25 mM Tris/Acetate, | 1.4M Tris/Acetate, |
| pH 8.0 | 1.4M Na₂SO₄, pH 8.0 | pH 8.0 |
| 25 mM Tris/Acetate, | 25 mM Tris/Acetate, | 1.4M Tris/Acetate, |
| pH 9.0 | 1.4M Na₂SO₄, pH 9.0 | pH 9.0 |

The total loaded amount was 150 g/L and splitted into two loading steps.
Buffer conditions: 2 buffer systems were investigated as shown in Table X-2.5:
25 mM Tris/Acetate+(10-850) mM Na₂SO₄ (pH 4.0-9.0); Na₂SO₄ molarities: 10, 75, 150, 225, 300, 450, 650, 850 mM
25-975 mM Tris/Acetate (pH 4.0-9.0); Tris molarities: 25,100,175, 250, 350, 500, 750, 975 mM

TABLE X-2.5
Example 6—plate layout KpScreen

| | | | | | | | | | | | | | mo- |
|---|---|---|---|---|---|---|---|---|---|---|---|---|---|
| | | | | | | | | | | | | | larity |
| molarity | Na₂SO₄ | | | | | | Tris/Acetate | | | | | | Tris/ |
| Na₂SO₄ | pH | pH | pH | pH | pH | pH | pH | pH | pH | pH | pH | pH | Acetate |
| [mM] | 4.0 | 5.0 | 6.0 | 7.0 | 8.0 | 9.0 | 4.0 | 5.0 | 6.0 | 7.0 | 8.0 | 9.0 | [mM] |
| 10 | | | | | | | | | | | | | 25 |
| 75 | | | | | | | | | | | | | 100 |
| 150 | | | | | | | | | | | | | 175 |
| 225 | | | | | | | | | | | | | 250 |
| 300 | | | | | | | | | | | | | 350 |
| 450 | | | | | | | | | | | | | 500 |
| 650 | | | | | | | | | | | | | 750 |
| 850 | | | | | | | | | | | | | 975 |

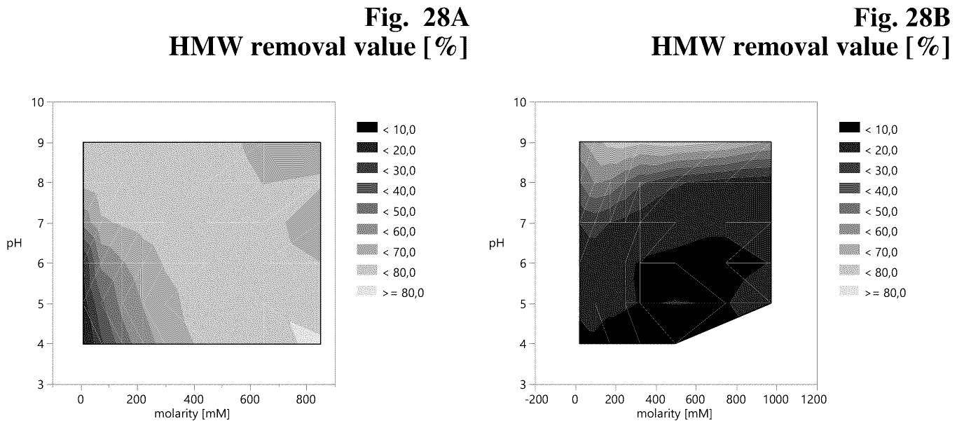

FIGS. 28A to 28B shows the contour plots of the flowthrough for mab2 in the presence of Na₂SO₄ (FIG. 28A) and Tris/Acetate (FIG. 28B).

### Summary of Example 6

Example 6 shows that in the presence of an antichaotropic salt HMW reduction of mab2 containing loads is increased. An increasing Na₂SO₄ molarity (see FIG. 28A) resulted in an improved HMW reduction of up to 80%. The contour plot of mab2 with Na₂SO₄ was similar to that with ammonium sulfate (see FIG. 24A). In contrast to that an increase in Tris/Acetate molarity (see FIG. 28B) had no significant impact on HMW reduction. For Tris/Acetate the HMW reduction was more responsive to changes in pH. Without addition of an antichaotropic salt, no improved HMW reduction was observed with increasing molarity.

Part III: Comparison of HMW Removal with Other Resins/Chromatography Material

### Example 7

Kp Screen with Mixed Mode AEX Resins

The HMW reduction of loads comprising a hydrophilic mab (mab2) and the following mixed mode AEX resins was determined: Capto™ adhere ImpRes, Capto™ adhere and Nuvia aPrime4A. These 3 resins exhibit anionic and hydrophobic moieties.

The Kp screens were executed corresponding Example 5.

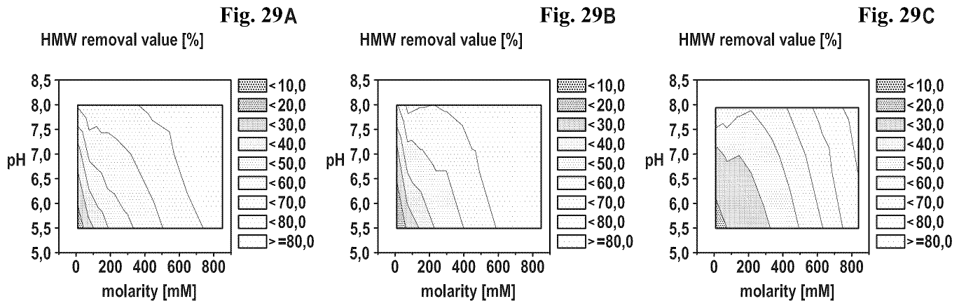

FIGS. 29A to 32C show the contour plots for the three mixed mode resins and four salts (two antichaotropic, two chaotropic).

### Summary of Example 7

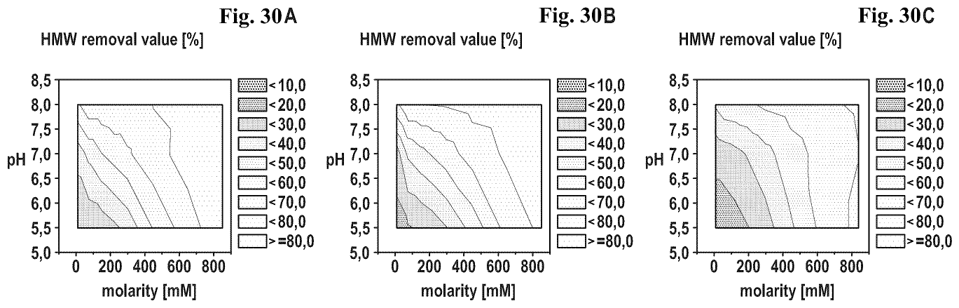

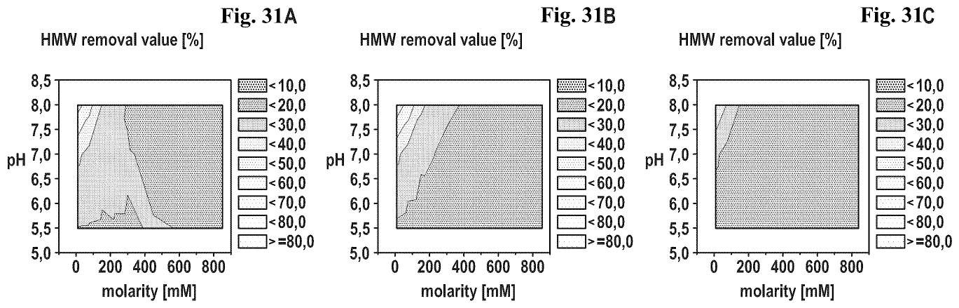

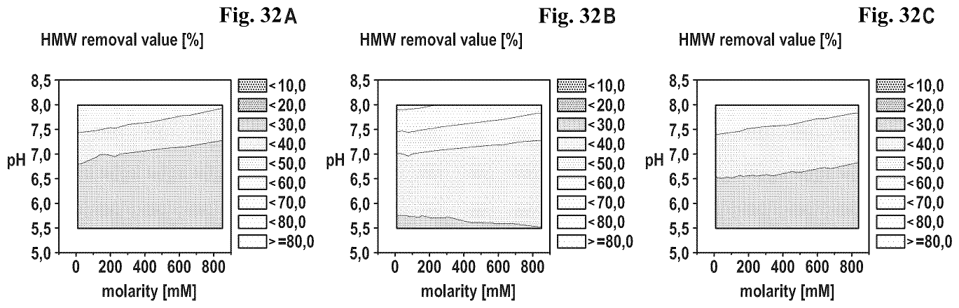

In this Example HMW reduction on three mixed mode resins with anion exchange and hydrophobic interaction were compared. Capto™ adhere ImpRes flowthrough contour plots (FIGS. 29A, 30A, 31A and 32A), Capto™ adhere flowthrough contour plots (FIGS. 29B, 30B, 31B and 32B) and Nuvia aPrime flowthrough contour plots (FIGS. 29C, 30C, 31C and 32C) were generated. Two antichaotropic salts, (NH₄)₂SO₄ (FIGS. 29A to 29C) and KCl (FIGS. 30A to 30C), and two chaotropic salts, Gua/HCl (FIGS. 31A to 31C) and Urea (FIGS. 32A to 32C), were investigated.

In general, for all salts the contour plots of Capto™ adhere, Nuvia aPrime and Capto™ adhere ImpRes showed comparable effects. With increasing (NH₄)₂SO₄ and KCl molarity, all three mixed mode resins showed an improved HMW reduction. The Capto™ adhere, Nuvia aPrime and Capto™ adhere ImpRes contour plots showed good comparability. For the chaotropic salts no improved HMW reduction was observed with the 3 resins.

### Example 8

Kp Screen with MMAEX, AEX, HIC, MMCEX Resins

The contour plots for one hydrophilic mab and one hydrophobic mab were determined with the following resins:
a mixed mode anion exchange resin (Capto™ adhere ImpRes) (MMAEX);
an anion exchange resin (Q Sepharose FF) (AEX);
a hydrophobic resin (Phenyl Sepharose 6 FF (high sub)) (HIC);
a mixed mode cation exchange resin (Capto™ MMC ImpRes) (MMCEX).

The total loaded amount for the resins was 150 g/L except for Capto™ MMC ImpRes with a total loaded amount of 75 g/L.

The Kp screens were executed corresponding Example 5.

For the Kp screen for the Q Sepharose FF, Phenyl Sepharose 6FF (high sub) and Capto™ MMC ImpRes a Na₂SO₄ buffer system was used: 25 mM Tris/Acetate+(5 to 850) mM Na₂SO₄. The following stock solutions were prepared (see X-3.1):

TABLE X-3.1
Example 8-low and high salt buffers

| low salt buffers | high salt buffers |
|---|---|
| 25 mM | 25 mM Tris/Acetate, |
| Tris/Acetate, pH 4.0 | 1.4M Na₂SO₄, pH 4.0 |
| 25 mM | 25 mM Tris/Acetate, |
| Tris/Acetate, pH 5.0 | 1.4M Na₂SO₄, pH 5.0 |
| 25 mM | 25 mM Tris/Acetate, |
| Tris/Acetate, pH 6.0 | 1.4M Na₂SO₄, pH 6.0 |
| 25 mM | 25 mM Tris/Acetate, |
| Tris/Acetate, pH 7.0 | 1.4M Na₂SO₄, pH 7.0 |
| 25 mM | 25 mM Tris/Acetate, |
| Tris/Acetate, pH 8.0 | 1.4M Na₂SO₄, pH 8.0 |
| 25 mM | 25 mM Tris/Acetate, |
| Tris/Acetate, pH 9.0 | 1.4M Na₂SO₄, pH 9.0 |

The following Na₂SO₄ containing buffers were investigated as shown in Table X-3.2:
25 mM Tris/Acetate+ (5-850) mM Na₂SO₄ (pH 4.0-9.0); Na₂SO₄ molarities: 10, 75, 150, 225, 300, 450, 650, 850 mM

TABLE X-3.2
Example 8—plate layout of KpScreen

| molarity | mab2 | | | | | | mab6 | | | | | |
|---|---|---|---|---|---|---|---|---|---|---|---|---|
| Na₂SO₄ | pH | pH | pH | pH | pH | pH | pH | pH | pH | pH | pH | pH |
| [mM] | 4.0 | 5.0 | 6.0 | 7.0 | 8.0 | 9.0 | 4.0 | 5.0 | 6.0 | 7.0 | 8.0 | 9.0 |
| 5 | | | | | | | | | | | | |
| 75 | | | | | | | | | | | | |
| 150 | | | | | | | | | | | | |
| 225 | | | | | | | | | | | | |
| 300 | | | | | | | | | | | | |
| 450 | | | | | | | | | | | | |
| 650 | | | | | | | | | | | | |
| 850 | | | | | | | | | | | | |

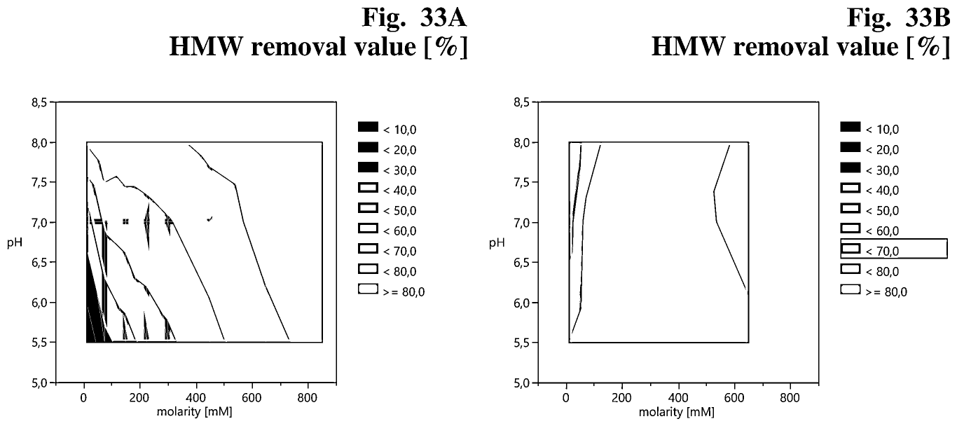

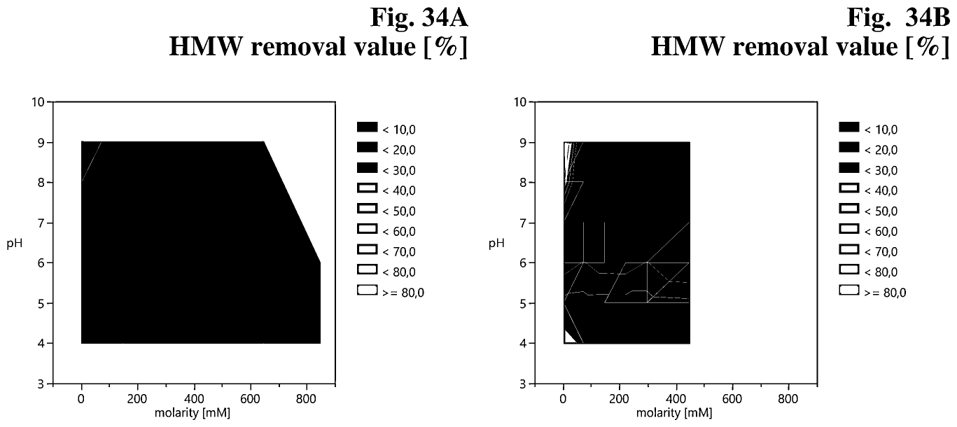

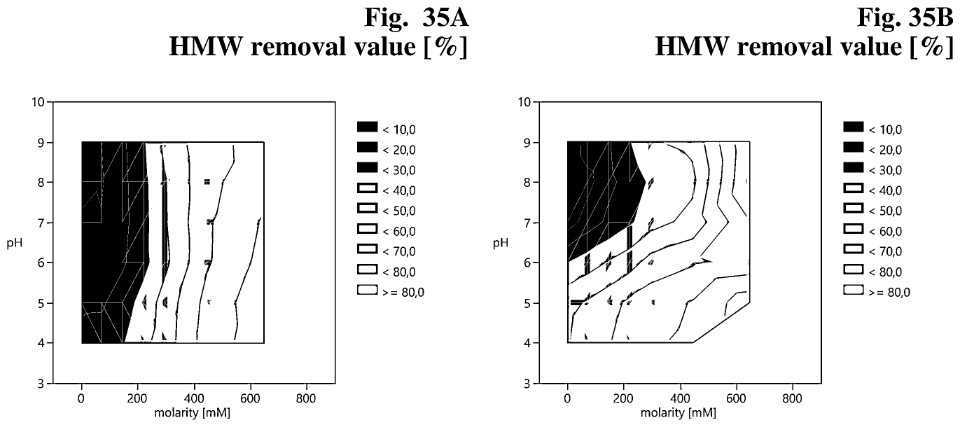

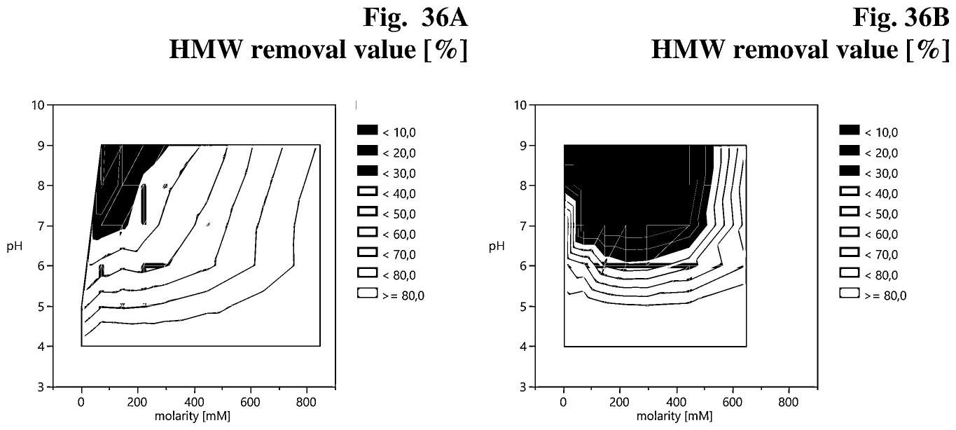

FIGS. 33A to 36B show the contour plots for the hydrophilic mab2 (Figures “A”) and the hydrophobic mab6 (Figures “B”) for the four resins. FIGS. 33A to 33B and 36A to 36B show the HMW reduction for the mixed mode resins Capto™ adhere ImpRes (FIGS. 33A to 33B) and Capto™ MMC ImpRes (FIGS. 36A to 36B); FIGS. 34A to 34B and 35A to 35B show the HMW reduction for the single mode resins Q Sepharose 6FF, an anion exchange resin (FIGS. 34A to 34B) and for Phenyl Sepharose 6 FF (high sub), a hydrophobic resin (FIGS. 35A to 35B).

### Summary of Example 8

With one hydrophilic mab (mab2) and one hydrophobic mab (mab6) the HMW reduction in the flowthrough of the following resins was determined: a mixed mode anion exchange resin (Capto™ adhere ImpRes), an anion exchange resin (Q Sepharose FF), a hydrophobic resin (Phenyl Sepharose 6 FF) and a mixed mode cation exchange resin (Capto™ MMC ImpRes).

For the AEX resin Q Sepharose FF no effect of an antichaotropic salt regarding HMW reduction was observed for both mabs (FIGS. 34A to 34B). In FIGS. 35A to 35B contour plots for the resin Phenyl Sepharose 6FF (high sub) are shown. Both the hydrophilic and hydrophobic mab showed an improved HMW reduction with increasing Na₂SO₄ molarity. Regarding the single mode resins Q Sepharose FF and Phenyl Sepharose 6FF (high sub) the HMW reduction for the hydrophilic and hydrophobic mabs were comparable.

In contrast thereto, for the mixed mode resins HMW reduction for the hydrophilic and hydrophobic mab were different. The MMAEX resin showed an improved HMW reduction for mab2 (FIG. 33A) in the presence of an antichaotropic salt. For mab6 the HMW reduction was quite constant over the investigated pH and molarity range (FIG. 33B). Only for the hydrophilic mab HMW reduction was increased depending on salt molarity on a mixed mode anion exchange resin. FIGS. 36A to 36B show the HMW reduction obtained on the Capto™ MMC ImpRes resin. For the hydrophilic mab HMW reduction was increased with increasing Na₂SO₄ molarity in the range of 0-800 mM from 20% up to 80%. In contrast to that, HMW reduction for the hydrophobic mab was almost unaffected by increasing salt molarity up to 500 mM. For mab6 an improved HMW reduction in the FT samples was observed, but only for molarities of 500 mM or more. Below 500 mM HMW reduction was poor (<10%) and almost independent of salt molarity.

It has been shown that an increased HMW reduction for a hydrophilic mab by addition of an antichaotropic salt could be attributed to the combination of ionic and hydrophobic interaction. For both ionic mixed mode resins, the Capto™ adhere ImpRes (with anionic and hydrophobic moieties) and the Capto™ MMC ImpRes (with cationic and hydrophobic moieties), an increased HMW reduction was achieved with increasing salt molarity, but only for the hydrophilic mab.

### Part IV: ÄKTA Column Runs

### Example 9

Runs at Same Conductivity and Different Na₂SO₄ Molarities

Two loads were prepared with the same conductivity, but different molarities of Na₂SO₄.

Load 1 and load 2 were prepared using the same affinity chromatography pool (column 1 pool) of mab2.
Load 1: A column 1 pool of mab2 was adjusted to pH 8.0 with 1.5 M Tris-base, depth filtered, then conductivity was adjusted to 9 mS/cm using 1 M Na₂SO₄ solution. Then the load was applied to a Capto™ adhere ImpRes column. The conductivity of load 1 was 9 mS/cm, the Na₂SO₄ molarity was 39 mM.
Load 2: A column 1 pool of mab2 was adjusted to pH 8.0 with 1.5 M Tris-base, depth filtered, then pH was adjusted to pH 5.6 with acetic acid, followed by a readjustment to pH 8.0 with 1.5 M Tris. Thereafter conductivity was adjusted to 9 mS/cm with 1 M Na₂SO₄ solution and the solution applied to the column. The conductivity of load 2 was 9 mS/cm, the Na₂SO₄ molarity was 19 mM.

Table X-4.1 summarizes the load adjustment.

TABLE X-4.1
Example 9-load adjustment

| Load 1 | Load 2 |
|---|---|
| (containing ~40 mM | (containing ~20 mM |
| Na₂SO₄ | Na₂SO₄) |
| 1. pH adjustment to pH 8.0 | 1. pH adjustment to pH 8.0 |
| with 1.5M Tris | with 1.5M Tris |
| 2. depth filtration | 2. depth filtration |
| 3. conductivity adjusted | 3. back titration to pH 5.6 |
| to 9 mS/cm | 4. adjustment again to |
| 4. applied to Capto adhere | pH 8.0 |
| ImpRes column | 5. conductivity adjusted |
| | to 9 mS/cm using 1M |
| | Na₂SO₄ |
| | 6. applied to Capto adhere |
| | ImpRes column |
| Load 1 properties | Load 2 properties |
| molarity Na₂SO₄: 39 mM | molarity Na₂SO₄: 19 mM |
| load concentration: 20.34 g/L | load concentration: 18.73 g/L |
| load pH: 8.08 | load pH: 8.00 |
| load conductivity: 9.02 mS/cm | load conductivity: 9.10 mS/cm |

The buffers used in example 9 are listed in Table X-4.2:

TABLE X-4.2
Example 9-buffers

| | | conductivity |
|---|---|---|
| buffer | pH | [mS/cm] |
| 70 mM Tris/Acetate, | 7.96 | 8.73 |
| 40 mM Na₂SO₄, pH 8 | | |
| 1.5M Tris | 11.02 | 0.196 |
| 1M Na₂SO₄ | 5.88 | 93.8 |

The column volume of the Capto™ adhere ImpRes column was 6.84 mL with a column diameter of 0.66 cm. After equilibration of the column with 70 mM Tris/Acetate, 40 mM Na₂SO₄, pH 8 the load was applied to the column. The load capacity was ˜150 g/l and the flow rate was 150 cm/h. The Capto™ adhere ImpRes FT was fractionated. Protein concentration of the fractions was measured and SE-HPLC was performed.

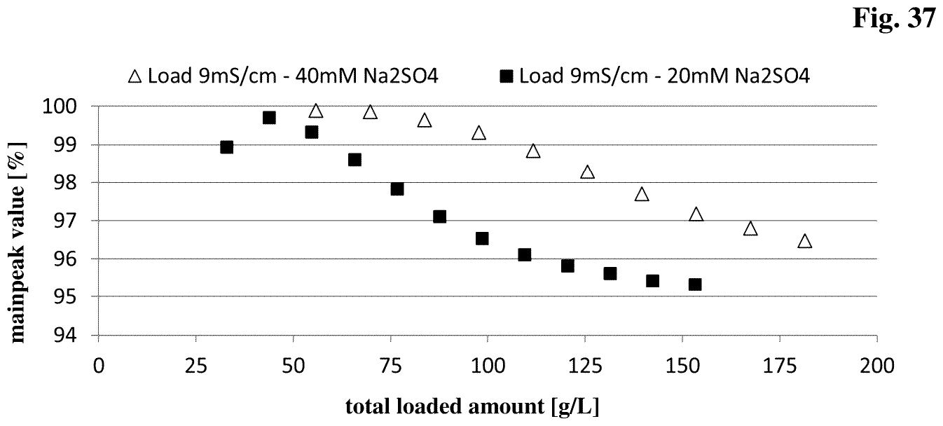

FIG. 37 shows the impact of the Na₂SO₄ molarity on the mainpeak value of the FT fractions for a conductivity of 9 mS/cm.

### Summary of Example 9

Two loads of mab2 were prepared with the same conductivity of 9 mS/cm, but different molarities of Na₂SO₄ (about 40 mM and about 20 mM). The FT fractions of the load with the higher Na₂SO₄ molarity had higher mainpeak values compared to the load with lower Na₂SO₄ molarity (see FIG. 37). This shows that the higher Na₂SO₄ molarity enhanced HMW removal as the load conductivity was equal.

### Example 10

Runs at Different Na₂SO₄ Molarities and Conductivities

The impact of increasing salt molarity (and conductivity) was determined with mab2. Column runs were performed at pH 7 and pH 8 with different Na₂SO₄ molarities and conductivities on a Capto™ adhere ImpRes column (column volume=6.84 mL; d=0.66 cm). The FT was fractionated and the load capacity was ˜150 g/L.

The chromatographic conditions are shown in Table X-4.3:

TABLE X-4.3
Example 10 - chromatography steps

| step | buffer | CV | flow [cm/h] |
|---|---|---|---|
| Equilibration | 70 mM Tris/Acetate, | 7 | 150 |
| | x mM Na₂SO₄, (pH 7/pH 8) | | |
| Load | ~150 g/L | | 150 |

TABLE X-4.4
Example 10-buffers

| | | buffer |
|---|---|---|
| | | conductivity |
| buffer | pH | [mS/cm] |
| 70 mM Tris/Acetate, pH 8.0 | 7.96 | 2.4 |
| 70 mM Tris/Acetate, | 8.04 | 5.7 |
| 20 mM Na₂SO₄, pH 8.0 | | |
| 70 mM Tris/Acetate, | 8.00 | 8.9 |
| 40 mM Na₂SO₄, pH 8.0 | | |
| 70 mM Tris/Acetate, | 8.00 | 11.8 |
| 60 mM Na₂SO₄, pH 8.0 | | |
| 70 mM Tris/Acetate, | 6.92 | 9.8 |
| 40 mM Na₂SO₄, pH 7.0 | | |
| 70 mM Tris/Acetate, | 6.95 | 12.5 |
| 60 mM Na₂SO₄, pH 7.0 | | |
| 1.5M Tris | 11.02 | 0.2 |
| 1M Na₂SO₄ | 5.88 | 93.8 |

The following loads were obtained after adjustment with 1.5 M Tris and 1 M Na₂SO₄ (see Table X-4.5):

TABLE X-4.5
Example 10—load conditions

| | Run 1 | Run 2 | Run 3 | Run 4 | Run 5 | Run 6 |
|---|---|---|---|---|---|---|
| load pH | 8.0 | 8.0 | 8.1 | 8.1 | 7.1 | 7.1 |
| load conductivity | 4.9 | 6.2 | 9.2 | 12.3 | 9.2 | 12.1 |
| [mS/cm] | | | | | | |
| load concentration | 10.7 | 24.5 | 23.3 | 21.8 | 23.6 | 23.5 |
| [mg/mL] | | | | | | |
| load Na₂SO₄ | 0 | 10 | 35 | 60 | 34 | 55 |
| molarity [mM] | | | | | | |

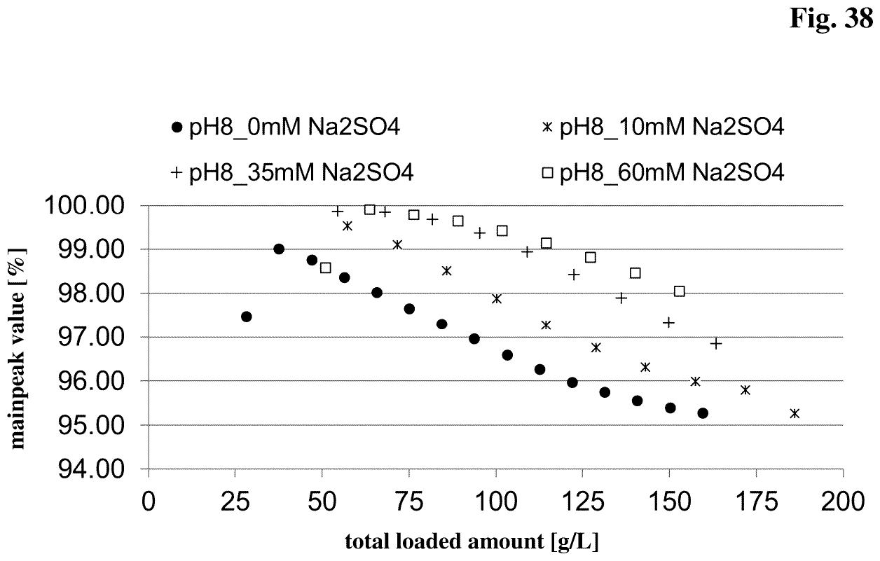

FIG. 38 shows the mainpeak values of each fraction with progressing total loaded amount at pH 8. With increasing Na₂SO₄ molarity from 0 mM to 60 mM the mainpeak value of the FT fractions was improved.

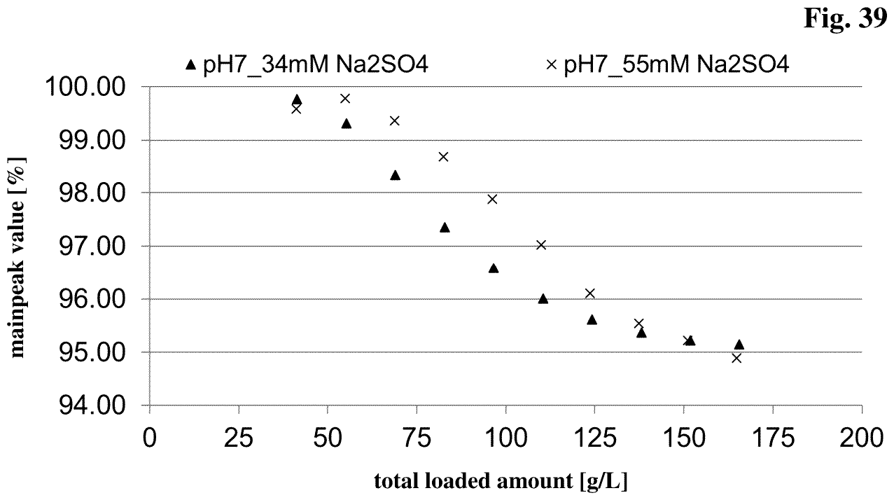

FIG. 39 shows the mainpeak value of each fraction with progressing total loaded amount at pH 7. The curves at pH 7 show that even a small increase of Na₂SO₄ molarity from 34 mM (9 mS/cm) to 55 mM (12 mS/cm) had a positive effect on the mainpeak value of the FT fractions. Corresponding to Kp screen and RC data the mainpeak values in the FT fractions at pH 7 were lower compared to pH 8.

Pools were calculated in the following way:

The mainpeak value of pools was calculated using the average mainpeak value of the fractions. Wash fractions (following the load step) were not included in the FT pools. Table X-4.6 summarizes the run conditions and mainpeak values.

TABLE X-4.6
Example 10-run conditions and
mainpeak values of FT pools

| | conductivity | load molarity | loaded | mainpeak |
|---|---|---|---|---|
| | load | Na₂SO₄ | amount | (FT Pool) |
| pH | [mS/cm] | [mM] | [g/L] | [%] |
| 8.0 | 5 | 0 | 159 | 96.96 |
| 8.0 | 6 | 10 | 157 | 97.67 |
| 8.0 | 9 | 35 | 163 | 98.69 |
| 8.0 | 12 | 60 | 153 | 99.09 |
| 7.0 | 9 | 34 | 166 | 96.87 |
| 7.0 | 12 | 55 | 165 | 97.41 |

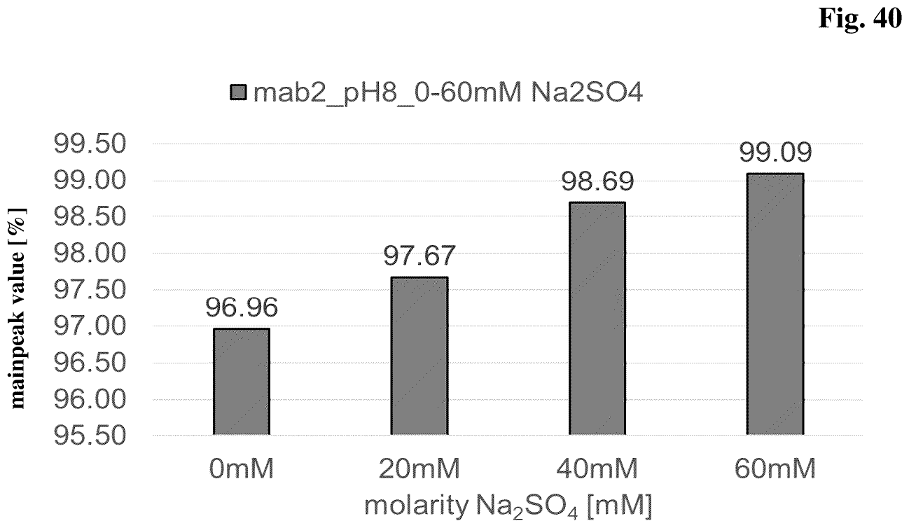

FIG. 40 shows the calculated mainpeak of the FT pools. The mainpeak values were increased from ˜97% (without Na₂SO₄) to ˜99% by adding 60 mM Na₂SO₄ to the load.

### Summary of Example 10

The effect of Na₂SO₄ molarity on HMW removal for pH 7 and pH 8 has been shown. These data support the data obtained with the robotic systems. The mainpeak values of the FT fractions raised with increasing Na₂SO₄ molarity at pH 7 (see FIG. 39) and pH 8 (see FIG. 38). FT pools were calculated using the average mainpeak value of the fractions at pH 8 (see FIG. 40). For pH 8 the mainpeak value was increased from 96.96% (without Na₂SO₄; conductivity of 5 mS/cm) up to 99.09% with a load containing 60 mM Na₂SO₄ (conductivity of 12 mS/cm).

### Examples 11 and 12

### KpScreen: Impact of Antichaotropic Salt and Mab Hydrophobicity on RVLP Removal

### Kp Screens (pH 5.0-8.0)

The methodology of Kp screens in connection with RVLP removal is described in detail in this example.

### Preparation of Mab Solutions

The antibody containing solutions (column 2 pools) were concentrated with Amicon Ultra Centrifugal Filters and buffer exchanged to 10 mM Tris/Acetate, pH 6.5 and concentrated with Amicon Ultra Centrifugal Filters. The protein concentrations were in the range of 61 g/L-72 g/L. The total loaded amount for Kp screen was 150 g/L. The loads were 0.2 μm filtered and protein content was determined (OD 280-320).

### Preparation of the Filter Plate

A 50% slurry of the Capto™ adhere ImpRes resin in water was produced in a tube using a centrifuge for rapid settlement. Then the resin was transferred to a shaker placed on the Hamilton Microlab STARlet roboter. Per well of the filter plate 50 μL resin Capto™ adhere ImpRes were added.

### Preparation of Buffers

For the preparation of buffer plate and load plate the following materials were used:
high salt buffers;
low salt buffers;
10 mM Tris/Acetate, pH 6.5 (0.6 mS/cm);
protein stock solution in 10 mM Tris/Acetate, pH 6.5 in an appropriate concentration;
strip buffer.

For Kp screens a buffer plate and a load plate were produced by the robotic system (Tecan Freedom EVO 200) using high salt buffers as well as low salt buffers. The high and low salt stock solutions were prepared by weighing in Tris and the required amount of salt. Then the pH was adjusted with acetic acid.

Table X-5.1 and X-5.2 summarize the low and high salt buffers used to prepare the equilibration and load plates for Example 11.

TABLE X-5.1
Example 11-low salt buffers for Kp Screen

| | | conductivity |
|---|---|---|
| low salt buffers | pH | [mS/cm] |
| 25 mM Tris/Acetate, pH 5.0 | 5.01 | 1.52 |
| 25 mM Tris/Acetate, pH 7.0 | 7.02 | 1.42 |
| 25 mM Tris/Acetate, pH 8.0 | 8.01 | 0.83 |

TABLE X-5.2
Example 11-high salt buffers for Kp Screen

| | | conductivity |
|---|---|---|
| high salt buffers | pH | [mS/cm] |
| 25 mM Tris/Acetate, | 5.01 | 111.6 |
| 1.4M Na₂SO₄, pH 5.0 | | |
| 25 mM Tris/Acetate, | 6.96 | 116.7 |
| 1.4M Na₂SO₄, pH 7.0 | | |
| 25 mM Tris/Acetate, | 8.00 | 111.1 |
| 1.4M Na₂SO₄, pH 8.0 | | |

Table X-5.3 and X-5.4 summarize the low and high salt buffers used to prepare the equilibration and load plates for Example 12.

TABLE X-5.3
Example 12-low salt buffers for Kp Screen

| | | conductivity |
|---|---|---|
| low salt buffers | PH | [mS/cm] |
| 25 mM Tris/Acetate, pH 5.0 | 5.01 | 1.52 |
| 25 mM Tris/Acetate, pH 7.0 | 7.02 | 1.42 |
| 25 mM Tris/Acetate, pH 8.0 | 8.01 | 0.83 |

TABLE X-5.4
Example 12-high salt buffers for Kp Screen

| | | conductivity |
|---|---|---|
| high salt buffers | pH | [mS/cm] |
| 1.4M Tris/Acetate, pH 5.0 | 5.02 | 111.6 |
| 1.4M Tris/Acetate, pH 7.0 | 7.03 | 116.7 |
| 1.4M Tris/Acetate, pH 8.0 | 8.04 | 111.1 |

Before pipetting the high salt and low salt buffers, 10 μl of an RVLP stock solution were pipetted to each well of the load plate by the robot for Example 11 and 12. The load and equilibration plates were pipetted by the robot as shown in Table X-5.5. For Example 11 the molarities of sodium sulfate were in the range of 25 mM up to 400 mM.

TABLE X-5.5
Example 11—plate layout of KpScreen

| | mab1 | | | mab2 | | | mab9 | | | mab7 | | | Na₂SO₄ |
|---|---|---|---|---|---|---|---|---|---|---|---|---|---|
| | pH | pH | pH | pH | pH | pH | pH | pH | pH | pH | pH | pH | molarity |
| | 5.0 | 7.0 | 8.0 | 5.0 | 7.0 | 8.0 | 5.0 | 7.0 | 8.0 | 5.0 | 7.0 | 8.0 | [mM] |
| A | | | | | | | | | | | | | 25 |
| B | | | | | | | | | | | | | 40 |
| C | | | | | | | | | | | | | 50 |
| D | | | | | | | | | | | | | 75 |
| E | | | | | | | | | | | | | 100 |
| F | | | | | | | | | | | | | 200 |
| G | | | | | | | | | | | | | 300 |
| H | | | | | | | | | | | | | 400 |

For Example 12 the load and equilibration plates were pipetted by the robot as shown in Table X-5.6. For example 12 the molarities of Tris were in the range of 25 mM up to 1100 mM.

TABLE X-5.6
Example 12—plate layout of KpScreen

| | mab1 | | | mab2 | | | mab9 | | | mab7 | | | Tris |
|---|---|---|---|---|---|---|---|---|---|---|---|---|---|
| | pH | pH | pH | pH | pH | pH | pH | pH | pH | pH | pH | pH | molarity |
| | 5.0 | 7.0 | 8.0 | 5.0 | 7.0 | 8.0 | 5.0 | 7.0 | 8.0 | 5.0 | 7.0 | 8.0 | [mM] |
| A | | | | | | | | | | | | | 25 |
| B | | | | | | | | | | | | | 100 |
| C | | | | | | | | | | | | | 200 |
| D | | | | | | | | | | | | | 400 |
| E | | | | | | | | | | | | | 600 |
| F | | | | | | | | | | | | | 800 |
| G | | | | | | | | | | | | | 1000 |
| H | | | | | | | | | | | | | 1100 |

For Example 11 and 12 the concentrated protein stock solution was pipetted by the robot into the load plate. To neglect a shift in pH and conductivity of each well condition, the protein stock solution was available in 10 mM Tris/Acetate, pH 6.5 and at a high protein concentration to minimize the pipetting volume.

### Execution of Kp Screen

The total loaded amount for the Kp screens was set to 150 g/L and split into two loading steps of each 75 g/L.

The Kp screen method consisted of the following steps:
removal of storage buffer;
equilibration 1+2: transfer of 300 μL equilibration buffer, incubation of 5 min. on Shaker (1100 rpm) and centrifugation to remove the equilibration buffer (2,500 rpm, 600 sec);
loading 1+2: transfer of 300 μL load, incubation of 60 min. on Shaker (1100 rpm) and centrifugation to collect the FT (2,500 rpm, 600 sec) on a FT plate;
strip 1+2: transfer of 300 μL strip buffer, incubation of 5 min. on Shaker (1100 rpm) and centrifugation to remove the strip buffer (2,500 rpm, 600 sec).

RNA analytics were performed for the FT plate.

The RVLP removal (RNA log reduction) was calculated with the RNA concentrations as followed:

$$RNA log reduction = Log ( RNA {concentration}_{load} / RNA {concentration}_{well} )$$

RNA concentration_{load} is the average RNA concentration [copies/μL] measured in selected wells of the load plate. For Example 11 the average RNA concentration of the load wells was 182357 [copies/μL]. For Example 12 the average RNA concentration of the load wells was 84250 [copies/μL].

RNA concentration_{well} is the RNA concentration [copies/μL] measured in each well of the FT plate for Example 11 and 12.

FIGS. 41A to 41D show the RNA log reduction of the FT samples for four mabs and the antichaotropic salt Na₂SO₄ with increasing sodium sulfate molarity. FIGS. 41A and 41B show the RNA log reduction for the hydrophilic mab1 (FIG. 41A) and mab2 (FIG. 41B).

FIGS. 41C and 41D show the RNA log reduction for the hydrophobic mab7 (FIG. 41C) and mab9 (FIG. 41D).

FIGS. 42A to 42D show the RNA log reduction of the FT samples for four mabs and the antichaotropic salt Na₂SO₄ with increasing conductivity. FIGS. 42A and 42B show the RNA log reduction for the hydrophilic mab1 (FIG. 42A) and mab2 (FIG. 42B). FIGS. 42C and 42D show the RNA log reduction for the hydrophobic mab7 (FIG. 42C) and mab9 (FIG. 42D).

FIGS. 43A to 43D show the RNA log reduction of the FT samples for four mabs in a Tris/Acetate buffer without an antichaotropic salt with increasing Tris molarity. FIGS. 43A and 43B show the RNA log reduction for the hydrophilic mab1 (FIG. 43A) and mab2 (FIG. 43B). FIGS. 43C and 43D show the RNA log reduction for the hydrophobic mab7 (FIG. 43C) and mab9 (FIG. 43D).

FIGS. 44A to 44D show the RNA log reduction of the FT samples for four mabs in a Tris/Acetate buffer without an antichaotropic salt with increasing conductivity. FIGS. 44A and 44B show the RNA log reduction for the hydrophilic mab1 (FIG. 44A) and mab2 (FIG. 44B). FIGS. 44C and 44D show the RNA log reduction for the hydrophobic mab7 (FIG. 44C) and mab9 (FIG. 44D).

### Summary of Example 11 and 12

The effect of mab hydrophobicity and the presence of an antichaotropic salt was shown with two hydrophilic and two hydrophobic mabs in the pH range of pH 5.0-8.0. In Example 11 (FIGS. 41A to 42D) the antichaotropic salt sodium sulfate with a salt molarity up to 400 mM was investigated. In Example 12 (FIGS. 43A to 44D) a Tris/Acetate buffer with increasing Tris molarity and increasing conductivity, but lacking an antichaotropic salt, was used.

Depending on the hydrophobicity of the mab and the presence of an antichaotropic salt, different RNA reduction values were measured.

For Example 12 (no antichaotropic salt) the investigated mabs show a RNA log reduction range of 4-5 only for a low Tris/Acetate molarity <100 mM (corresponding to a conductivity of <3 mS/cm) independent of the mab hydrophobicity (no significant difference between hydrophilic and hydrophobic mabs). For the hydrophilic mabs 1 and 2 a log reduction value of 4-5 was measured only for salt molarities <25 mM (conductivity <1.5 mS/cm).

In contrast to Example 12, an improved RNA reduction was observed for the hydrophilic mabs in the presence of Na₂SO₄ (Example 11). For hydrophilic mab1 a RNA log reduction range of 4-5 was observed up to a salt molarity of 100 mM Na₂SO₄ at pH 5, corresponding to a conductivity of <9.4 mS/cm. For pH 8 a log reduction value of 4-5 was measured for molarities <40 mM (corresponding to a conductivity of 4 mS/cm). For hydrophilic mab2 a RNA log reduction range of 4-5 was observed up to a salt molarity of 225 mM, corresponding to a conductivity of <19 mS/cm. For the hydrophobic mabs no significant increase in RNA reduction was observed in the presence of an antichaotropic salt.

1. A method for producing an antibody using a mixed mode chromatography material that comprises ion exchange functional groups and hydrophobic interaction functional groups (MM HIC/IEX) operated in flowthrough mode, wherein
a) the antibody is a hydrophilic antibody, and
b) the antibody is applied in a solution comprising the antibody and an antichaotropic salt to the MM HIC/IEX.

2. The method according to claim 1, wherein the method further comprises the following steps:
c) optionally a rinsing solution is applied,
d) the antibody is recovered in the flowthrough of b) or optionally in the flowthrough of b) and c),
and thereby producing the antibody using a MM HIC/IEX operated in flowthrough mode.

3. The method according to claim 1 or claim 2, wherein
the method is for producing an antibody composition with reduced antibody-related high molecular weight (HMW) impurity content and/or with reduced viral impurity content,
the antibody is applied to the MM HIC/IEX in a solution comprising the antibody, at least one HMW impurity and/or at least one viral impurity and the antichaotropic salt,
the antibody composition with reduced HMW impurity content and/or with reduced viral impurity content is recovered from the flowthrough, and
thereby an antibody composition with reduced HMW impurity content and/or with reduced viral impurity content is produced.

4. The method according to claim 3, wherein the HMW impurity content and/or the viral impurity content is reduced compared to the solution applied to the MM HIC/IEX in step b).

5. The method according to claim 3, wherein the HMW impurity content and/or the viral impurity content is reduced compared to a solution essentially without an antichaotropic salt; and/or compared to a solution comprising a hydrophobic antibody.

6. The method according to claim 1, wherein the hydrophilic antibody is an antibody that has a retention time on a hydrophobic interaction chromatography (HIC) material that is equal or less than that of rituximab.

7. The method according to claim 6, wherein the HIC material contains polyether groups (ethyl ether groups) as ligand.

8. The method according to claim 1, wherein the antichaotropic salt has a molar surface tension increment in the range of and including 1.285 to 4.183×10E3 dyn*g*cm⁻¹*mol⁻¹.

9. The method according to claim 1, wherein the antichaotropic salt is selected from the group consisting of (NH₄)₂SO₄, Na₂SO₄, K₂SO₄, NaCl and KCl.

10. The method according to claim 1, wherein the solution comprising the antibody and the antichaotropic salt of step b has a conductivity of from 0.5 to 120 mS/cm.

11. The method according to claim 1, wherein in the solution comprising the antibody and the antichaotropic salt, the antichaotropic salt has a concentration of from 10 mM to 900 mM.

12. The method according to claim 1, wherein the loaded amount to the MM HIC/IEX is from 15 g of protein per Liter of chromatography material (15 g/L) to 350 g of protein per Liter of chromatography material (350 g/L).

13. The method according to claim 1, wherein the solution comprising the antibody and the antichaotropic salt has a pH value of from 4.0 to 9.0.

14. The method according to claim 3, wherein the HMW impurity is an impurity which has a molecular weight of 285 kDa or more.

15. The method according to claim 1, wherein the MM HIC/IEX comprises
i) anion exchange functional groups or cation exchange functional groups, or
ii) strong anion exchange functional groups, or
iii) weak cation exchange functional groups.
